(19) United States
(12) Patent Application Publication
(10) Pub. No.: US 2023/0407293 A1
(43) Pub. Date: Dec. 21, 2023

(54) DNA-ENCODED AND AFFINITY-TAGGED MASKED-WARHEAD COMPOUNDS AND USE THEREOF IN ASSEMBLING LIBRARIES OF SMALL MOLECULES ENABLED FOR LATE-STAGE PURIFICATION AND MULTIPLEXED SCREENING OF COVALENT LIGANDS

(71) Applicant: Juan Pablo Maianti, Winchester, MA (US)

(72) Inventor: Juan Pablo Maianti, Winchester, MA (US)

(21) Appl. No.: 18/126,250

(22) Filed: Mar. 24, 2023

Related U.S. Application Data
(63) Continuation-in-part of application No. PCT/US2022/078783, filed on Oct. 27, 2022.
(60) Provisional application No. 63/272,317, filed on Oct. 27, 2021.
(60) Provisional application No. 63/323,825, filed on Mar. 25, 2022.

(51) Int. Cl. C12N 15/10; C07K 14/00

(52) U.S. Cl. CPC C12N 15/1065; C12N 2320/11; C07K 14/001

(57) ABSTRACT

The present disclosure relates to DNA-encoded and affinity-tagged masked warhead installation compounds and use thereof in the synthesis of electrophilic warhead DNA-Encoded Libraries (eDEL), including substituted and unsubstituted acrylamide warheads, which can be purified from unreacted library intermediates and byproducts.

### RELATED APPLICATIONS

This application claims the benefit of priority under 35 U.S.C. § 119(e) to U.S. Provisional Application No. 63/272,317, filed Oct. 27, 2021, U.S. Provisional Application No. 63/323,825, filed Mar. 25, 2022, and PCT Application No. PCT/2022/78783, filed on Oct. 27, 2022, each of which is incorporated herein by reference in its entirety.

### BACKGROUND

Covalent mechanisms have the potential to massively expand the fraction of the proteome targeted by small-molecules, and by consequence expand the scope of biomedical problems that can be intervened. However, only a small fraction of all known and unknown pockets having reactive residues has ever been explored with covalent ligands. Across academia and the pharmaceutical sector, there is a scarcity of structurally diverse screening collections having appropriately validated warheads, and a lack of technologies to identify covalently targetable pockets in allosteric and non-catalytic domains.

The desirable balance of k_{inact}/Kᵢ parameters of highly selective covalent inhibitors that make them valuable commodities also obfuscates their discovery using conventional screening infrastructure and small-molecule collections devised for reversible ligands including DNA-encoded libraries (DEL), High-Throughput Screening (HTS), and Fragment-Based Screening (FBS) (Goodnow et al., Nat. Rev. Drug Discov. 16(2):131-147 (2017); Volochnyuk et al., Drug Discov. Today 24(2):390-402 (2019); Lu et al., RSC Chem. Biol. 2(2):354-367 (2021); Parker et al., Cell 168(3):27-41.e29 (2017)). Complementary methods such as activity-based proteomics can rank-order the reactivity of protein residues using warhead scouting ligands, but pose significant hurdles for screening collections larger than a few thousand compounds (Hacker et al., Nat. Chem. 9(12):1181-1190 (2017); Kuljanin et al., Nat. Biotechnol. 39(5):630-641 (2021)). Implementing warhead reagents (e.g., reactive olefins, acryloyl halides or equivalent synthons) on established DEL collections has been hindered by two levels of incompatibility: first, at the level of the combinatorial chemistry routes optimized for the presence of DNA and water; and secondly, at the level of the in vitro selection workflows (e.g., one-round enrichment and background noise) (Guilinger et al., Bioorg. Med. Chem. 42:116223 (2021); Zimmermann et al., Chem.-Eur. J. 23(34): 8152-8155 (2017); Zambaldo et al., MedChemComm 7(7):1340-1351 (2016); Kuai et al., SLAS Discov. Adv. Sci. Drug Discov. 23(5):405-416 (2018); Zhu et al., SLAS Discov. Adv. Sci. Drug Discov. 24(2):169-174 (2019); Cochrane et al., ACS Comb. Sci. 21(5):425-435 (2019)). An additional hurdle previously observed when implementing warheads is that conventional DEL preparations generate inseparable mixtures (Clark et al., Nat. Chem. Biol. 5(9):647-654 (2009); Shi et al., RSC Adv. 11(4):2359-2376 (2021)). Lack of purity can confound hit identification and screening outcomes because identical DNA barcodes are connected to unreacted intermediates and byproducts (Zambaldo et al., MedChemComm 7(7):1340-1351 (2016); Kuai et al., SLAS Discov. Adv. Sci. Drug Discov. 23(5):405-416 (2018); Zhu et al., SLAS Discov. Adv. Sci. Drug Discov. 24(2):169-174 (2019)).

### SUMMARY

A first aspect of the present disclosure is directed to compounds represented by formula (I) or (II), or a pharmaceutical salt or stereoisomer thereof:

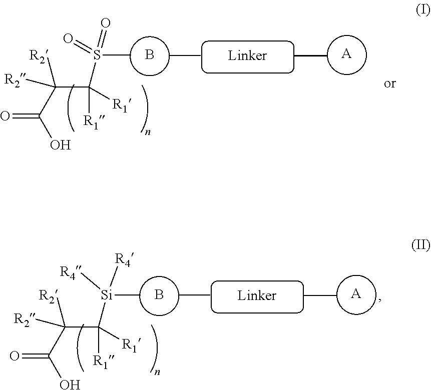

wherein [symbol]
, [symbol] , [symbol] , R₁′, R₁″, R₂′, R₂″, R₄′, R₄″ and n are defined herein.

In another aspect of the present disclosure, methods of making the compounds are provided.

Another aspect of the present disclosure is directed to methods of creating an electrophilic warhead-bearing DNA-Encoded Library (eDEL) comprising:
coupling the compound of formula I or II with a DNA-Encoded Library (DEL) to generate a stable masked-warhead bearing DEL intermediate library (mwDEL); purifying the mwDEL intermediate library from unreacted and byproduct entities; unmasking the mwDEL intermediate to generate a warhead-bearing eDEL; and
purifying the warhead-bearing eDEL.

In some embodiments, the stable masked-warhead bearing mwDEL intermediate comprises an arylsulfone comprising [symbol] .

Another aspect of the present disclosure is directed to an eDEL, which is generated from the methods described herein.

In some embodiments, the eDEL is used in an in vitro selection assay followed by DNA sequencing. In some embodiments, the in vitro selection assay is used for screening protein ligands. In some embodiments, the eDEL is used for screening ligands that covalently modify a residue of a protein (e.g., the thiol group of Cysteine, the amino group of Lysine, the imidazole group of Histidine, the hydroxyl groups of Serine, Threonine or Tyrosine, etc.).

Disclosed are masked-warhead installation reagents that, upon unmasking afford acrylamide-class warheads attached to DEL (FIG. 1A-FIG. 1), which altogether solve the myriad incompatibilities that have hindered the assembly, purification and screening of warhead-bearing DELs and covalent ligands in the past (FIG. 2A). The masked-warhead reagents can be coupled directly on DELs featuring traditional nucleophilic functional groups (e.g. primary or secondary amines, anilines, thiols, hydroxyls, phenols, etc.). The present disclosure can be implemented to solve the scarcity of warhead-bearing small molecule collections, resulting in structurally diverse libraries amenable for covalent ligand screening comprising both therapeutically validated and unexplored warhead classes (Goodnow et al., Nat. Rev. Drug Discov. 16(2):131-147 (2017)).

The presently described methodologies and resultant compositions/products are useful for solving the synthetic and the purification challenges associated with the incompatible circumstances of implementing reactive warheads (e.g., acrylamides) in the context of DNA-encoded combinatorial chemistry (Shi et al., RSC Adv. 11(4):2359-2376 (2021)), which is a process requiring compatibility with split-and-pooled mixed library formats, in the presence of water and DNA, all the while promoting efficient conversion of warhead attachment onto the vastly different nucleophilic groups typically encountered in a DEL library (e.g., anilines, sterically hindered amines, hydroxyls, heterocyclic nitrogens, etc.) (Clark et al., Nat. Chem. Biol. 5(9):647-654 (2009)). The masked-warhead reagents and methods of the present disclosure are useful for installing the described acrylamide-type warheads on a panoply of small-molecule classes that can be generated by combinatorial chemistry with DNA barcodes (Shi et al., RSC Adv. 11(4):2359-2376 (2021)).

### BRIEF DESCRIPTION OF THE DRAWINGS

FIG. 1A illustrates the preliminary art of highly desirable acrylamide-class warheads that are challenging to implement on DNA-Encoded Libraries of small molecules (1A-1 to -7).

FIG. 1B illustrates the thought process behind the disclosure of the warhead-masking reagents (represented by structures 1B-1 to -3), compared to other reactions that are deemed problematic and/or incompatible to implement warheads on DNA-Encoded Libraries of small molecules. Advantages of sulfonylaryl warhead-masking approach (1B-1): 1) Cα and Cβ sulfonylaryl masking options; 2) versatile assembly of sulfonylaryl reagents; 3) broad coverage of useful acrylamide substitutions; 4) compatible with water, DNA, and affinity tags (e.g., biotin); 5) tunable reactivity (e.g., R₃═OMe, vs. R₃═H, vs. R₃═F in structure 1B-1); 6) scalable synthesis (e.g., from 3-mercapto-4-nitrobenzoic acid). Disadvantages of phosphonate warhead-masking approach: 1) synthetic limitations to Cα masking as shown in structure 1B-2; 3) few useful options for acrylamide warhead substitution; 3) stabilized enolates may expell aldehyde instead of proceeding to olefination. Disadvantages of alkylsilyl warhead-masking approach: 1) synthetic limitations to stable CP masking as shown in structure 1B-3; 2) difficult to isolate a-hydroxy-p-silyls due to fast elimination; 3) few useful options for acrylamide warhead substitution; 4) challenging to produce; and 5) limited reactivity tuning. For similar reasons, other named reactions we have deemed unsuitable for masking warheads in eDEL include: Bamford-Stevens, McMurry, Shapiro, Tebbe, Petasis, Takai, Kauffmann, Wharton, Seigrist, Barton-Kellogg, Wittig, Rupe, Chugaey, Ramberg-Backlund, Stobbe, Perkin, Knoevenagel, Burges, Garegg-Samuelson, Boord, Corey-Winter, Hoffmann, and Grieco.

FIG. 1C is an illustrative example of masked warhead reagents (represented by structure 1C-1) implemented on a generic secondary-amine DNA-encoded library (DEL) synthesized by 3 rounds of split-and-pool combinatorial synthesis, comprising the desired intermediate represented by structure 1C-2 (wherein building blocks are depicted as circles labeled A, B, and C) and also the byproducts of the DEL synthesis (represented by structure 1C-3). When the reagent 1C-1 comprises a DNA sequence complementary to part of the DNA barcode of the DEL it can undergo base-pairing (1C-Rn1) to give 1C-4, followed by standard amide coupling or DNA-templated amide bond formation (1C-Rn2). This amide coupling generates a stable masked-warhead DEL intermediate (mwDEL) represented by structure 1C-6. The mwDEL 1C-6 can be purified in step 1C-Rn3 to pure 1C-7 using gel electrophoresis (or an affinity tag), which removes the accompanying capped-, unreacted-, and byproduct-DEL entities (represented by structure 1C-5, depicted as X and missing building-block C of the DEL). Finally, an unmasking reaction in step 1C-Rn4 (e.g., with base or alkaline buffer) liberates sulfur dioxide and removes the remainder of the warhead-masking reagent to afford the purified activated warhead-bearing eDEL represented by structure 1C-8.

FIG. 2A shows the challenges associated with the implementation of acrylamide-class warheads (represented by structure 2A-2) on DNA-encoded libraries in the presence of DNA and water. Prior art has shown the following issues: 1) the 2A-1 reagents are unstable starting materials; 2) the 2A-1 reagents are incompatible with DNA and water; 2) the 2A-1 reagents provide limited options of R1 and R2; 3) the 2A-1 reagents have low reactivity with hindered amines and anilines; 4) the product 2A-2 comprises a crude mixture with DNA barcodes that cannot be separated; 5) the product 2A-2 comprises an olefin that is incompatible with further chemistry; 6) the eDEL crude product 2A-2 has limited shelf-life and storage options.

FIG. 2B shows an example of the claimed masked warhead reagents (2B-1) offering versatility for the introduction of steric and reactivity-tuning substituents on the arylsulfone intermediate and the the resulting activated warhead-bearing eDEL (2B-2) and the benzothiazolone removed during purification (2B-3).

FIG. 2C shows alternative examples of masked warhead reagents (2C-1, -4, -6, -8) offering versatility for the use of many classes of arylsulfone moieties, including benzothiazole sulfones (2C-1), pyridine sulfones (2C-4), pyrimidine sulfones(2C-4), alkyl-tetrazole sulfones (2C-6), and phenyl-tetrazole sulfones(2C-8), to afford activated warhead-bearing eDELs (2C-2) and the respective intermediates removed during purification (2C-3).

FIG. 3 ¹H-NMR spectrum of a benzothiazole sulfone masked-warhead reagent.

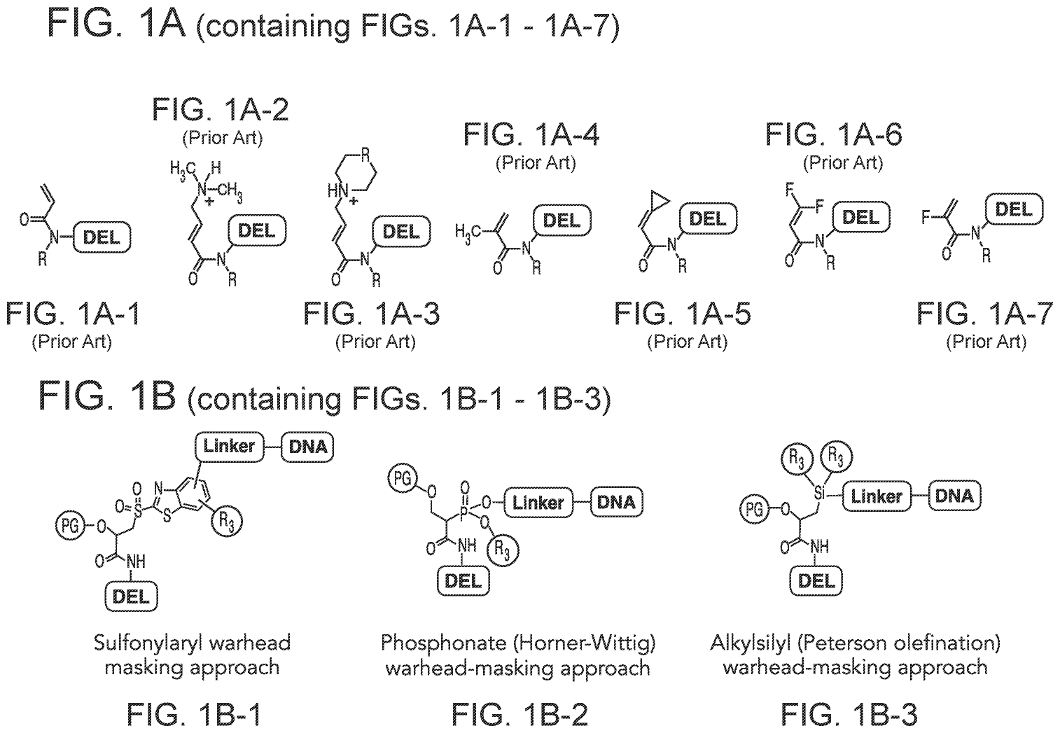

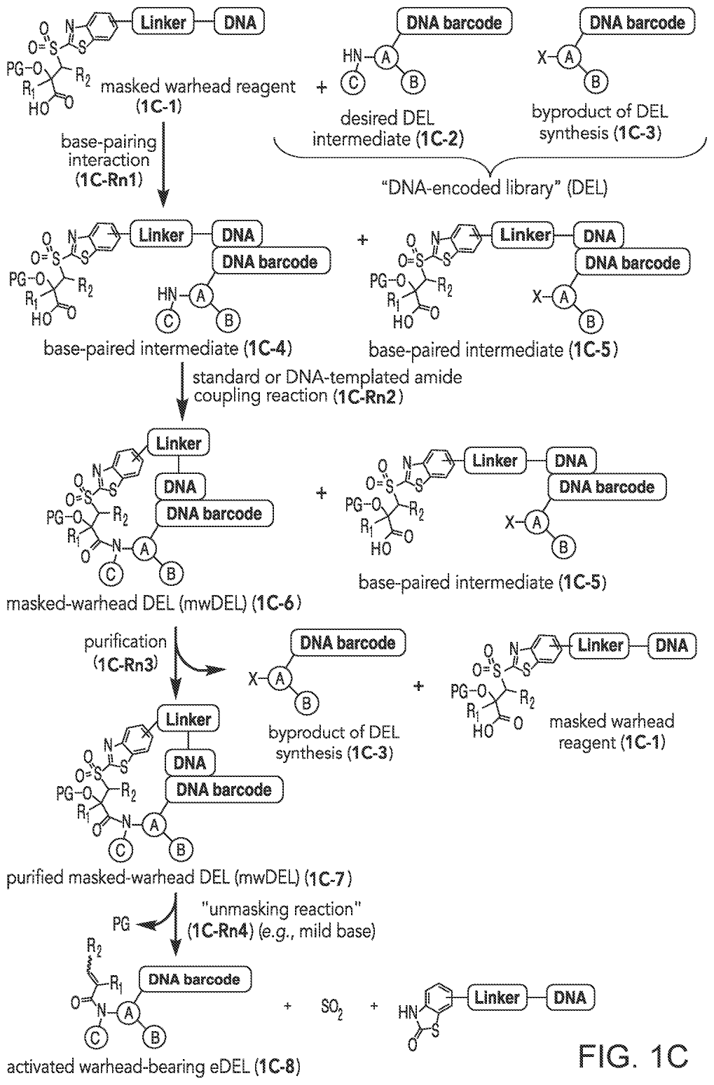

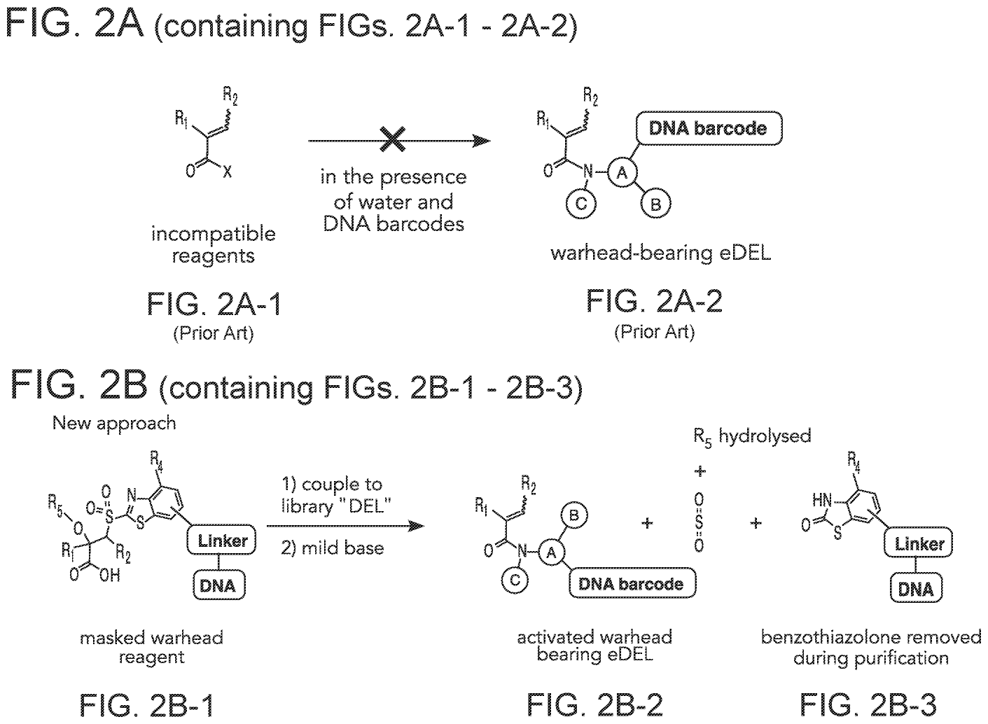

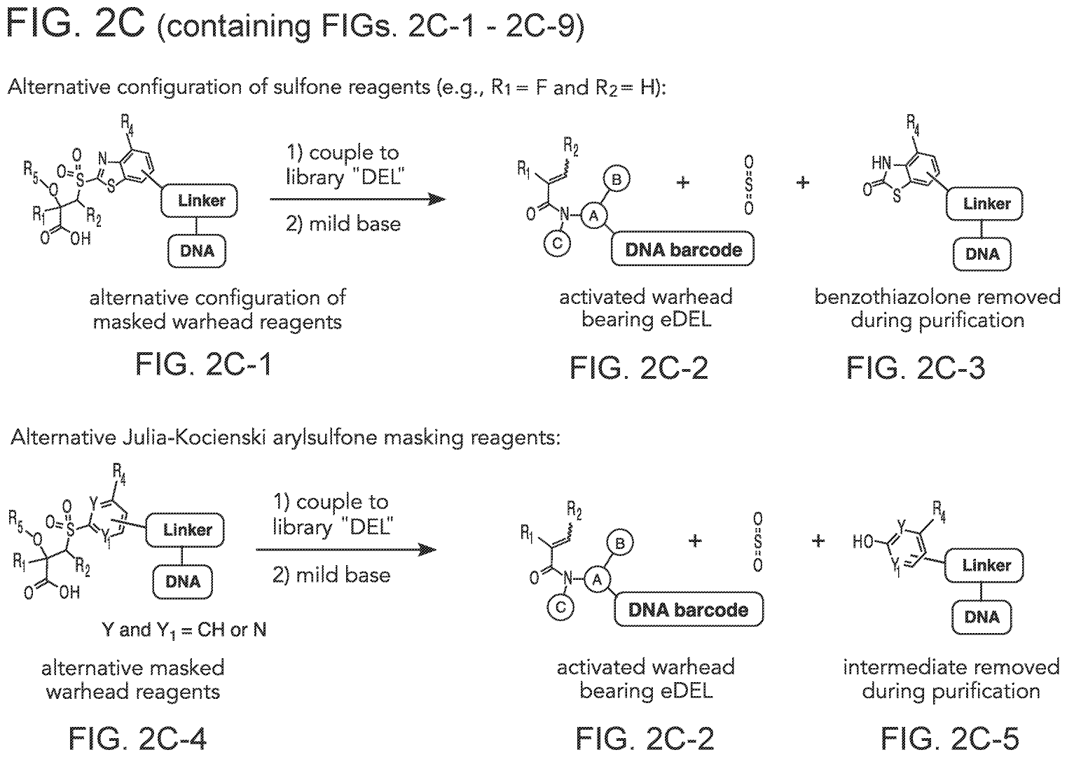

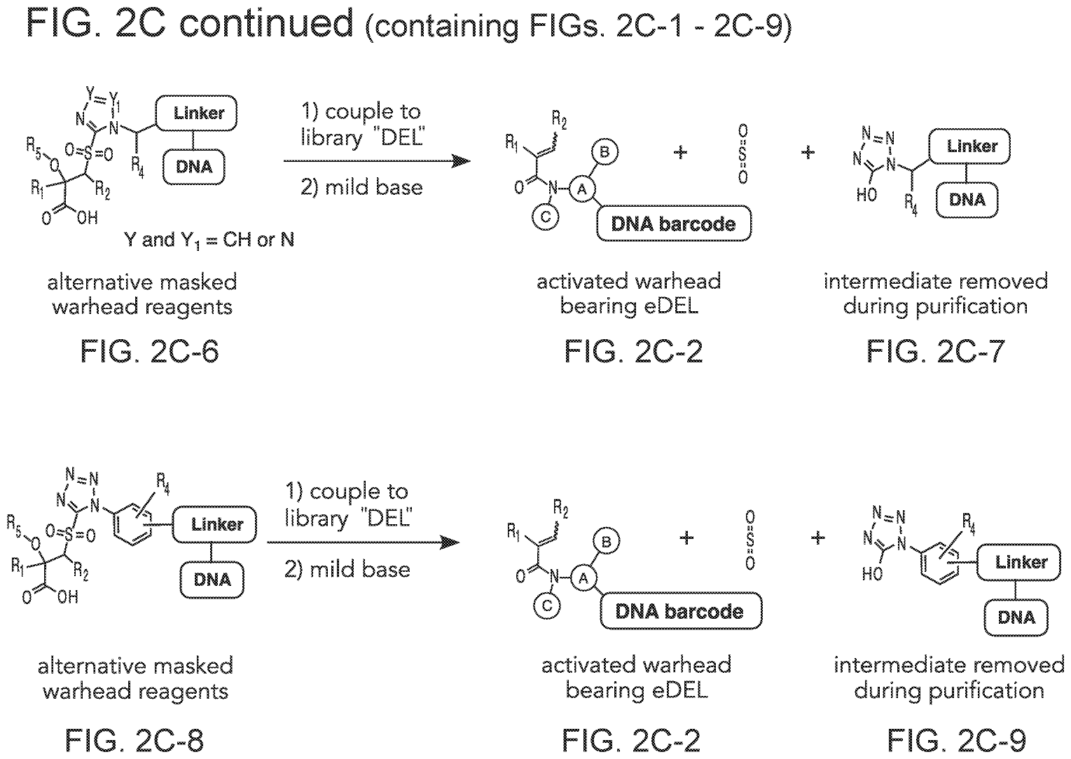

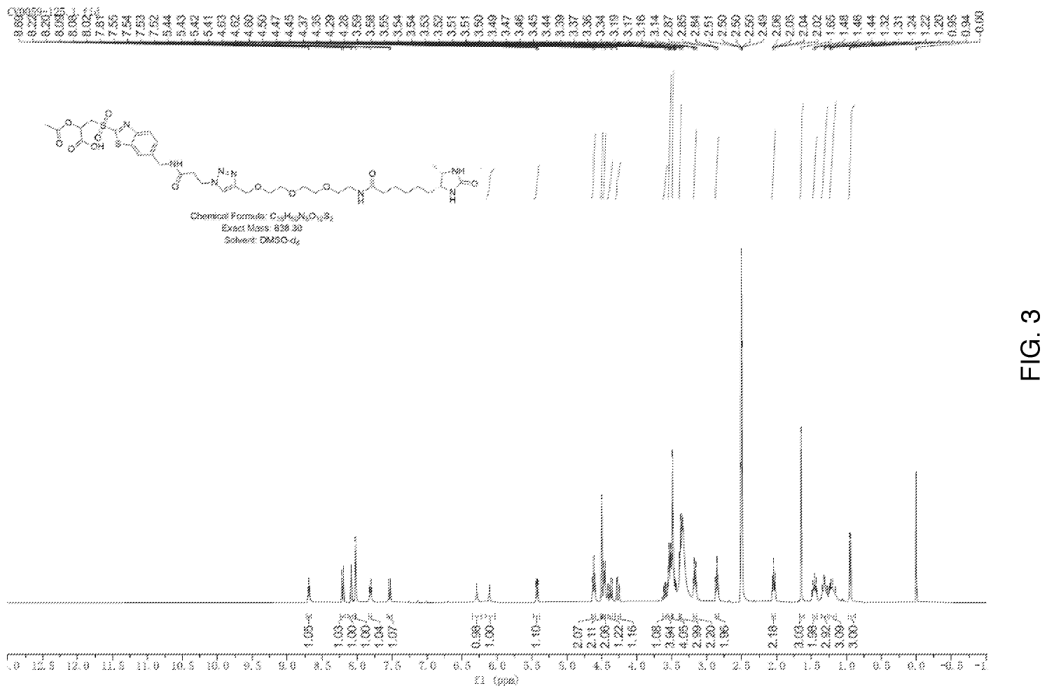

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like. Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to the disclosed compounds, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₅, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₅ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “alkoxylene” refers to a saturated monovalent aliphatic radicals of the general formula (—O—CₘH₂ₘ—) where m represents an integer (e.g., 1, 2, 3, 4, 5, 6, or 7) and is inclusive of both straight-chain and branched-chain radicals. The alkoxylene chain may be attached to the rest of the molecule through a single bond and to the radical group through a single bond. In some embodiments, the alkoxylene group contains one to 3 carbon atoms (—O—C₁-C₃ alkoxylene). In other embodiments, an alkoxylene group contains one to 5 carbon atoms (—O—C₁-C₅ alkoxylene).

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocyclyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{C} is an alkylene chain. The term carbocyclic also embraces carbocyclylalkoxy groups which as used herein refer to a group bonded through an oxygen atom of the formula —O—R^{c}-carbocyclyl where R^{C} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring. The structure of any aryl group that is capable of having double bonds positioned differently is considered so as to embrace any and all such resonance structures.

Thus, the term aryl embraces aralkyl groups (e.g., benzyl) which as disclosed above refer to a group of the formula —R^{c}-aryl where R^{C} is an alkylene chain such as methylene or ethylene. In some embodiments, the aralkyl group is an optionally substituted benzyl group.

The term aryl also embraces aralkoxy groups which as used herein refer to a group bonded through an oxygen atom of the formula —O—R^{c}-aryl where R^{C} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₅ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur and oxygen. In some embodiments, heterocyclyl includes 4- to 6-membered monocycles having one or more heteroatoms selected from nitrogen, sulfur and oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring. The structure of any heteroaryl group that is capable of having double bonds positioned differently is considered to embrace any and all such resonance structures.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may thus include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The substituent present on an oxygen atom is an oxygen protecting group (also referred to herein as an “hydroxyl protecting group” or “—PG”). Oxygen protecting groups include, but are not limited to, —R^{aa}, —N(R^{bb})₂, —C(═O)SR^{aa}, —C(═O)R^{aa}, —CO₂R′, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{cc})₂, —P(═O)(OR^{cc})₂, and —P(═O)(N(R^{bb})₂)₂, wherein X⁻, R^{aa}R^{bb}, and R^{cc}, wherein:

each instance of R^{aa} is, independently, selected from C₁₋₁₀ alkyl, C₁₋₁₀ haloalkyl, C₁₋₁₀perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂—CN, —C(═O)R^{aa}, μC(═O)N(R^{cc})₂, —CO₂R^{aa}, —SO₂R^{aa}, —C(═NR^{cc})OR^{aa}, —C(═NRC)N(R^{cc})₂, —SO₂N(R^{cc})₂, —SO₂R^{cc}, —SO₂OR^{cc}, —SOR^{cc}, —C(═S)N(R^{cc})₂, —C(═O)SR^{cc}, —C(═S)SR^{cc}, —P(═O)(R^{aa}, —P(═O)(OR^{cc}), —P(═O)(N(R^{cc})₂)₂, C₁₋₁₀ alkyl, C₁₋₁₀ haloalkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X is a counterion;

each instance of R^{cc} is, independently, selected from hydrogen, C₁₋₁₀ alkyl, C₁₋₁₀ haloalkyl, C₁₋₁₀perhaloalkyl, C₂₋₁₀ alkenyl, C₂₋₁₀ alkynyl, heteroC₁₋₁₀ alkyl, heteroC₂₋₁₀ alkenyl, heteroC₂-10 alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(Re)₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(Re)₂, —OC(═O)N(Re)₂, —NR^{ff}C(═O)R^{ee}, —NR^{ff}CO₂R^{ee}, —NR^{ee}C(═O)N(Re)₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ee})N(Re)₂, —NR^{ff}C(═NR^{ee})N(Re)₂, —NR^{ff}SO₂R^{ee}, —SO₂N(Re)₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ haloalkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ haloalkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ haloalkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “Click chemistry” has been applied to a collection of reliable and self-directed organic reactions (Kolb et al., Angew. Chem. Int. Ed., 40:2004-2021 (2001); Fantoni et al., Chem. Rev. 121(12):7122-7154 (2021)).

The term “binding” as it relates to interaction between targeted protein/s and a member of the eDEL collection, including the warhead, typically referring to an inter-molecular or a covalent interaction that is preferential (also referred to herein as “selective”) in that binding of the member of the eDEL collection, including the warhead, with other proteins present in the cell is substantially less and, in some cases, may be functionally insignificant.

Broadly, the compounds of the present disclosure are represented by formula (I) or a pharmaceutical salt or stereoisomer thereof:

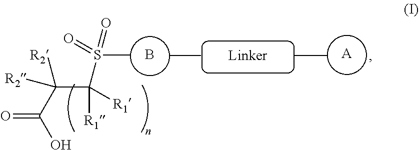

wherein

[symbol] is an oligonucleotide tag represented by [symbol] or an affinity tag;

[symbol] is a linker that covalently attaches [symbol] to [symbol] ;

[symbol] is

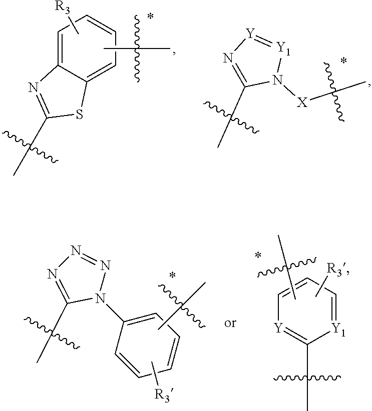

wherein

[symbol] is the connection to the sulfone group,

[symbol] is the connection to the linker,

R₃′ is H, halogen, amino, hydroxyl, (C₁-C₆) alkyl, (C₁-C₆) hydroxyalkyl, (C₁-C₆) aminoalkyl, (C₃-C₆) carbocyclyl, 4- to 6-membered heterocyclyl, (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different Ria groups, wherein each Ria is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl,

X is (C₁-C₆) alkyl, wherein said alkyl is further optionally substituted by one or more, identical or different Ria groups, wherein each Ria is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl, and

Y and Y₁ are each independently CH or N;

R₁′ and R₁″ are each independently H, CH₃, CF₃, halogen, CH₂NMe₂,

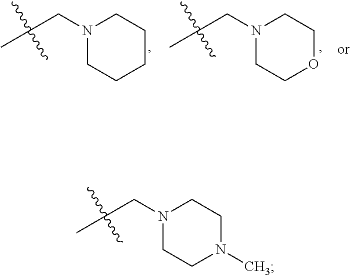 [SMILES: *CN1CCCCC1 ; *CN1CCOCC1 ; *CN1CCN(C)CC1]

R₂′ and R₂″ are each independently H, halogen, CF₃, OH, OAc, CH₂OH, CH(CH₃)OH, C(CH₃)₂OH, CH₂OAc, CH₂OPG,

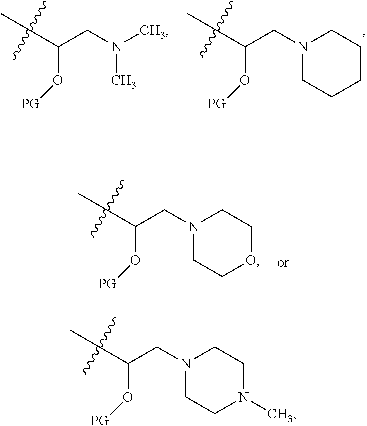

wherein PG is a protecting group, provided that both R₂′ and R₂″ are not H, or R₂′ and R₂″ can be joined to form ═O; and
n is 0 or 1.

In some embodiments, the compounds of the present disclosure are represented by formula (II) or a pharmaceutical salt or stereoisomer thereof:

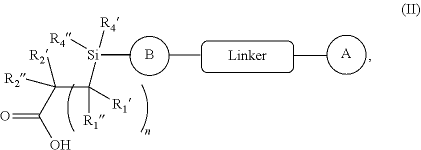

wherein

[symbol] is an oligonucleotide tag represented by [symbol] or an affinity tag;

[symbol] is a linker that covalently attaches [symbol] to [symbol] ;

[symbol] is

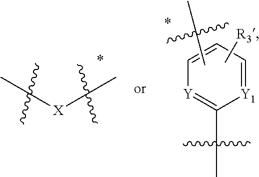

wherein

[symbol] is the connection to the silyl group,

[symbol] is the connection to the linker,

R₃′ is H, halogen, amino, hydroxyl, (C₁-C₆) alkyl, (C₁-C₆) hydroxyalkyl, (C₁-C₆) aminoalkyl, (C₃-C₆) carbocyclyl, 4- to 6-membered heterocyclyl, (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different Ria groups, wherein each Ria is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl,

X is (C₁-C₆) alkyl, wherein said alkyl is further optionally substituted by one or more, identical or different Ria groups, wherein each Ria is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl, and

Y and Y₁ are each independently CH or N;

R₁′ and R₁″ are each independently H, CH₃, halogen, CH₂NMe₂,

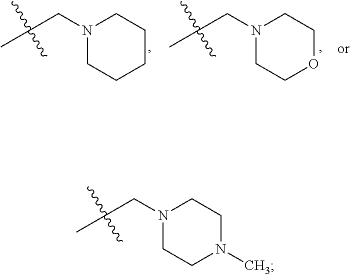 [SMILES: *CN1CCCCC1 ; *CN1CCOCC1 ; *CN1CCN(C)CC1]

R₂′ and R₂″ are each independently H, CF₃, halogen, OH, OAc, CH₂OH, CH(CH₃)OH, C(CH₃)₂OH, CH₂OAc, CH₂OPG,

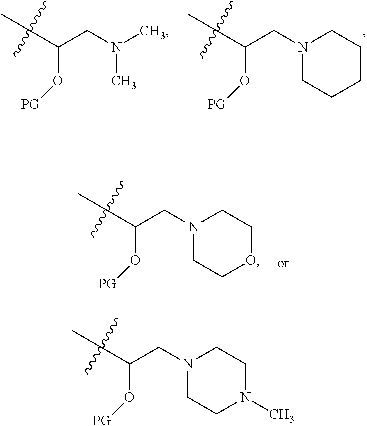

wherein PG is a Protecting Group, provided that both R₂′ and R₂″ are not H, or R₂′ and R₂″ can be joined to form ═O;

R₄′ and R₄″ are each independently alkyl or aryl; and

n is 0 or 1.

In some embodiments, R₄′ and R₄″ are each independently CH₃, CH₂CH₃, CF₃, propyl, isopropyl, butyl, isobutyl, alkyl, or phenyl.

In some embodiments, [symbol] is [symbol] .

In some embodiments, n is 0.

In some embodiments, R₂′ and R₂″ are each independently H, CF₃, halogen, CH₂OAc, CH₂OH, CH(CH₃)OH, C(CH₃)₂OH, CH₂OPG,

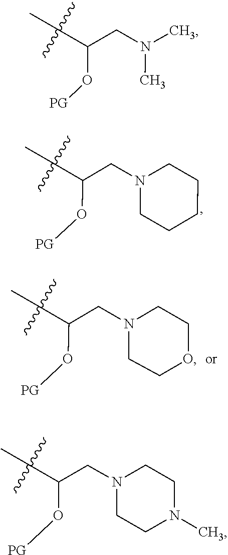

or R₂′ and R₂″ can be joined to form ═O, and PG is a protecting group. In some embodiments, PG is an acyl group (e.g., acetyl, benzoyl, chloroacetyl, dichloroacetyl, trichloroacetyl, trifluoroacetyl, methoxyacetyl, o-(dibromomethyl)benzoyl, etc.). In some embodiments, PG is a carbonate group (e.g., methyl carbonate, 9-fluorenylmethyl carbonate, trichloroethyl carbonate (Troc), 2-(trimethylsilyl)ethyl carbonate (TMSEC), isobutyl carbonate, methyl dithiocarbonate, p-methoxybenzyl carbonate, etc.). In some embodiments, PG is a carbamate group (e.g., methylamine carbamate, alkyl N-phenylcarbamate, etc.). In some embodiments, PG is an ether group (e.g., methoxylmethyl (MOM), benzyl, p-methoxybenzyl, methylthiomethyl ether (MTM), tetrahydropyranyl (THP), etc.). In some embodiments, PG is a silyl group (e.g., trimethylsilyl (TMS), triethylsilyl (TES), diethylisopropylsilyl (DEIPS), t-butyldimethylsilyl (TBDMS), diphenylmethylsilyl (DPMS), t-butyldimethylsilyl (TBS), etc.).

In some embodiments, R₂′ and R₂″ are each independently H or F.

In some embodiments, [symbol] is

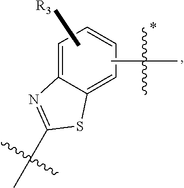

and the compound of formula I is represented by structure:

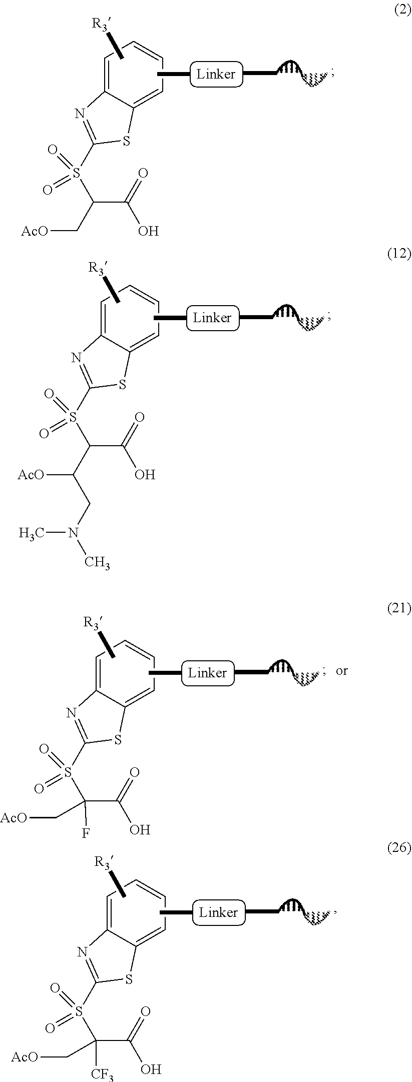

or a pharmaceutical salt or stereoisomer thereof.

In some embodiments, [symbol] is

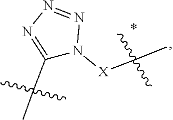

and the compound of formula I is represented by structure:

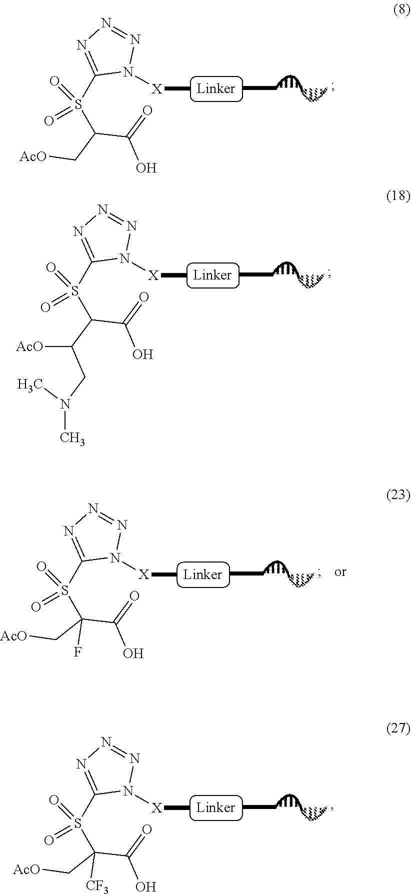

or a pharmaceutical salt or stereoisomer thereof.

In some embodiments, [symbol] is

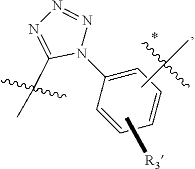

and the compound of formula I is represented by structure:

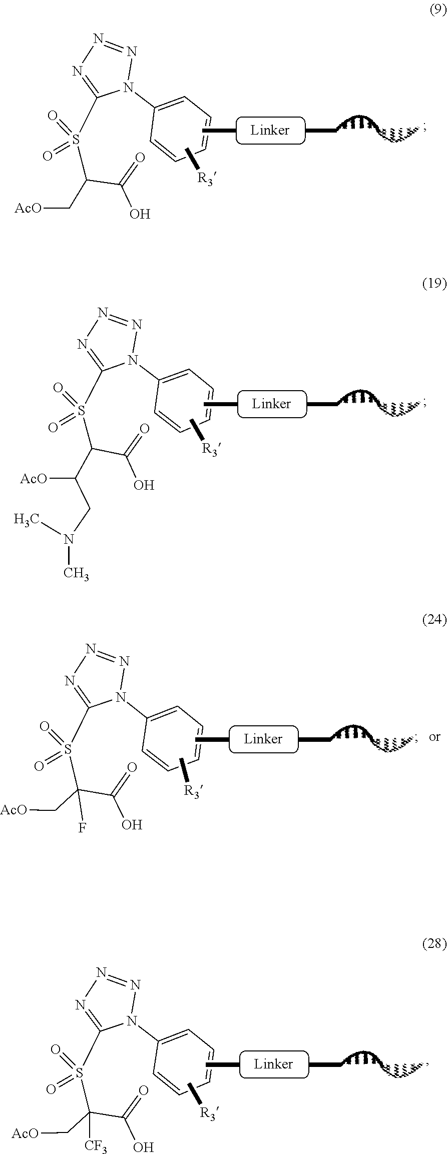

or a pharmaceutical salt or stereoisomer thereof.

In some embodiments, [symbol] is

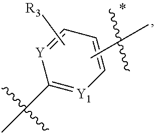

and the compound of formula I is represented by structure:

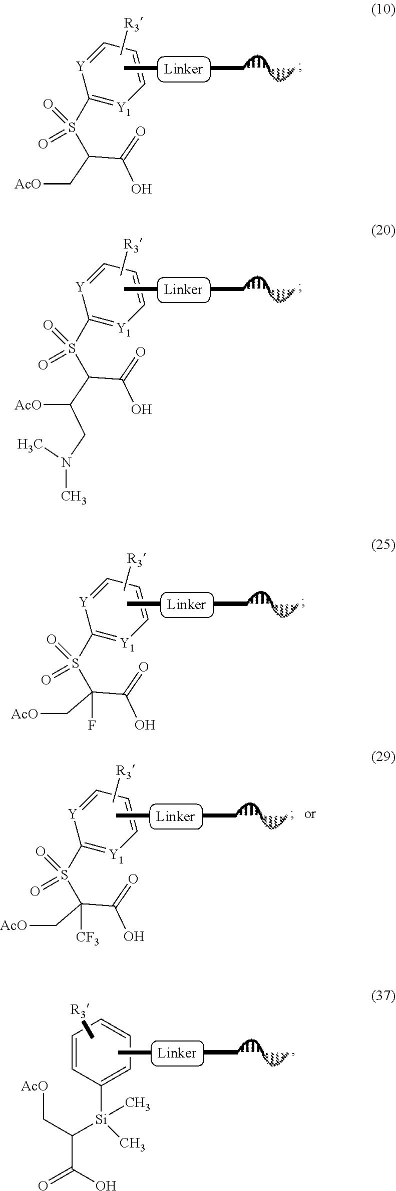

or a pharmaceutical salt or stereoisomer thereof.

In some embodiments, n is 1.

In some embodiments, R₁′ and R₁″ are each independently H or CH₂NMe₂.

In some embodiments, R₁′ and R₁″ are each independently H or

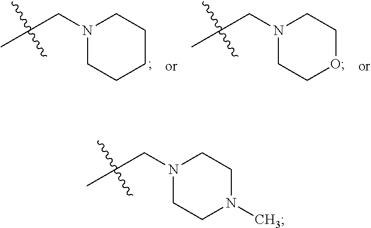 [SMILES: *CN1CCCCC1 ; *CN1CCOCC1 ; *CN1CCN(C)CC1]

In some embodiments, R₁′ and R₁″ are both H.

In some embodiments, R₂ and R₂″ are each independently H or acetate.

In some embodiments, [symbol] is

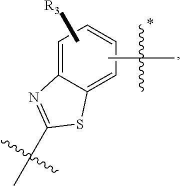

and the compound of formula I is represented by structure:

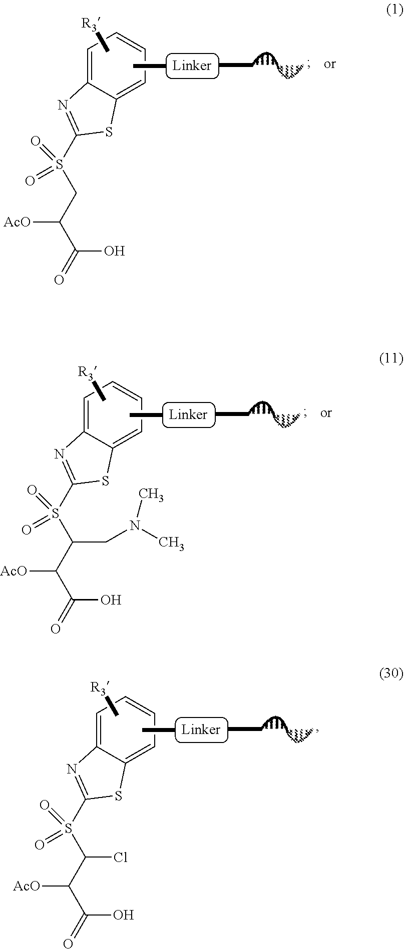

or a pharmaceutical salt or stereoisomer thereof.

In some embodiments, [symbol] is

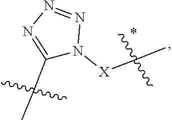

and the compound of formula I is represented by structure:

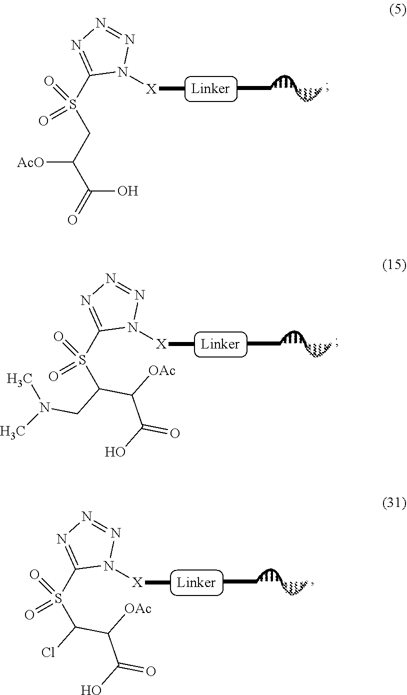

or a pharmaceutical salt or stereoisomer thereof.

In some embodiments, [symbol] is

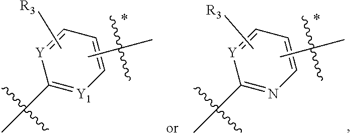

and the compound of formula I is represented by structure:

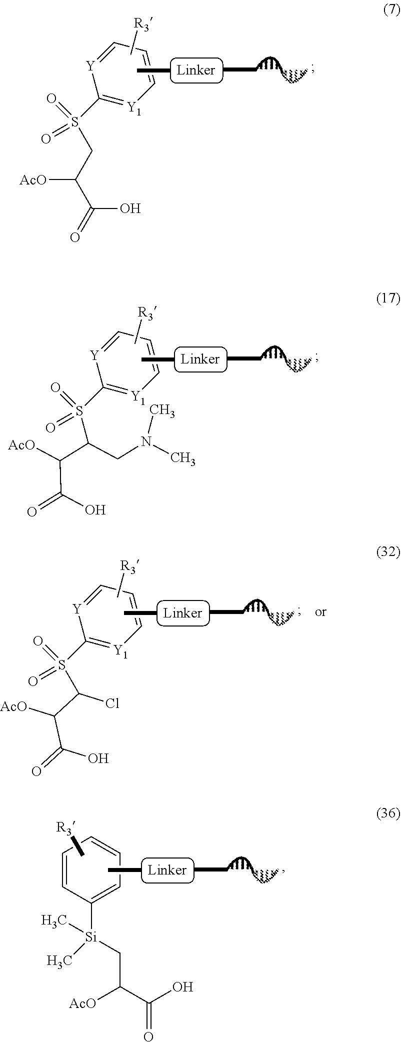

or a pharmaceutical salt or stereoisomer thereof.

In some embodiments, [symbol] is

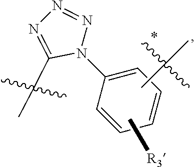

and the compound of formula I is represented by structure:

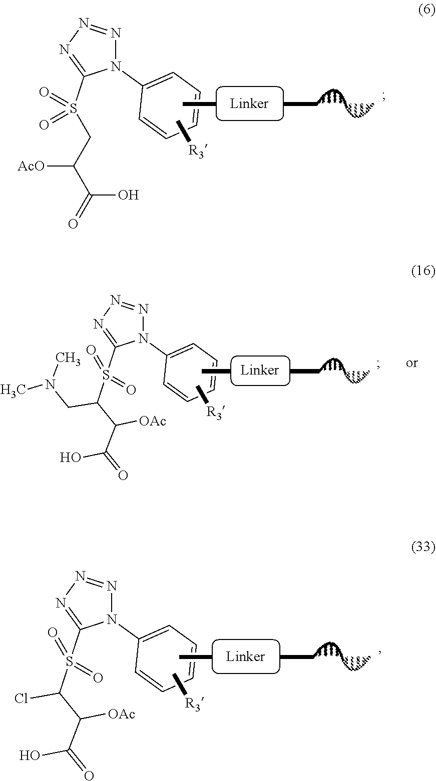

or a pharmaceutical salt or stereoisomer thereof.

In some embodiments, [symbol] is an oligonucleotide tag (e.g., deoxyribonucleic acid (DNA) or ribonucleic acid (RNA) tag) generated by phosphoramidite chemistry (Roy & Caruthers, Molecules. 18(11):14268-14284 (2013)), by polymerase enzymes, by enzymatic ligation of oligonucleotides, or by chemical ligation.

In some embodiments, [symbol] is an oligonucleotide tag comprising natural or unnatural nucleobases; or oligonucleotides of unnatural backbone structures (e.g., phosphodiester backbone, peptide nucleic acid (PNA) backbone, triazole, phosphorothioate ester backbone, and sugar components including ribose, 2-deoxyribose, threose, glycol, fluoro-arabino, 1,5-anhydrohexitol, etc.) (Ochoa & Milam, Molecules. 25(20):4659 (2020)).

In some embodiments, the oligonucleotide tag is a 5′-O-modified DNA bound to the linker L.

In some embodiments, the oligonucleotide tag is a 3′-O-modified DNA bound to the linker L.

In some embodiments, the oligonucleotide tag is a DNA with a modified nucleobase bound to the linker L.

In some embodiments, the oligonucleotide tag is single-stranded.

In some embodiments, the oligonucleotide tag is double-stranded.

In some embodiments, the oligonucleotide tag has a length between 4 and 10 nucleotides.

In some embodiments, the oligonucleotide tag has a length between 10 and 20 nucleotides.

In some embodiments, the oligonucleotide tag has a length between 20 and 50 nucleotides.

In some embodiments, the oligonucleotide tag has a length between 50 and 100 nucleotides.

In some embodiments, the oligonucleotide tag has a length between 100 and 200 nucleotides.

In some embodiments, the oligonucleotide tag has a length between 200 and 500 nucleotides.

In some embodiments, the oligonucleotide tag has base-pairing complementarity to a DEL barcode as described in Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654, which is incorporated by reference.

In some embodiments, [symbol] is an affinity tag. Exemplary affinity tags that may be used in the disclosed compounds are described in Kimple et al., Curr Protoc Protein Sci. 73:9.9.1-9.9.23 (2013) and Lotze et al., Mol Biosyst. 12(6):1731-45 (2016).

In some embodiments, n is 0.

In some embodiments, wherein n is 0, R₂′ and R₂″ are each independently H, halogen, fluorine, CF₃, CH₃, CH₂OAc, CH₂OH, CH(CH₃)OH, C(CH₃)₂OH, CH₂OPG,

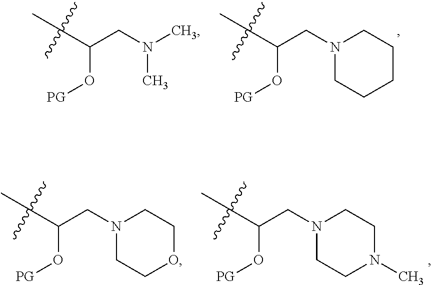

or R₂′ and R₂″ can be joined to form ═O. In some embodiments, the halogen is F.

In some embodiments, [symbol] is

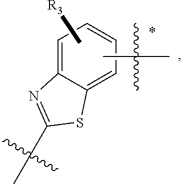

[symbol]

is an affinity tag, and the compound of formula I is represented by structures:

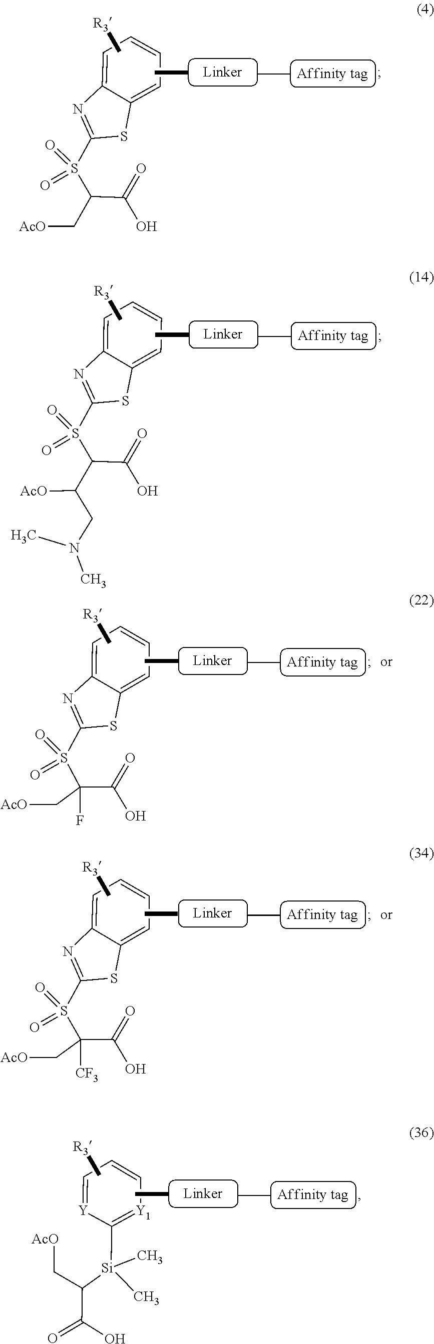

or a pharmaceutical salt or stereoisomer thereof.

In some embodiments, n is 1.

In some embodiments, R₁′ and R₁″ are each independently H or CH₂NMe₂.

In some embodiments, R₁′ and R₁″ are each independently H or

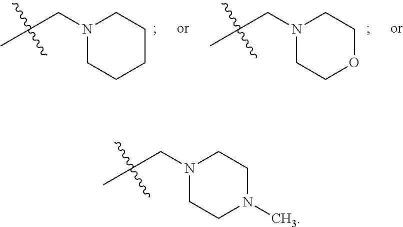 [SMILES: *CN1CCCCC1 ; *CN1CCOCC1 ; *CN1CCN(C)CC1]

In some embodiments, R₁′ and R₁″ are both H.

In some embodiments, [symbol] is

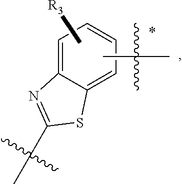

[symbol]

is an affinity tag, and the compound of formula I is represented by structures:

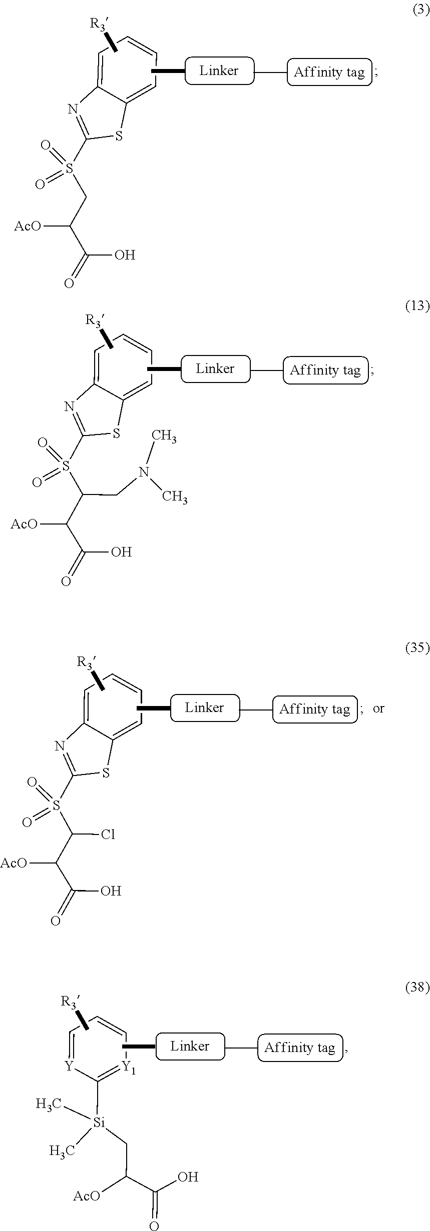

or a pharmaceutically acceptable salt or stereoisomer thereof.

In some embodiments, the affinity tag is biotin or a biotin analog.

In some embodiments, the affinity tag is a peptide epitope for antibody-based affinity purification (e.g., Human influenza hemagglutinin (HA) tag, Myc tag, Flag tag, etc.).

In some embodiments, the affinity tag is a chloroalkyl group (also known as a Halo-tag).

In some embodiments, the affinity tag is a benzylated nucleobase analog (e.g., a SNAP-Tag® or CLIP-Tag™)

### Linkers

The linker (L) provides a covalent attachment between [symbol] and [symbol] .

In some embodiments, the linker (L) is a branched or unbranched, linear or cyclic, substituted or unsubstituted, saturated or unsaturated, group having between 2 and 80 carbon atoms, and optionally having one or more heteroatoms selected from O, N, or S. A variety of linkers are known to one of skill in the art and may be used in the masked warhead compounds described herein. For example, in certain embodiments, L comprises one or more optionally substituted groups selected from amino acids, polyether chains, aliphatic groups, and any combinations thereof. In certain embodiments, L consists of one or more optionally substituted groups selected from amino acids, polyether chains, aliphatic groups, and any combinations thereof. In certain embodiments, L consists of one or more groups selected from amino acids, polyether chains, aliphatic groups, and any combinations thereof.

In certain embodiments, L is a covalent bond or a bivalent C₁₋₃₀ saturated or unsaturated, straight or branched, hydrocarbon chain, wherein 1-10 methylene units of L are optionally and independently replaced by cyclopropylene, —N(H)—, —N(C₁₋₄ alkyl)-, —N(C₃₋₅ cycloalkyl)-, —O—, —C(O)—, —S—, —SO—, or —SO₂—. In certain embodiments, L is a covalent bond.

In certain embodiments, the linker is a bivalent C₁₋₂₀ saturated or unsaturated, straight or branched, hydrocarbon chain, wherein 1-7 methylene units of L are optionally and independently replaced by —N(H)—, —N(C₁₋₄ alkyl)-, —O—, or —C(O)—. In certain embodiments, L is a bivalent C₅₋₁₅ saturated or unsaturated, straight or branched, hydrocarbon chain, wherein 1-7 methylene units of L are optionally and independently replaced by —N(H)—, —N(C₁₋₄ alkyl)-, —O—, or —C(O)—.

In certain embodiments, the linker comprises a polymer defined as L₁ having 1, 2, 3, 4, 5, 6, 7, 8, 9, 10, 11, or 12 homotypically- or heterotypically-repeating subunits selected among structures.

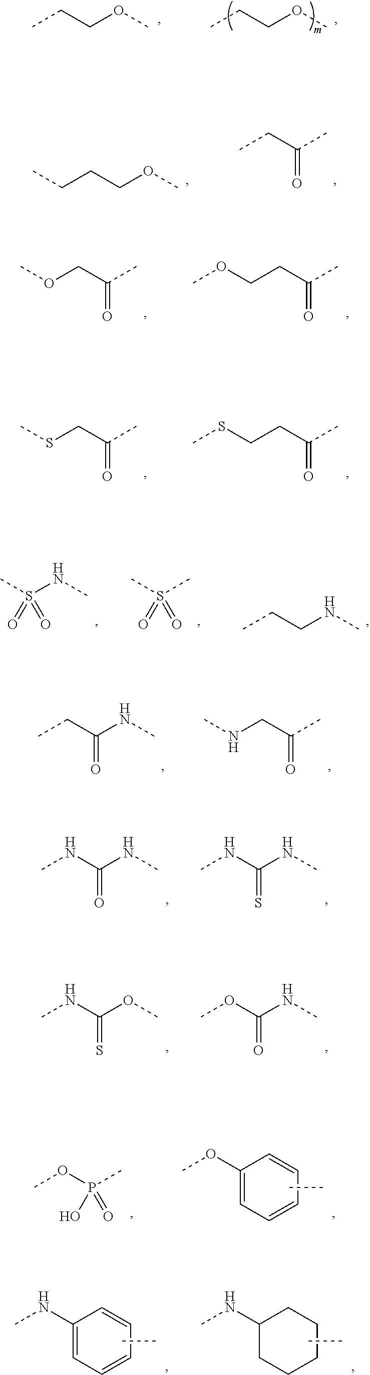

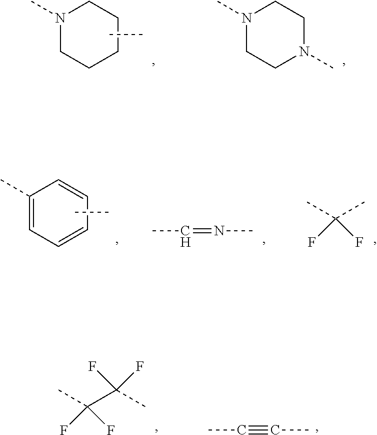

wherein a dashed bond indicates a point of attachment to another polymer unit, to a saturated or unsaturated alkyl chain, to a linear or branched alkyl chain, to a methylene, to an amino acid, to an oligopeptide, to [symbol] , to [symbol] , or to the rest of the compound, and m is a number between 1 and 12.

In certain embodiments, the linker (L) comprises a substituted “Click chemistry” product exemplified by any one of L₂ structures:

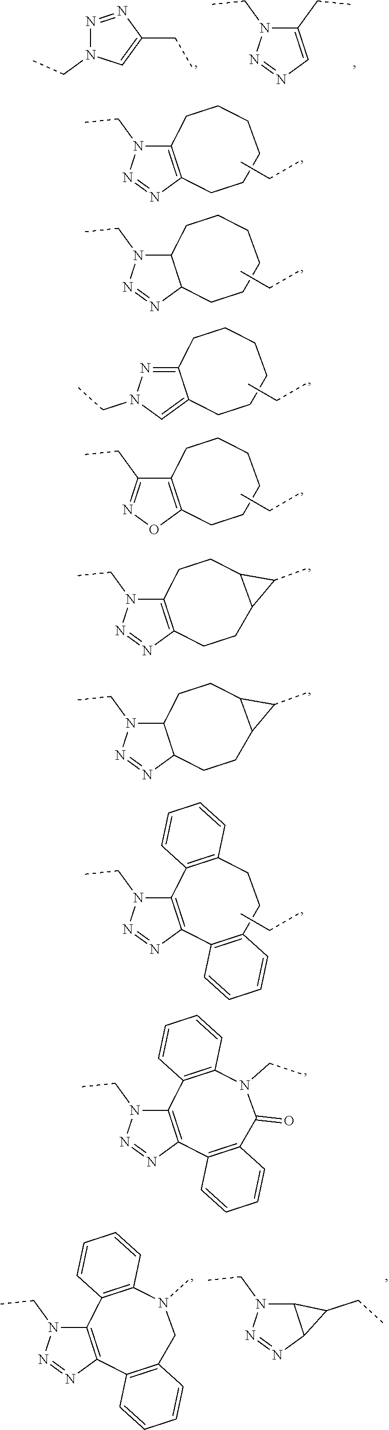

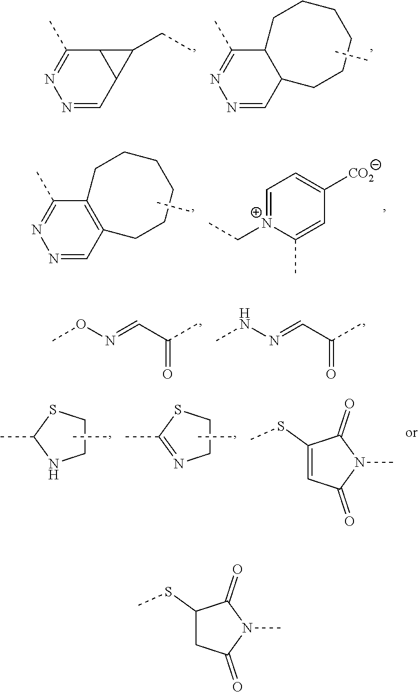

that may be part of larger structures where H atoms are substituted with fluorine, alkyl, phenyl, and aryl groups, wherein a dashed bond indicates a point of attachment to an L₁ structure, to a saturated or unsaturated alkyl chain, to a linear or branched alkyl chain, to a methylene, to an amino acid, to a peptide, to [symbol] or [symbol] .

In certain embodiments, the linker L is described by the formulas L₁-L₂, L₁-L₂-L₁′, L₁-L₁′-L₂, wherein L₁ and L₁′ are comprised of the same or of different polymer sub-unit arrangements.

In certain embodiments, L comprises a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units.

In certain embodiments, L is optionally substituted (poly)ethyleneglycol having between 1 and about 100 ethylene glycol units, between about 1 and about 50 ethylene glycol units, between 1 and about 25 ethylene glycol units, between about 1 and about 10 ethylene glycol units, between 1 and about 8 ethylene glycol units, between 1 and about 6 ethylene glycol units, between 2 and about 4 ethylene glycol units, or optionally substituted alkyl groups interdispersed with optionally substituted, O, N, S, P or Si atoms. In certain embodiments, L is substituted with an aryl, phenyl, benzyl, alkyl, alkylene, or heterocycle group.

In certain embodiments, L is -(A^{L})_{q}-, wherein:

q is an integer greater than or equal to 1 (e.g., 1, 2, 3, 4, 5, 6, 7, 8, 9 or 10);

each A^{L} is independently selected from the group consisting of a bond, CR^{L1}R^{L2}, O, S, SO, SO², NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4} NR^{L3}C(═NCN), NR^{L3}C(═CNO₂)NR^{L4}, C₃₋₁₁cycloalkyl optionally substituted with 0-6 RY and/or R^{L2} groups, C₅₋₁₃ spirocycloalkyl optionally substituted with 0-9 RY and/or R^{L2} groups, C₃₋₁₁heterocyclyl optionally substituted with 0-6 R^{L1} and/or R^{L2} groups, C₅₋₁₃ spiroheterocycloalkyl optionally substituted with 0-8 RY and/or R^{L2} groups, aryl optionally substituted with 0-6 RY and/or R^{L2} groups, heteroaryl optionally substituted with 0-6 RY and/or R^{L2} groups, where RY or R^{L2}, each independently are optionally linked to other groups to form cycloalkyl and/or heterocyclyl moiety, optionally substituted with 0-4 R^{L5} groups; and

R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CH₁F₂, CH₂F, NO₂, SFs, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NHSO₂NH(C₁₋₈alkyl), NHSO₂N(C₁₋₈alkyl)₂, or NHSO₂NH₂.

In some embodiments, q is 1 to 2. In some embodiments, q is 1 to 5. In some embodiments, q is 1 to 10. In some embodiments, q is 1 to 20. In some embodiments, q is 1, 2, 3, 4, 5, 6, 7, 8, 9, 10, 11, 12, 13, 14, 15, 16, 17, 18, 19, or 20. In some embodiments, q is an integer from 1 to 100, 1 to 90, 1 to 80, 1 to 70, 1 to 60, 1 to 50, 1 to 40, or 1 to 30.

In certain embodiments, L is a group of the formula:

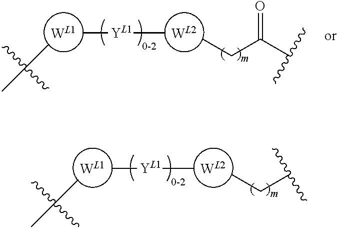

wherein:

the symbol “ [symbol] ” indicates a point of attachment to [symbol] or [symbol] ;

W^{L1} and W^{L2} are each independently a 4-8 membered ring with 0-4 heteroatoms, optionally substituted with R^{Q}; wherein each R^{Q} is independently a H, halo, OH, CN, CF₃, optionally substituted C₁-C₆ alkyl, optionally substituted C₁-C₆ alkoxy, or 2 R^{Q} groups are taken together with the atom they are attached to, form a 4-8 membered ring system containing 0-4 heteroatoms;

Y^{L1} is each independently a bond, optionally substituted C₁-C₆ alkyl, or optionally substituted 2-8 membered heteroalkyl (e.g., C₁-C₆ alkoxy; and

m is 0-10.

In certain embodiments, L is a group of the formula:

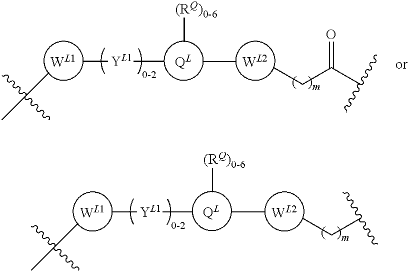

wherein:

the symbol “ [symbol] ” indicates a point of attachment to [symbol] or [symbol] ,

W^{L1} and W^{L2} are each independently aryl, heteroaryl, cyclic, heterocyclic, C₁₋₆ alkyl, bicyclic, biaryl, biheteroaryl, or biheterocyclic, each optionally substituted with R^{Q}; wherein each R^{Q} is independently a H, halo, OH, CN, CF₃, hydroxyl, nitro, C≡CH, C₂₋₆ alkenyl, C₂₋₆ alkynyl, optionally substituted C₁-C₆ alkyl, optionally substituted C₁-C₆ alkoxy, optionally substituted O—C₁₋₃alkyl (e.g., C₁₋₃ haloalkoxyl), OH, NH₂, NR^{Y1}R^{Y2}, CN, or 2 R^{Q} groups are taken together with the atom they are attached to, form a 4-8 membered ring system containing 0-4 heteroatoms;

Y^{L1} is each independently a bond, NR^{YL1}, O, S, NR^{YL2}, CR^{YL1}R^{YL2}, C═O, C═S, SO, SO₂, optionally substituted C₁-C₆ alkyl, or optionally substituted C₁-C₆ alkoxy;

Q^{L} is a 3-6 membered alicyclic or aromatic ring with 0-4 heteroatoms, optionally bridged, optionally substituted with 0-6 R^{Q}; wherein each R^{Q} is independently H, optionally substituted C₁₋₆ alkyl, optionally substituted C₁₋₆ alkoxyl, or 2 R^{Q} groups are taken together with the atom they are attached to, form a 3-8 membered ring system containing 0-2 heteroatoms);

RY^{L1}, RY^{L2} are each independently H, OH, optionally substituted C₁₋₆ alkyl, or R¹, R² together with the atom they are attached to, form a 3-8 membered ring system containing 0-2 heteroatoms); and

m is 0-10.

In some embodiments, L is selected from the group consisting of —NR(CH₂)ₘ-(lower alkyl)-, —NR(CH₂)ₘ-(lower alkoxyl)-, —NR(CH₂)ₘ-(lower alkoxyl)-OCH₂—, —NR(CH₂)ₘ-(lower alkoxyl)-(lower alkyl)-OCH₂—, —NR(CH₂)ₘ-(cycloalkyl)-(lower alkyl)-OCH₂—, —NR(CH₂)ₘ—, (hetero cycloalkyl)-, —NR(CH₂CH₂O)ₘ-(lower alkyl)-O—CH₂—, —NR(CH₂CH₂O)ₘ-(hetero cycloalkyl)-O—CH₂—, —NR(CH₂CH₂O)ₘ-Aryl-O—CH₂—, —NR(CH₂CH₂O)ₘ-(hetero aryl)-O—CH₂—, —NR(CH₂CH₂O)ₘ-(cyclo alkyl)-O-(hetero aryl)-O—CH₂—, —NR(CH₂CH₂O)ₘ-(cyclo alkyl)-O-Aryl-O—CH₂—, —NR(CH₂CH₂O)ₘ-(lower alkyl)-NH-Aryl-O—CH₂—, —NR(CH₂CH₂O)ₘ-(lower alkyl)-O-Aryl-CH₂, —NR(CH₂CH₂O)ₘ-cycloalkyl-O-Aryl-, —NR(CH₂CH₂O)ₘ-cycloalkyl-O-(heteroaryl), —NR(CH₂CH₂)ₘ-(cycloalkyl)-O-(heterocycle)-CH₂, —NR(CH₂CH₂)ₘ-(heterocycle)-(heterocycle)-CH₂, —N(R¹R²)-(heterocycle)-CH₂; wherein m of L can be 0 to 10; R of L can be H, lower alkyl; and R¹ and R² of L can form a ring with the connecting N.

The description above describes multiple embodiments relating to compounds of Formula I or II. The present disclosure specifically contemplates all combinations of the embodiments.

In some embodiments, the linker is represented by any one of structures:

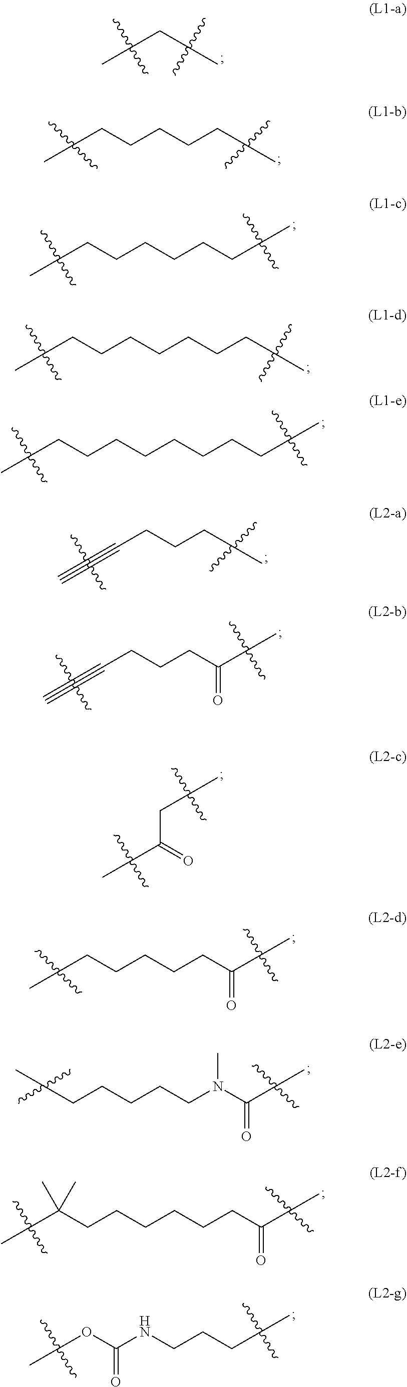

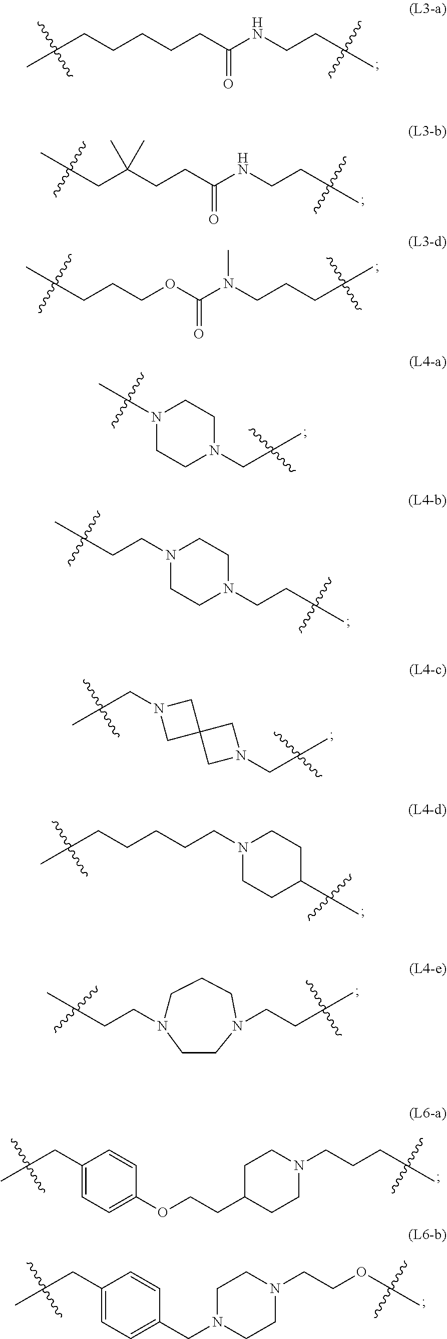

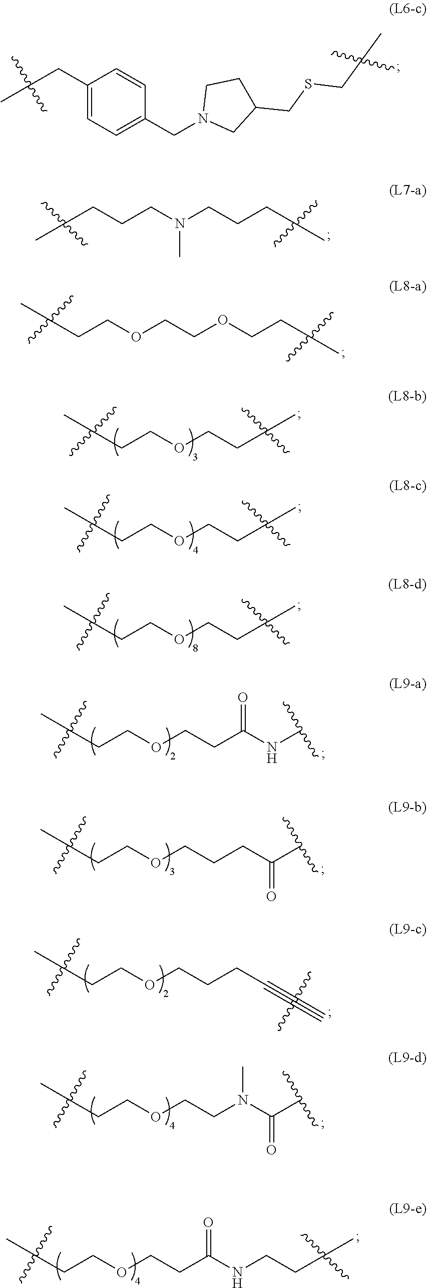

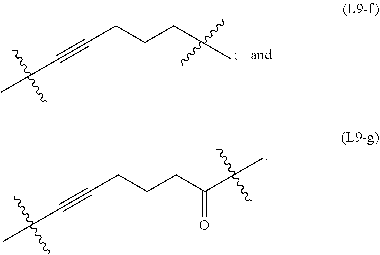 [SMILES: *C#CCCC* ; *C#CCCCC(*)=O]

Compounds disclosed herein may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present disclosure with a suitable acid or a base. Examples of pharmaceutically acceptable salts of the compounds of this disclosure include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the disclosure can form pharmaceutically acceptable salts with various organic bases such as lysine, arginine, guanidine, diethanolamine or metformin.

Compounds as disclosed herein may have at least one chiral center. Therefore, they may be in the form of a stereoisomer. As used herein, the term “stereoisomer” embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present disclosure may be made and used in the form of individual isomers and substantially free of other isomers, or in the form of a mixture of various isomers, e.g., racemic mixtures of stereoisomers.

In some embodiments, a compound disclosed herein is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

In addition, compounds of the present disclosure embrace N-oxides, crystalline forms (also known as polymorphs), active metabolites of the compounds having the same type of activity, tautomers, and unsolvated as well as solvated and hydrated forms with pharmaceutically acceptable solvents such as water, ethanol, and the like, of the compounds. The solvated forms of the conjugates presented herein are also considered to be disclosed herein.

### Methods of Synthesis

In some embodiments, the present disclosure is directed to methods for making a compound as disclosed herein or a pharmaceutically acceptable salts or stereoisomers thereof. Broadly, the inventive compounds or pharmaceutically-acceptable salts or stereoisomers thereof, may be prepared by any process known to be applicable to the preparation of chemically related compounds. The compounds disclosed herein will be better understood in connection with the synthetic schemes described in various working examples that illustrate non-limiting methods by which the compounds of the disclosure may be prepared.

### Methods of Generating Masked-Warhead and Warhead-Bearing DEL Compounds

In some aspects, the present disclosure is directed to methods of creating an electrophilic warhead-bearing DNA-Encoded Library (eDEL) comprising:

coupling the compound of formula I or II with a DNA-Encoded Library to generate a stable masked-warhead DEL (mwDEL) intermediate;

purifying the mwDEL intermediate;

unmasking the mwDEL intermediate to generate an activated eDEL; and

purifying the activated eDEL.

In some embodiments, the coupling of the compound of formula I with a DEL is achieved via an amide coupling reaction (Shi et al., RSC Adv. 11(4):2359-2376 (2021), Gartner et al., Angew. Chem. Int. Ed., 41:1796-1800 (2002)).

In some embodiments, the coupling of the compound of formula I with a DEL is proceeded by base pairing interactions between the DEL barcode and an oligonucleotide tag as described in Gartner et al., Angew. Chem Int. Ed. 42(12):1370-1375 (2003), Gartner et al., Science 305:1601-1605 (2004), Tse et al., J. Am. Chem. Soc. 130:15611-15626 (2008), and Usanov et al., Nat. Chem. 10(7):704-714 (2018). Base pairing interactions between a DEL barcode and an oligonucleotide tag are also described in International Patent Publication No WO 2019/168654, U.S. Patent Application Publication Nos 2006/223086, 2005/0170376, and 2009/003582, and U.S. Pat. No. 7,479,472, each of which is incorporated herein by reference.

### Methods of Generating Masked-Warhead and Warhead-Bearing DEL Compounds

The present disclosure is directed to a method in which the masked warhead-bearing DEL intermediate comprises an arylsulfone.

In some embodiments, the present disclosure is directed to a method in which the masked warhead-bearing DEL intermediate is a Julia-Kocienski reaction intermediate.

In some embodiments, the present disclosure is directed to a method in which the masked warhead-bearing DEL intermediate is a Julia-Kocienski reaction intermediate, wherein the hydroxyl group is available for elimination. In some embodiments, the hydroxyl group is eliminated through a Smiles rearrangement (Levy et al., J. Chem. Soc., 1931, 3264-3269). In some embodiments, the hydroxyl group is eliminated through a beta elimination reaction (Clayden J; Greeves N; Warren S Organic chemistry. 2nd Edition ed.; Oxford University Press: New York, 201).

In some embodiments, the present disclosure is directed to a method in which the masked warhead-bearing DEL intermediate is a Julia-Kocienski reaction intermediate, wherein the hydroxyl group is attached to a Protecting Group (PG).

In some embodiments, the present disclosure is directed to a method in which the stable masked-warhead DEL intermediate is purified via gel electrophoresis purification.

In some embodiments, the present disclosure is directed to a purification method that involves polyacrylamide gel electrophoresis.

In some embodiments, the present disclosure is directed to a purification method that involves agarose gel electrophoresis.

In some embodiments, the present disclosure is directed to a purification method that involves capillary gel electrophoresis.

In some embodiments, the present disclosure is directed to a method in which the stable masked-warhead mwDEL intermediate is purified via chromatographic separation.

In some embodiments, the present disclosure is directed to a purification method that involves liquid chromatography (LC).

In some embodiments, the present disclosure is directed to a purification method that involves size-exclusion chromatography (SEC).

In some embodiments, the present disclosure is directed to a purification method that involves ion exchange chromatography (e.g., cation- or anion-exchange chromatography).

In some embodiments, the present disclosure is directed to a method in which the stable masked-warhead DEL intermediate is purified via precipitation (e.g., ethanol precipitation).

In some embodiments, the present disclosure is directed to a method in which the stable masked-warhead DEL intermediate is purified via affinity-based methods.

In some embodiments, the present disclosure is directed to a purification method that involves an avidin protein.

In some embodiments, the present disclosure is directed to a purification method that involves oligonucleotide base-pairing.

In some embodiments, the present disclosure is directed to a purification method that involves antibodies (e.g. anti-HA, anti-Flag, anti-Myc, etc.).

In some embodiments, two or more purification methods are applied in series.

Other purification methods that may be used are described in Usanov et al., Nat. Chem. 10(7):704-714 (2018) and in International Patent Publication WO 2019/168654, which is incorporated herein by reference.

In some embodiments, the purified mwDEL intermediate is storable.

In some embodiments, the purified mwDEL intermediate is storable at a temperature between −150° C. and 25° C.

In some embodiments, the purified mwDEL intermediate is storable at a temperature between 25° C. and 40° C. (room temperature).

In some embodiments, the purified mwDEL intermediate is storable at a temperature between 4° C. and room temperature.

In some embodiments, the purified mwDEL intermediate is storable at a temperature between 0° C. and 4° C.

In some embodiments, the purified mwDEL intermediate is storable at a temperature between −20° C. and room 0° C.

In some embodiments, the purified mwDEL intermediate is storable at a temperature between −40° C. and room −20° C.

In some embodiments, the purified mwDEL intermediate is storable at a temperature between −78° C. and room −40° C.

In some embodiments, the purified mwDEL intermediate is storable at a temperature between −82° C. and room −78° C.

In some embodiments, the purified mwDEL intermediate is storable at a temperature between −150° C. and room −80° C.

In some embodiments, the purified mwDEL intermediate is storable in the presence of oxygen.

In some embodiments, the purified mwDEL intermediate is storable in the presence of water.

In some embodiments, the purified mwDEL intermediate is storable in the presence of organic solvents.

In some embodiments, the purified mwDEL intermediate is storable in the presence of dimethyl sulfoxide.

In some embodiments, the purified mwDEL intermediate is storable for one day to up to two or more years.

In some embodiments, the purified mwDEL intermediate is storable for one day.

In some embodiments, the purified mwDEL intermediate is storable for two or more days.

In some embodiments, the purified mwDEL intermediate is storable for one week.

In some embodiments, the purified mwDEL intermediate is storable for two or more weeks.

In some embodiments, the purified mwDEL intermediate is storable for one month.

In some embodiments, the purified mwDEL intermediate is storable for two or more months.

In some embodiments, the purified mwDEL intermediate is storable for one year.

In some embodiments, the purified mwDEL intermediate is storable for two or more years.

In some embodiments, an amide coupling reagent activates the carboxylic acid group of formula (I) or II for amide bond formation with a DEL.

In some embodiments, the amide coupling reagent that activates the carboxylic acid group of formula I or II is 1-ethyl-3-(3′-dimethyl-aminopropyl)carbodiimide (EDC); N,N′-dicyclohexylcarbodiimide (DCC); N,N′-diisopropylcarbodiimide (DIC); 1-[bis(dimethylamino)methylene]-1H-1,2,3-triazolo[4,5-b]pyridinium 3-oxide hexafluorophosphate (HATU); 2-(1H-benzotriazole-1-yl)-1,1,3,3-tetramethyluronium hexafluorophosphate (HBTU); benzotriazol-1-yloxytripyrrolidinophosphonium hexafluorophosphate (PyBOP); bromo-tris-pyrrolidino-phosphonium hexafluorophosphate (PyBroP); (1-cyano-2-ethoxy-2-oxoethylidenaminooxy)dimethylamino-morpholino-carbenium hexafluorophosphate (COMU); N-methyl-2-chloropyridinium tetrafluoroborate; ethyl 2-cyano-2-(hydroxyimino)acetate (Oxyma); 4-(4,6-dimethoxy-1,3,5-triazin-2-yl)-4-methyl-morpholinium chloride (DMTMM); N-hydroxysuccinimide (NHS); N-hydroxysulfosuccinimide (sulfo-NHS); 1-hydroxybenzotriazole (HOBt); 1-hydroxy-7-azabenzotriazole (HOAt); or 3-hydroxy-1,2,3-benzotriazin-4(3H)-one (DhbtOH).

In some embodiments, the mwDEL unmasking involves the use of an acidic aqueous solution.

In some embodiments, the mwDEL unmasking involves the use of a reducing aqueous solution.

In some embodiments, the mwDEL unmasking involves the use of an oxidizing aqueous solution.

In some embodiments, the mwDEL unmasking involves the use of an alkaline aqueous solution.

In some embodiments, the alkaline solution comprises Tris buffer, phosphate buffer, acetate buffer, ammonium hydroxide buffer, dimethylamine buffer, methylamine buffer, citrate buffer, N-(2-hydroxyethyl)piperazine-N-(2-ethanesulfonic acid) (HEPES), 2-(N-Morpholino) ethanesulfonic acid (MES), 3-(N-Morpholino) propanesulfonic acid (MOPS), or N-tris hydroxymethyl methyl-3-aminopropanesulfonic acid (TAPS), 4-morpholinebutanesulfonic acid (MOBS), 3-morpholino-2-hydroxypropanesulfonic acid (MOPSO), (4-(2-hydroxyethyl)-1-piperazineethanesulfonic acid) (HEPES), 3-[4-(2-hydroxyethyl)-1-piperazinyl propanesulfonic acid (EPPS or HEPPS), piperazine-N,N-bis(2-ethanesulfonic acid) (PIPES), N-(2-hydroxyethyl)piperazine-N-(2-hydroxypropanesulfonic acid) (HEPPSO); N-(2-hydroxyethyl)piperazine-N-(4-butanesulfonic acid) (HEPBS), N-cyclohexyl-2-aminoethanesulfonic acid (CHES), N-cyclohexyl-3-aminopropanesulfonic acid (CAPS), 4-(cyclohexylamino)-1-butanesulfonic acid (CABS), 3-(cyclohexylamino)-2-hydroxyl-1-propanesulfonic acid (CAPSO), N,N-bis(2-hydroxyethyl)-2-aminoethanesulfonic acid (BES), bis-(2-hydroxyethyl)amino-tris(hydroxymethyl)methane (BisTris), bis(2-hydroxyethyl) aminotris(hydroxymethyl) methane (Bicine), piperazine-N,N′-Bis[2-hydroxypropanesulfonic acid] (Tricine), or N,N-bis(2-hydroxyethyl)-3-amino-2-hydroxypropanesulfonic acid (DIPSO).

In some embodiments, the mwDEL unmasking involves the use of an inorganic hydroxide (OH⁻) base, such as alkali hydroxides, (e.g., sodium hydroxide, potassium hydroxide), alkaline earth metal hydroxides, (e.g., barium hydroxide), or alkali metal or alkaline earth metal alkoxides, (e.g., sodium methoxide, potassium methoxide, sodium ethoxide, potassium ethoxide, potassium tert-butoxide, etc.).

In some embodiments, the mwDEL unmasking involves the use of an inorganic carbonate base, such as alkali metal carbonates (e.g., sodium carbonate, potassium carbonate or caesium carbonate) or alkaline earth metal carbonates (e.g., calcium carbonate, etc.).

In some embodiments, the mwDEL unmasking involves the use of an organic base or amino base (trialkyl(C₁-C₆)amines), such as triethylamine, (dialkyl(C₁-C₆)amines), such as dimethylamine, (alkyl(C₁-C₆)amines) such as methylamine, ammonia, hydrazines, phosphazenes, or heterocycles, such as 1,4-diazabicyclo[2.2.2]octane (DABCO), 1,8-diazabicyclo[5.4.0]undec-7-ene (DBU), 1,5-diazabicyclo[4.3.0]non-5-ene (DBN), pyridine, diaminopyridine, 4-dimethylaminopyridine (DMAP), methylpiperidine, or morpholine.

In some embodiments, the mwDEL unmasking involves the use of a hydroxide (OH⁻) salt solution.

In some embodiments, the mwDEL unmasking involves the use of a base with pKa >14.

In some embodiments, the mwDEL unmasking reaction is promoted by a solution of pH less than 7.

In some embodiments, the mwDEL unmasking reaction is promoted by a solution of pH 7 to 8.

In some embodiments, the mwDEL unmasking reaction is promoted by a solution of pH 8 to 9.

In some embodiments, the mwDEL unmasking reaction is promoted by a solution of pH 9 to 10.

In some embodiments, the mwDEL unmasking reaction is promoted by a solution of pH 10 to 11.

In some embodiments, the mwDEL unmasking reaction is promoted by a solution of pH 11 to 12.

In some embodiments, the mwDEL unmasking reaction is promoted by a solution of pH more than 12.

In some embodiments, the activated eDEL comprises acrylamide moieties from the unmasking reaction.

### Methods of Use

In some aspects, the present disclosure is directed to an eDEL generated from the methods described above and methods of using the same.

In some embodiments, the eDEL is used in an in vitro selection assay followed by DNA sequencing. In some embodiments, the in vitro selection assay comprises screening protein ligands.

In some embodiments, the in vitro selection assay comprises screening ligands that covalently modify a residue of a protein.

In some embodiments, the in vitro selection assay comprises screening ligands that covalently modify the thiol group of a Cysteine residue of a protein.

In some embodiments, the in vitro selection assay comprises screening ligands that covalently modify the imidazole ring of a Histidine residue of a protein.

In some embodiments, the in vitro selection assay comprises screening ligands that covalently modify the amino group of a Lysine residue of a protein.

In some embodiments, the in vitro selection assay comprises screening ligands that covalently modify the hydroxyl group of a Serine residue of a protein.

In some embodiments, the in vitro selection assay comprises screening ligands that covalently modify the hydroxyl group of a Threonine residue of a protein.

In some embodiments, the in vitro selection assay comprises screening ligands that covalently modify the phenolic hydroxyl of a Tyrosine residue of a protein.

In some embodiments, the in vitro selection assay comprises screening ligands that covalently modify a carboxylate group of a protein.

In some embodiments, the in vitro selection assay comprises screening ligands that covalently modify an amide group of a protein.

In some embodiments, the in vitro selection assay comprises screening ligands that covalently modify an amino group of a protein.

In some embodiments, the in vitro selection assay comprises screening ligands that covalently modify a residue in the active site of a protein.

In some embodiments, the in vitro selection assay comprises screening ligands that covalently modify a residue in a non-orthosteric site of a protein.

In some embodiments, the in vitro selection assay comprises screening ligands that covalently modify a residue in an allosteric site of a protein.

In some embodiments, the in vitro selection assay comprises screening ligands that covalently modify a residue in a non-catalytic domain of a protein.

The present disclosure also provides uses of the eDELs in the in vitro selection assays described herein.

In some embodiments, the active site (e.g., the orthosteric site, or the catalytic site) is a pocket of a protein comprising amino acids that participate in the catalysis of a chemical reaction (e.g., by H-bonding, acid/base catalysis, transition-state stabilization, proximity, water activation, etc.), the binding interactions with an enzymatic cofactor (e.g., ATP, GTP, NADH, heme, vitamins, Zn, Mg, Ca, redox-active metals, etc.), or the binding interactions with a substrate (e.g., DNA, RNA, peptide, protein, metabolite, cofactor, vitamins, etc.) (Jacobson et al., Trends Biochem Sci 39(8):363-371 (2014); Izidoro et al., Bioinformatics 31(6):864-870 (2015); Feehan et al., Nat Commun. 12(1):3712 (2021)). In some embodiments, the active site comprises the binding pocket defined by nearby amino acids that are within at least about 2, 3, 5, 10, 15, or 20 Å from those amino acids that participate in a chemical reaction, cofactor, or substrate binding.

In some embodiments, the non-orthosteric site is a region of a protein distinct from the active site, which has defined or undefined functions that are accessory or complementary to the active site (e.g., protein-protein interactions, homodimerization, heterodimerization, homo-oligomerization, hetero-oligomerization, protein-DNA interactions, subcellular localization, compartmentalization, protein stability, protein destabilization, acceptor for ubiquitination, acceptor for phosphorylation, acceptor for acetylation, modulation of ternary structure, domain-domain contacts, substrate recruitment, conformational changes, etc.). In some embodiments, the non-orthosteric site comprises a binding pocket defined by amino acids that are at least about 5, 10, 15, or 20 Å away from the amino acids that participate in a chemical reaction, cofactor, or substrate binding. Examples of non-orthosteric sites include pockets in domains such as SH2, PTB, SH3, WW, WD40, PDZ, PH, RING, HECT, UBA, Tudor, CHROMO, BROMO, BIR, TRAF, CARD, pseudokinases, kinase C-lobe, kinase N-lobe pockets, among others (Berdasco et al., Nat Rev Genet, 20(2):109-127 (2019); Scott et al., Nat Rev Drug Discov. 15(8):533-550 (2016); Jin et al., Sci Signal., 2(98):ra76 (2009); Stanton et al., Science 359(6380):eaao5902 (2018)).

In some embodiments, the allosteric site is a type of non-orthosteric pocket that has defined or undefined functions that are accessory or complementary to the active site by imparting conformational changes to the protein upon the binding of a ligand, substrate, ion, protein partner, or cofactor (e.g., changes in secondary structure, changes in ternary structure, conformational activation, conformational inhibition, transition-state destabilization, change in substrate affinity, etc.) (Changeux et al., Nat Rev Mol Cell Biol, 14(12):819-829 (2013); Ubersax et al., Nat Rev Mol Cell Biol. 8(7):530-541 (2007)). In some aspects, the allosteric site imparts conformational changes within at least about 2, 3, 5, 10, 15, or 20 Å from those amino acids that participate in a chemical reaction, cofactor, or substrate binding.

In some embodiments, the non-catalytic domain of a protein is distinct from the region comprising the active site (alternatively, the protein has no active site) and this domain has functions that do not immediately produce changes to the chemical structure or composition of a substrate (e.g., proline amide bond isomerization, phospholipid bilayer organization, membrane binding, membrane fusion, membrane translocation, force, movement, switch behavior, sensors, channels, transporters, antiporters, translocation, storage, proximity, recruitment, protein-protein interactions, protein-DNA interactions, homodimerization, heterodimerization, homo-oligomerization, hetero-oligomerization, subcellular localization, compartmentalization, scaffolding, protein stability, protein destabilization, acceptor for ubiquitination, acceptor for phosphorylation, acceptor for acetylation, modulation of ternary structure, domain-domain contacts, substrate recruitment to a multi-protein complex, etc.). Examples of non-catalytic domains and non-catalytic proteins include proline isomerases, pseudokinases, chaperones, ion channels, voltage-gated ion channels, scaffold proteins, SNAREs, motor proteins, amyloid fibrils, phospholipid flippases, GTPase exchange factors (GEFs), extracellular receptors, transcription factors, ATPases, epigenetic mark readers, and non-orthosteric domains exemplified above, among others (Berdasco et al., Nat Rev Genet, 20(2):109-127 (2019); Scott et al., Nat Rev Drug Discov. 15(8):533-550 (2016); Jin et al., Sci Signal., 2(98):ra76 (2009)).

Methods of screening for covalent ligands using DELs are known in the art, for example, Chan, et al., Curr. Opin. Chem. Biol. 26:55-61 (2015), and Zhu et al., SLAS Discov. Adv. Sci. Drug Discov. 24(2):169-174 (2019)). The methods of screening for covalent ligands involve the incubation of a warhead-bearing DEL with a target protein, followed by: solid-supported separation of the protein and protein-bound DEL library members for Polymerase chain reaction (PCR) amplification and DNA sequencing analysis; or alternatively, interaction-dependent PCR and DNA sequencing analysis (Chan et al., J. Am. Chem. Soc. 139(30):10192-10195 (2017); Gorin et al., J. Am. Chem. Soc. 131:9189-9191 (2009)).

Additional methods of screening for covalent ligands using DELs are described in Guilinger et al., Bioorg. Med. Chem. 42:116223 (2021), Zimmermann et al., Chem. Eur. J. 23(34):8152-8155 (2017), Zambaldo, et al., MedChemComm 7(7):1340-1351 (2016), and Cochrane, et al., ACS Comb. Sci. 21(5):425-435 (2019).

### EXAMPLES

These and other aspects of the present disclosure will be further appreciated upon consideration of the following Examples, which are intended to illustrate certain particular embodiments of the disclosure but are not intended to limit its scope, as defined by the claims.

| Abbreviation | Description |
|---|---|
| Ac | acetyl |
| aq | aqueous |
| Bt | benzothiazole |
| DAST-F or | diethylaminosulfur trifluoride |
| DAST | |
| DBU | l,8-diazabicyclo[5.4.0]undec-7-ene |
| DCM | dichloromethane |
| DEL | DNA-encoded library of small molecules |
| DIPEA | N,N-diisopropylethylamine |
| DMF | dimethylformamide |
| DNA | deoxyribonucleic acid |
| DPPA | diphenylphosphoryl azide |
| EDC | l-ethyl-3-(3′-dimethyl-aminopropyl)carbodiimide |
| eDEL | electrophilic DEL / electrophilic |
| EGFR | epidermal growth factor receptor |
| Et | ethyl |
| EA | ethyl acetate |
| HA | hemagglutinin antigen |
| HATU | 1-[bis(dimethylamino)methylene]-1H- |
| | 1,2,3-triazolo[4,5- |
| | b]pyridinium 3-oxide hexafluorophosphate |
| HMDS | hexamethyldisilazane |
| HOAt | 1-hydroxy-7-azabenzotriazole |
| HOBt | 1-hydroxybenzotri azole |
| HPLC | high performance column chromatography |
| i-Pr | isopropyl |
| LAH | lithium aluminum hydride |
| LC | liquid chromatography |
| LCMS | liquid chromatography-mass spectrometry |
| LG | leaving group |
| LiHDMS | lithium hexamethyldisilazane |
| mCPBA | meta-chloroperoxybenzoic acid |
| Me | methyl |
| mwDEL | masked-warhead DEL |
| n-BuLi | n-butyllithium |
| oligo | oligonucleotide |
| Oxyma | ethyl cyanohydroxyiminoacetate |
| PAGE | polyacrylamide gel electrophoresis |
| PE | petroleum ether |
| PCR | polymerase chain reaction |
| PG | protecting group |
| Ph | phenyl |
| RT | room temperature |
| SEC | size-exclusion chromatography |
| TEA | trimethylamine |
| TFA | trifluoroacetic acid |
| THF | tetrahydrofuran |
| TIPS | triisopropylsilane |
| Ts | toluenesulfonyl |

### Example 1. Synthesis of Benzothiazole Sulfone DNA-Tagged Masked-Warhead Reagents

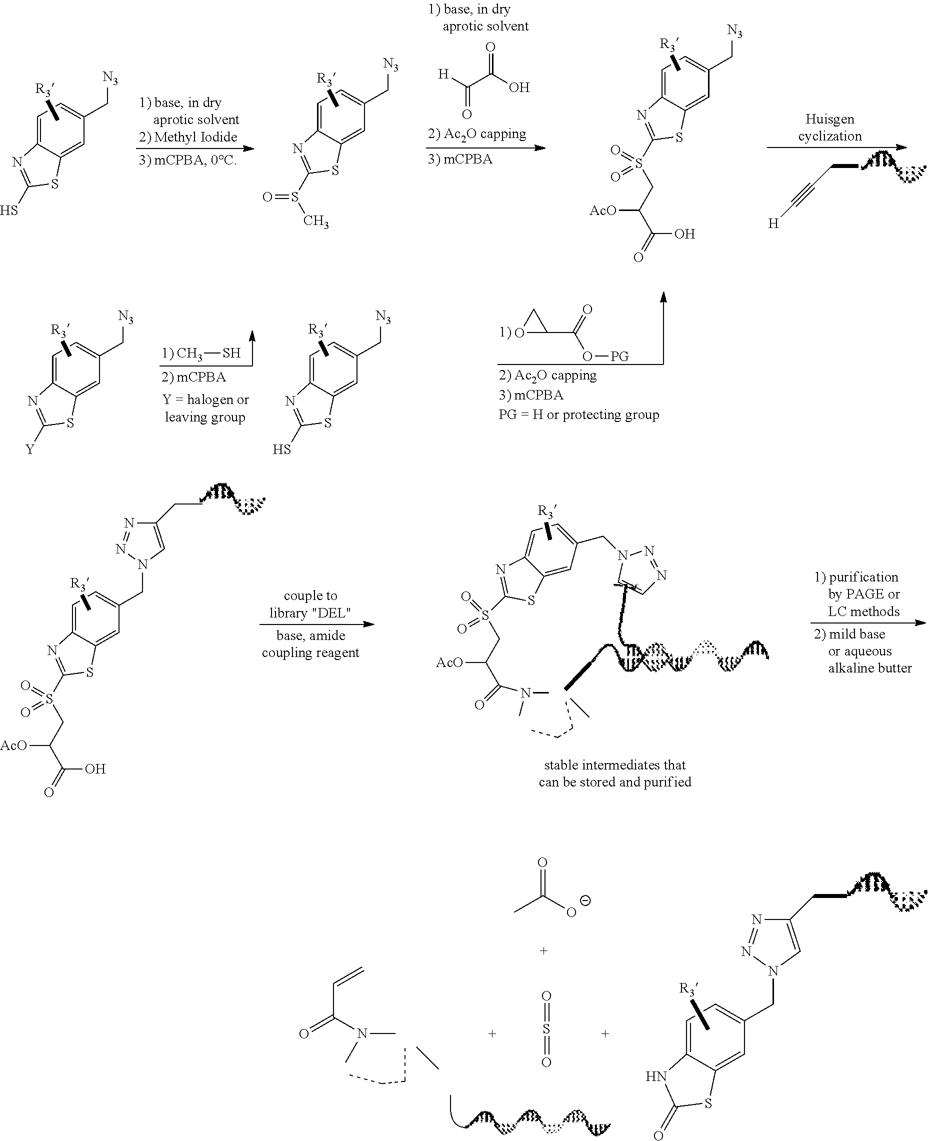

Benzothiazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 2. Synthesis of Benzothiazole Sulfone DNA-Tagged Masked-Warhead Reagents

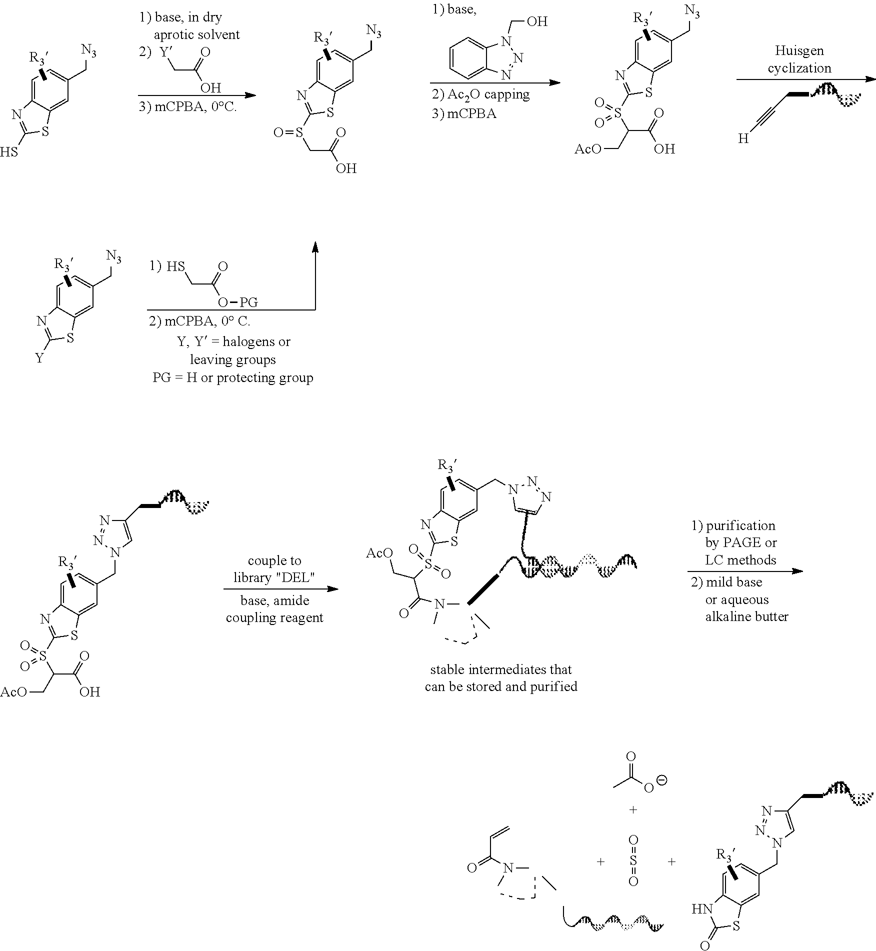

Benzothiazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 3. Synthesis of Benzothiazole Sulfone Biotin-Tagged Masked-Warhead Reagents

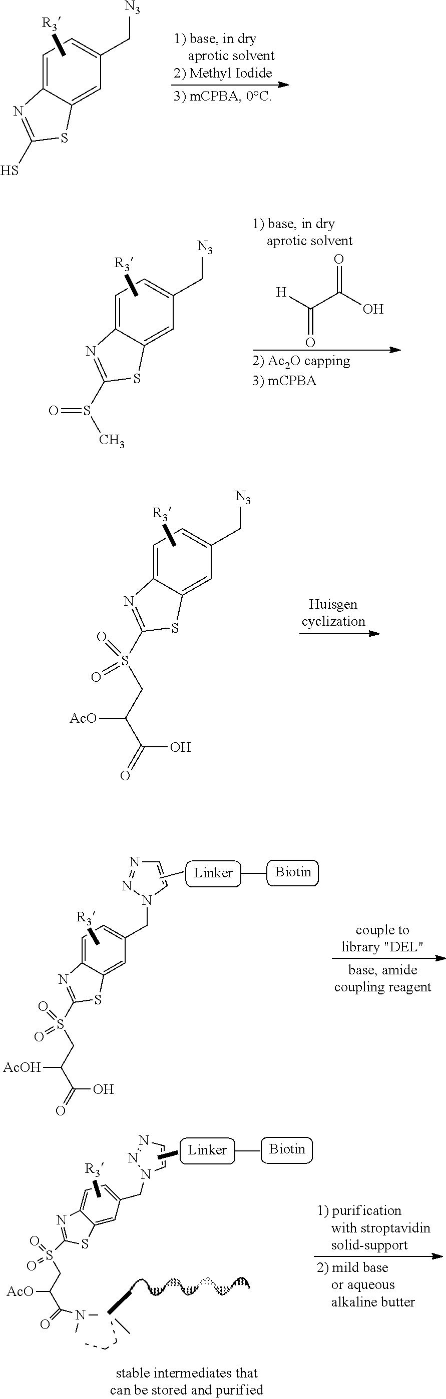

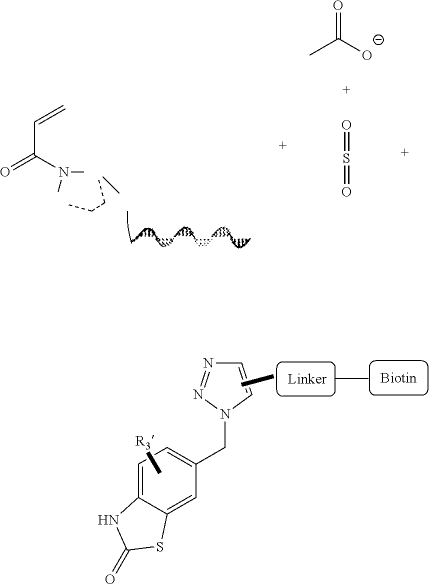

Benzothiazole sulfone biotin-tagged masked-warhead reagents are synthesized as illustrated in the above scheme. The reactions are performed as described The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 4. Synthesis of Benzothiazole Sulfone Biotin-Tagged Masked-Warhead Reagents

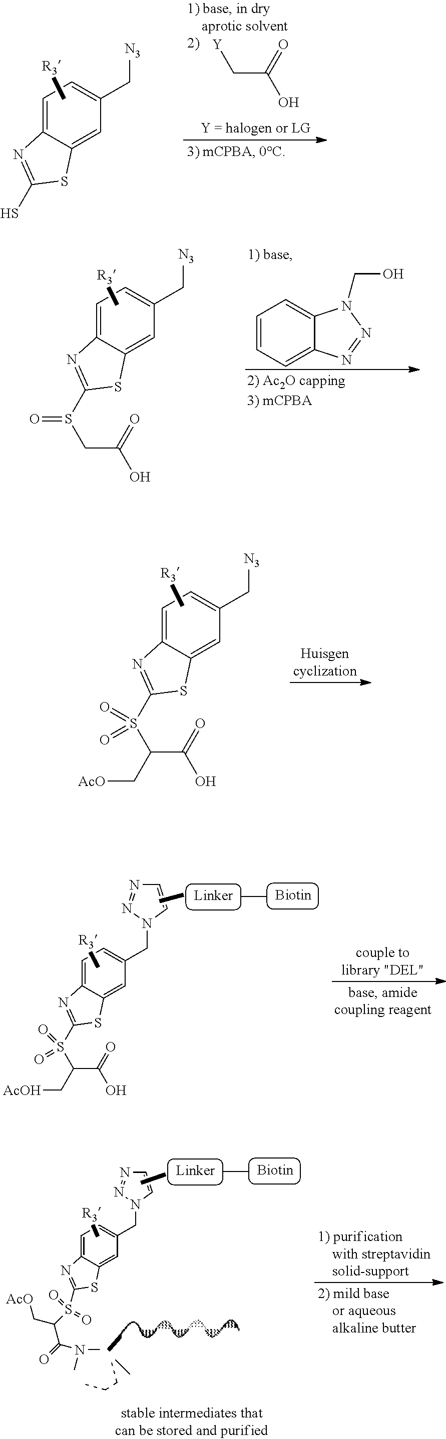

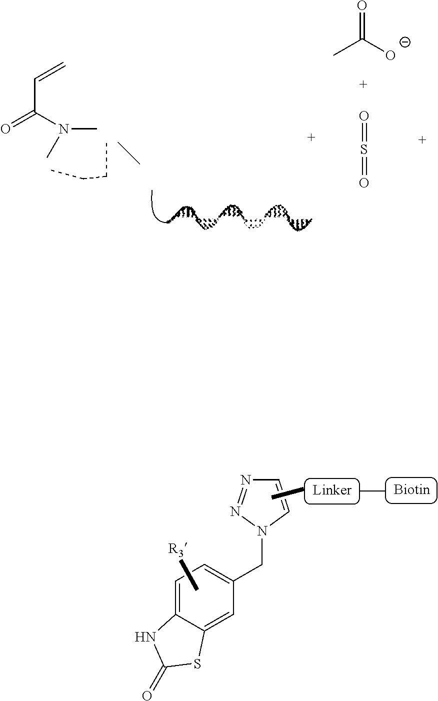

Benzothiazole sulfone biotin-tagged masked-warhead reagents are synthesized as illustrated in the above scheme. The reactions are performed as described in Blakemore et al., The Julia-Kocienski Olefination, In Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 5. Synthesis of 1-Alkyl-1H-Tetrazole-5-yl Sulfone DNA-Tagged Masked-Warhead Reagents

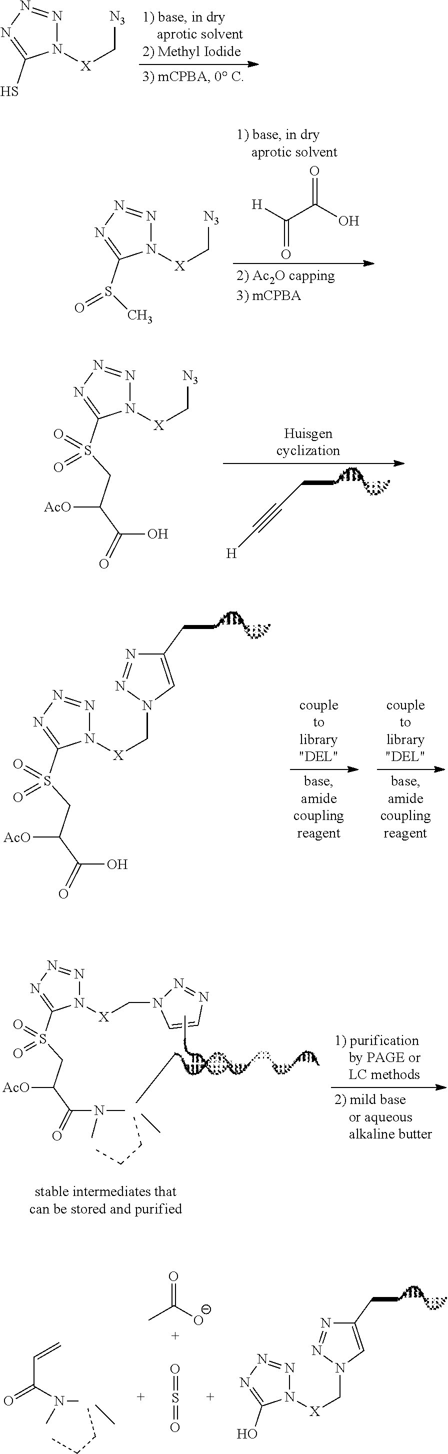

Alkytetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 6. Synthesis of 1-Phenyl-1H-Tetrazole-5-yl Sulfone DNA-Tagged Masked-Warhead Reagents

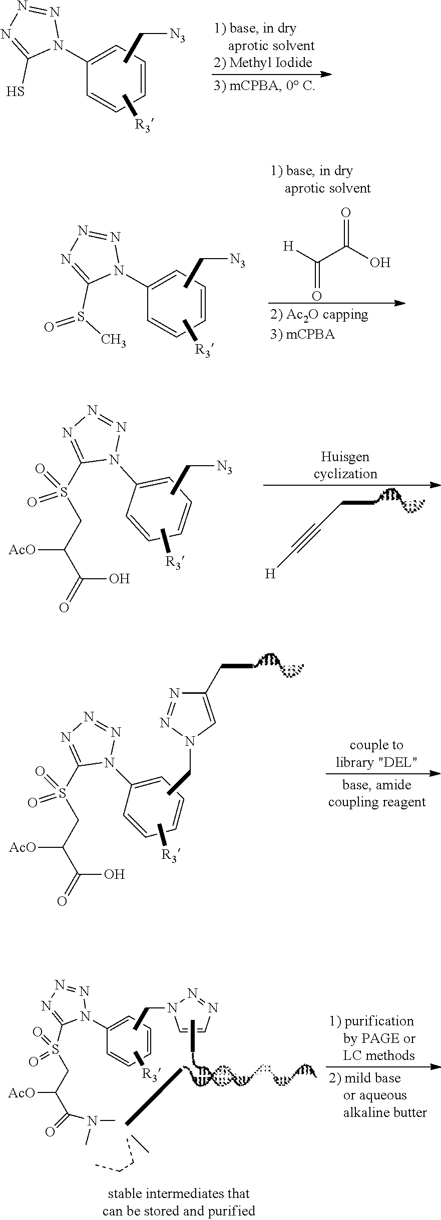

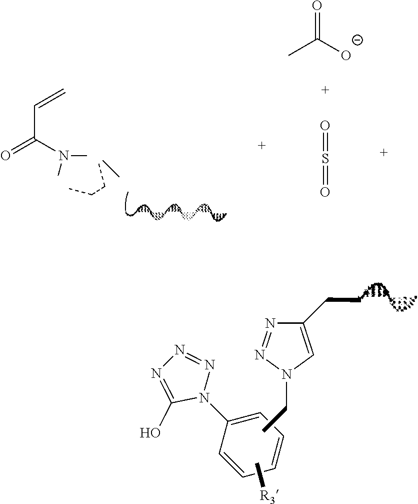

Phenyltetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 7. Synthesis of Pyrimidine-2-yl Sulfone DNA-Tagged Masked-Warhead Reagents

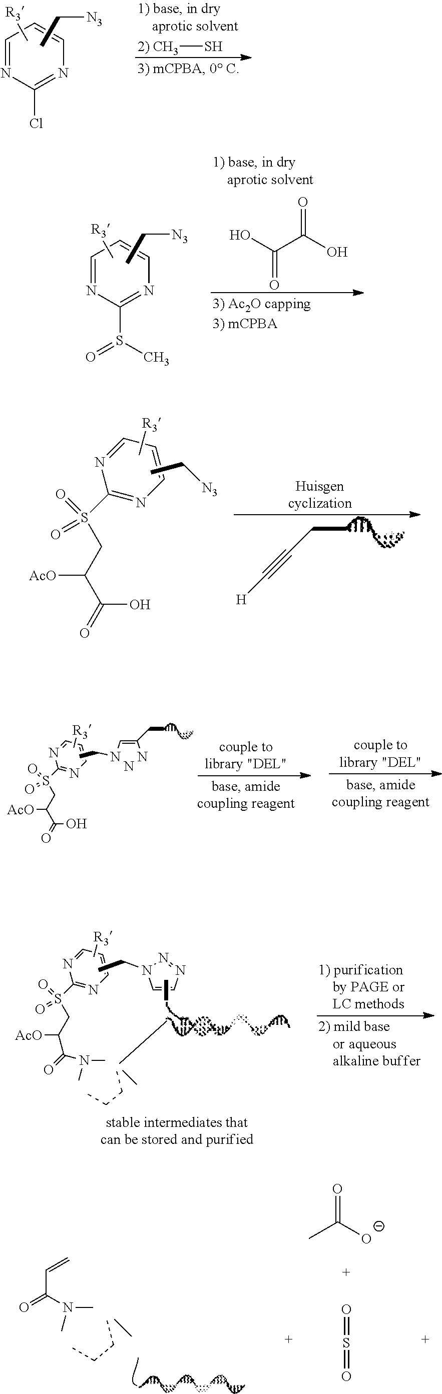

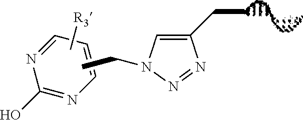

Pyrimidyl sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 8. Synthesis of 1-Alkyl-1H-Tetrazole-5-yl Sulfone DNA-Tagged Masked-Warhead Reagents

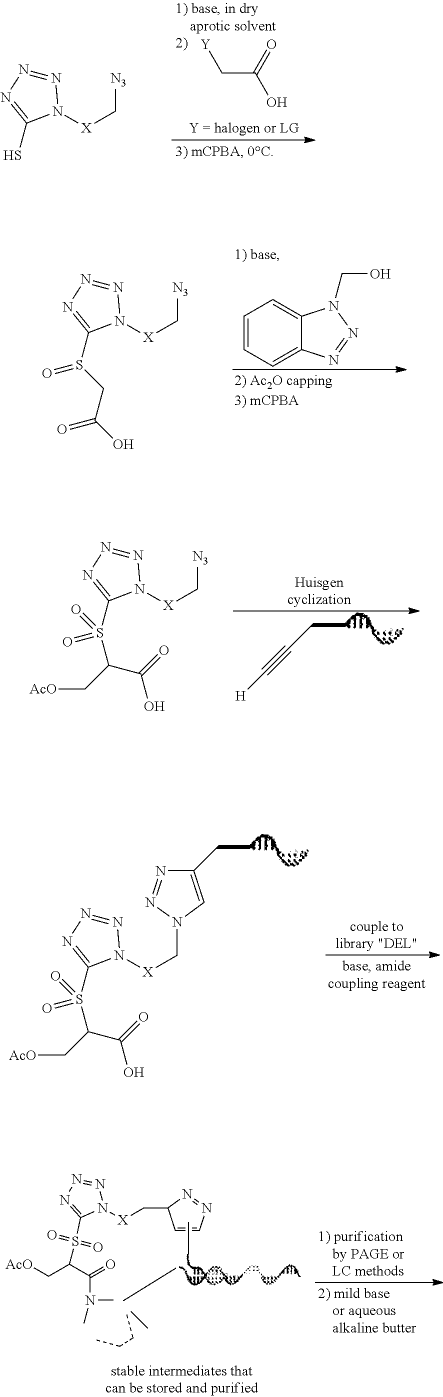

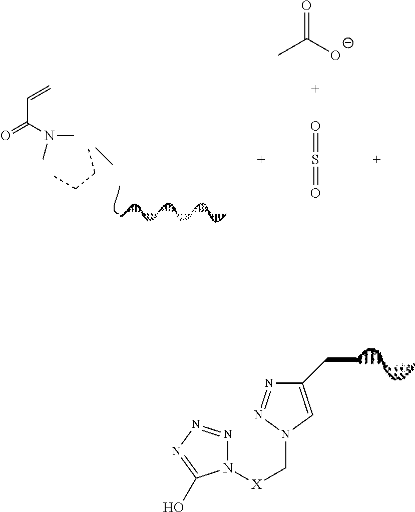

Alkytetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 9. Synthesis of 1-Phenyl-1H-Tetrazole-5-yl Sulfone DNA-Tagged Masked-Warhead Reagents

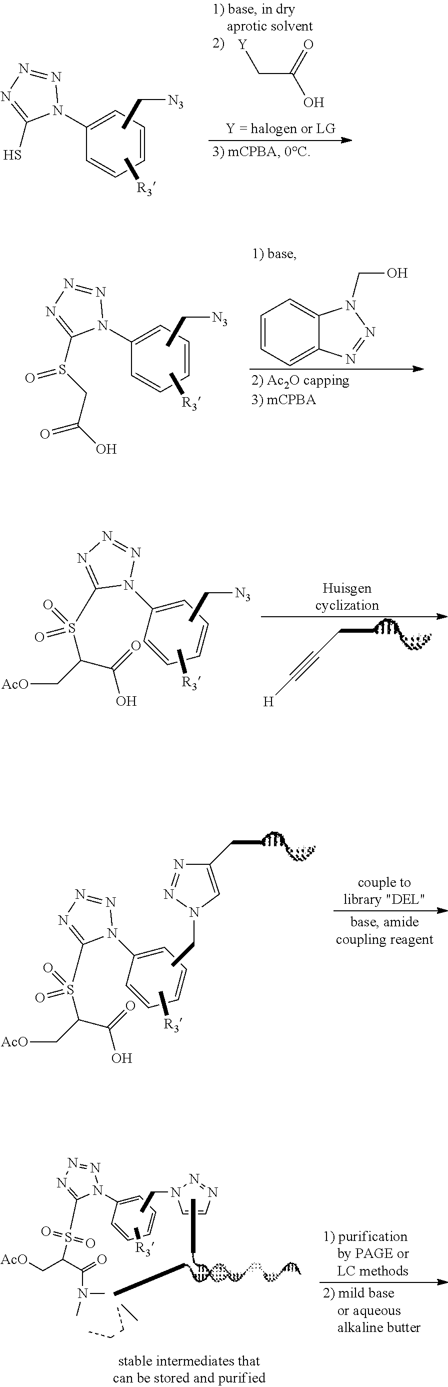

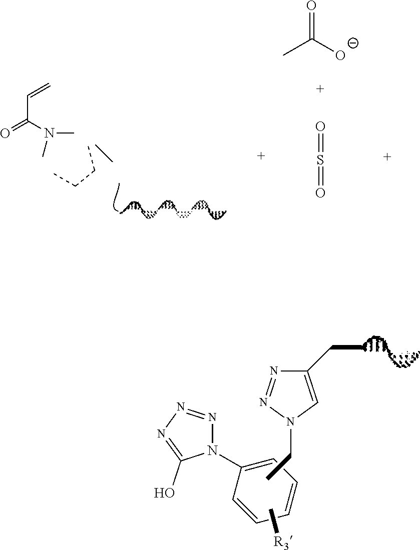

Phenyltetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 10. Synthesis of Pyrimidine-2-yl Sulfone DNA-Tagged Masked-Warhead Reagents

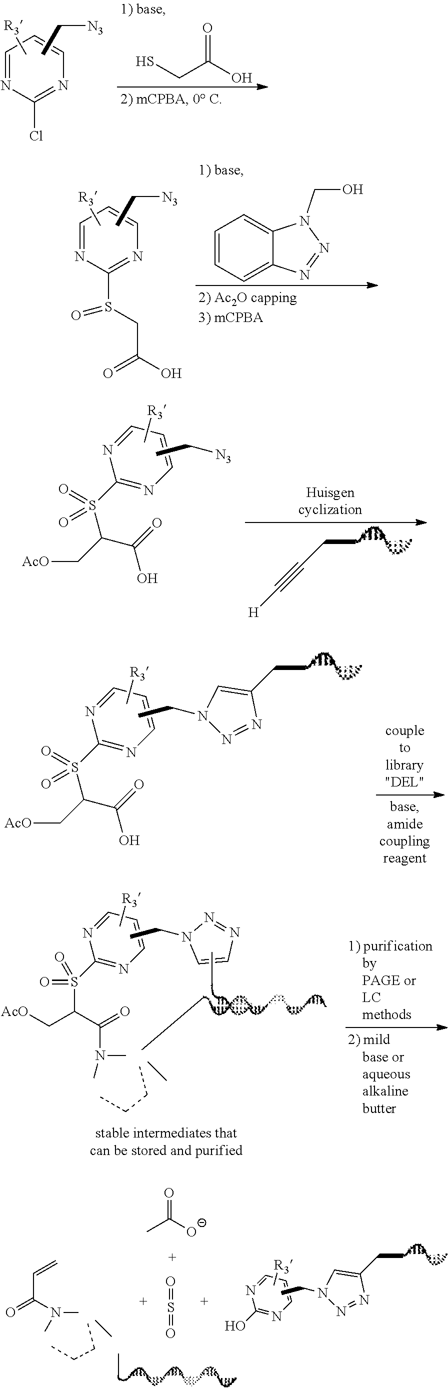

Pyrimidyl sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 11. Synthesis of Benzothiazole Sulfone DNA-Tagged Masked-Warhead Reagents

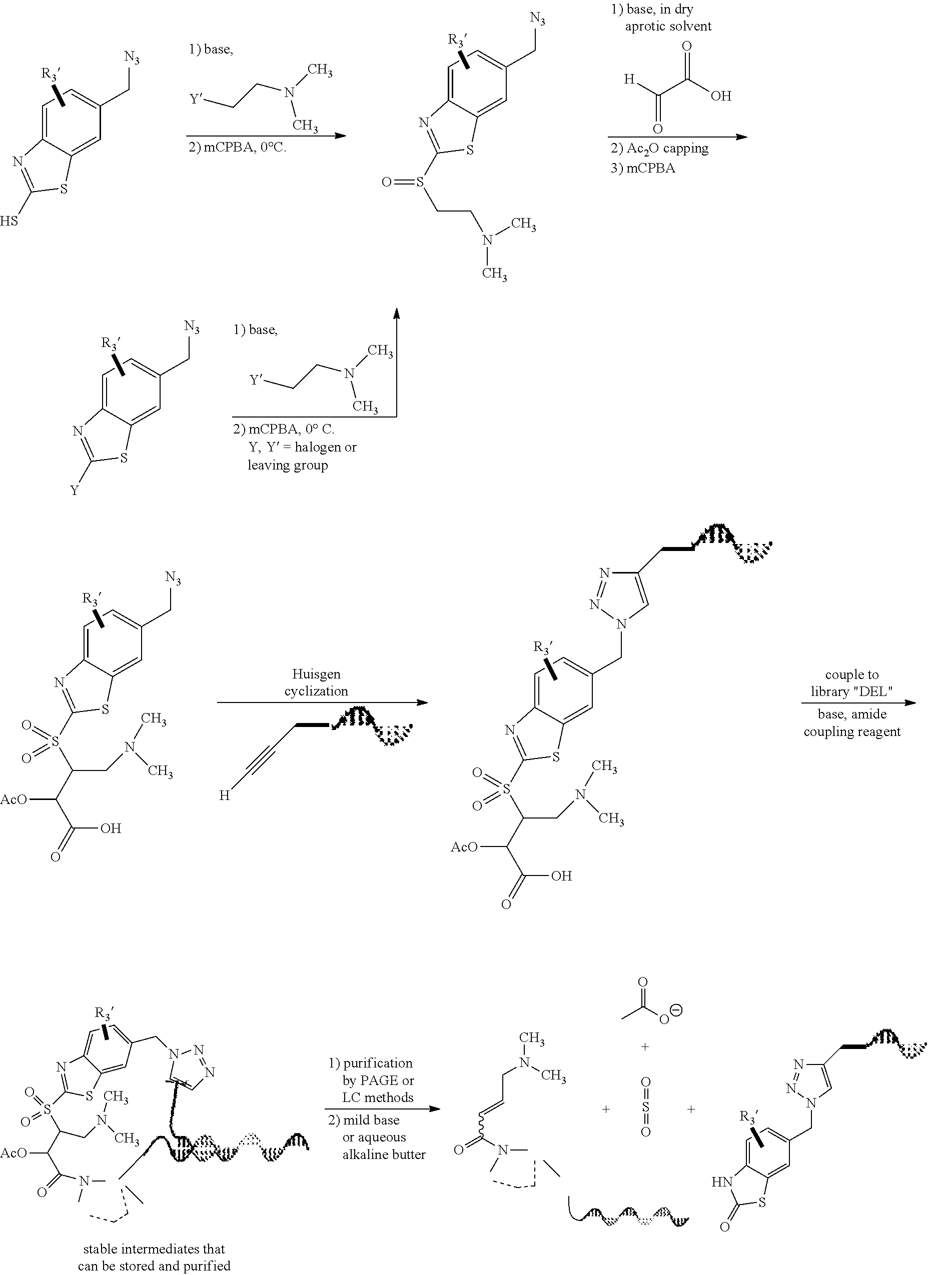

Benzothiazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 12. Synthesis of Benzothiazole Sulfone DNA-Tagged Masked-Warhead Reagents

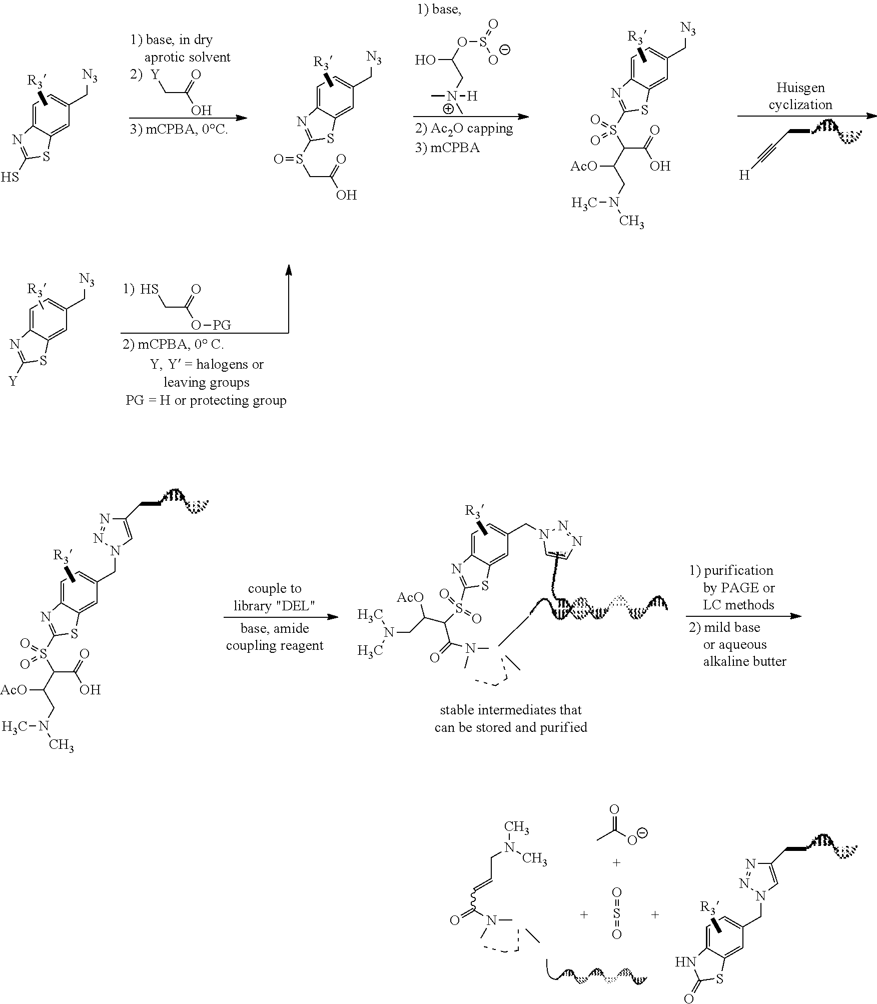

Benzothiazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 13. Synthesis of Benzothiazole Sulfone Biotin-Tagged Masked-Warhead Reagents

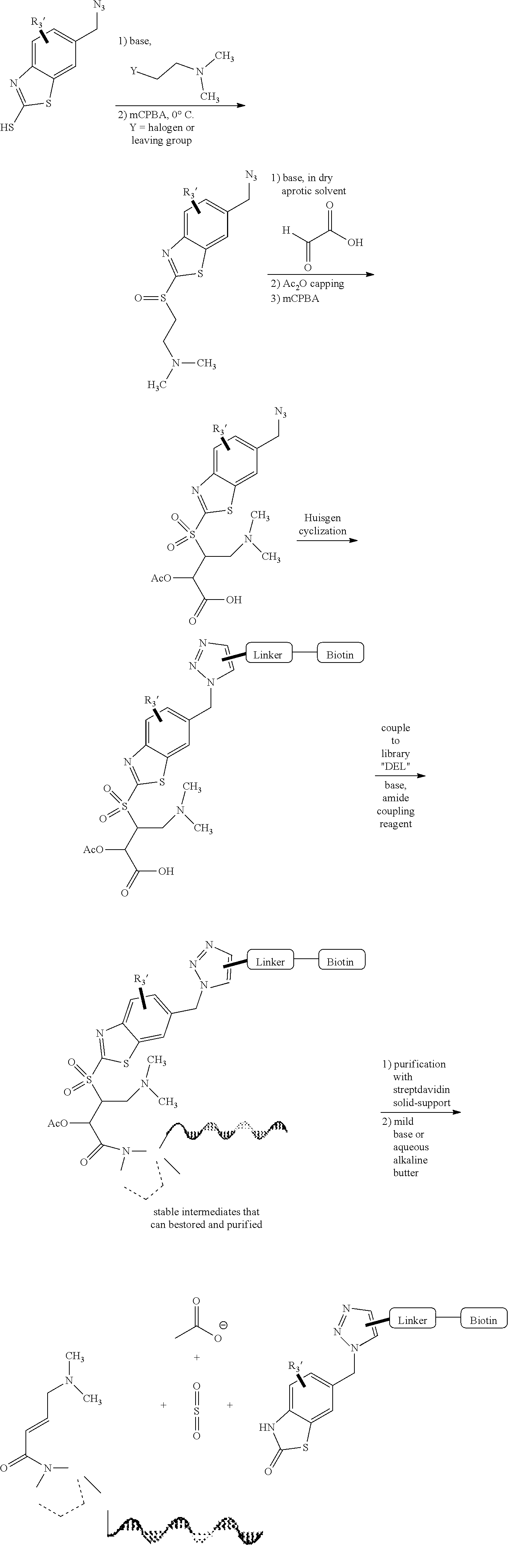

Benzothiazole sulfone biotin-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 14. Synthesis of Benzothiazole Sulfone Biotin-Tagged Masked-Warhead Reagents

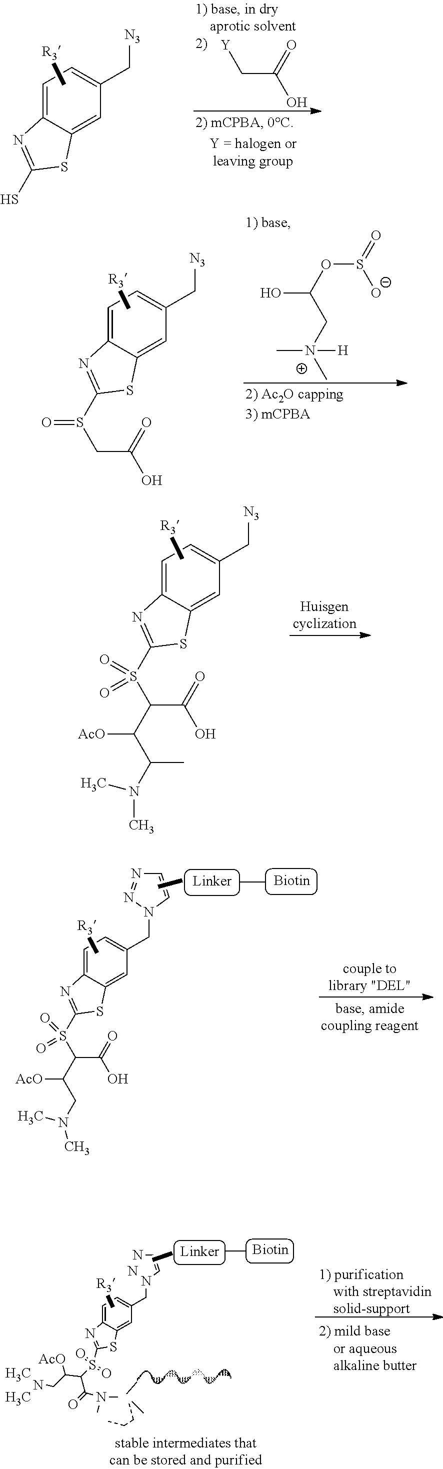

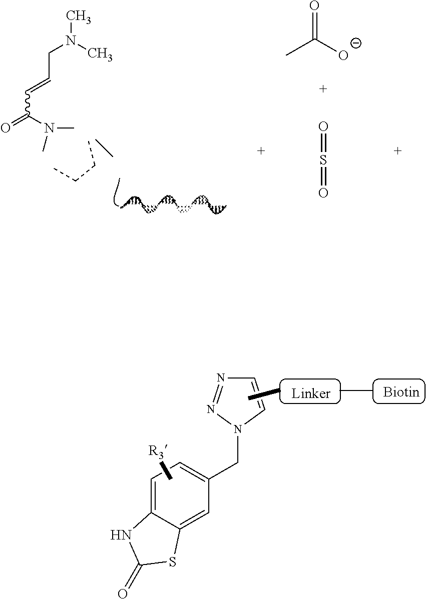

Benzothiazole sulfone biotin-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 15. Synthesis of 1-Alkyl-1H-Tetrazole-5-yl Sulfone DNA-Tagged Masked-Warhead Reagents

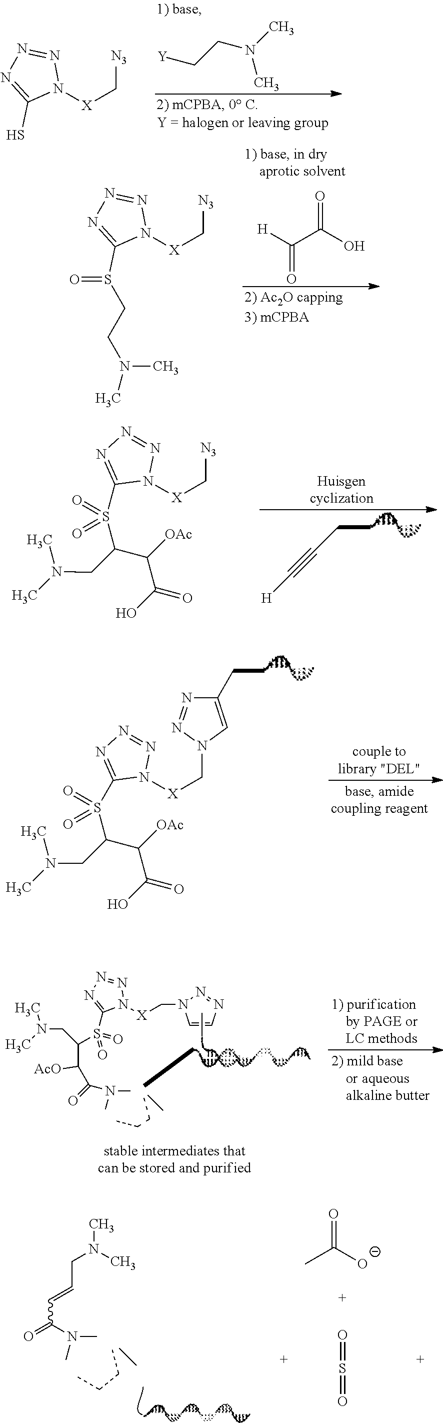

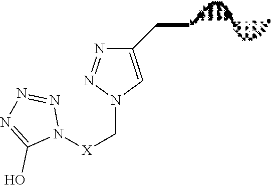

Alkytetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Kei et al., European Journal of Medicinal Chemistry, 89:207-251 (2015), Kolb et al., Angew. Chem. It. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov eta., Nat. Chem. 10(7):704-714 (2018), and International Patent Publication No. WO 2019/168654.

### Example 16. Synthesis of 1-Phenyl-1H-Tetrazole-5-yl Sulfone DNA-Tagged Masked-Warhead Reagents

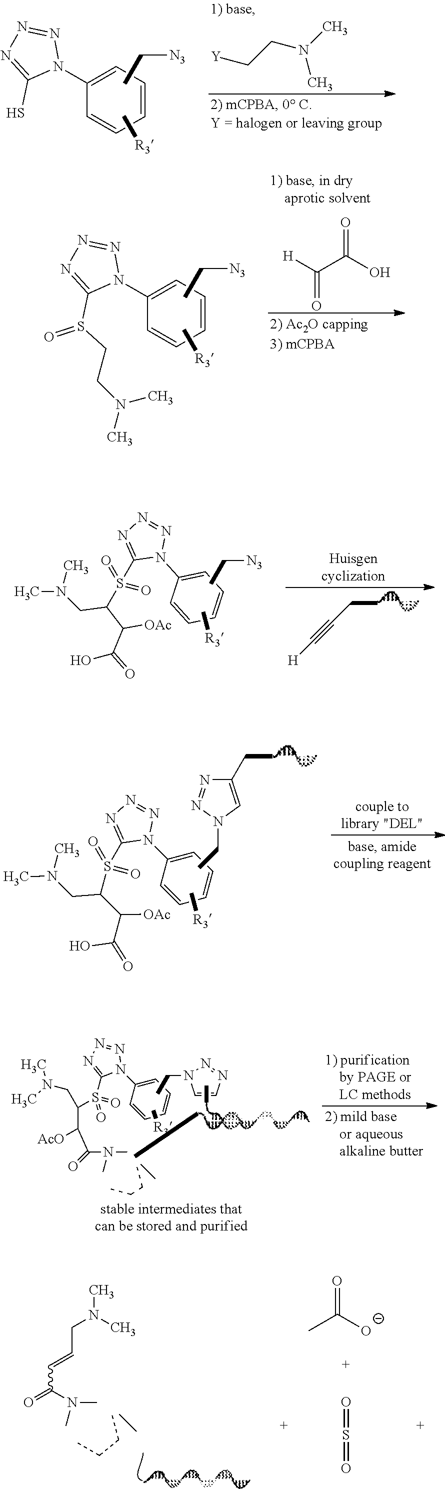

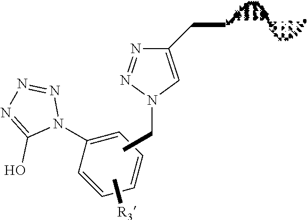

Phenyltetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 17. Synthesis of Pyrimidine-2-yl Sulfone DNA-Tagged Masked-Warhead Reagents

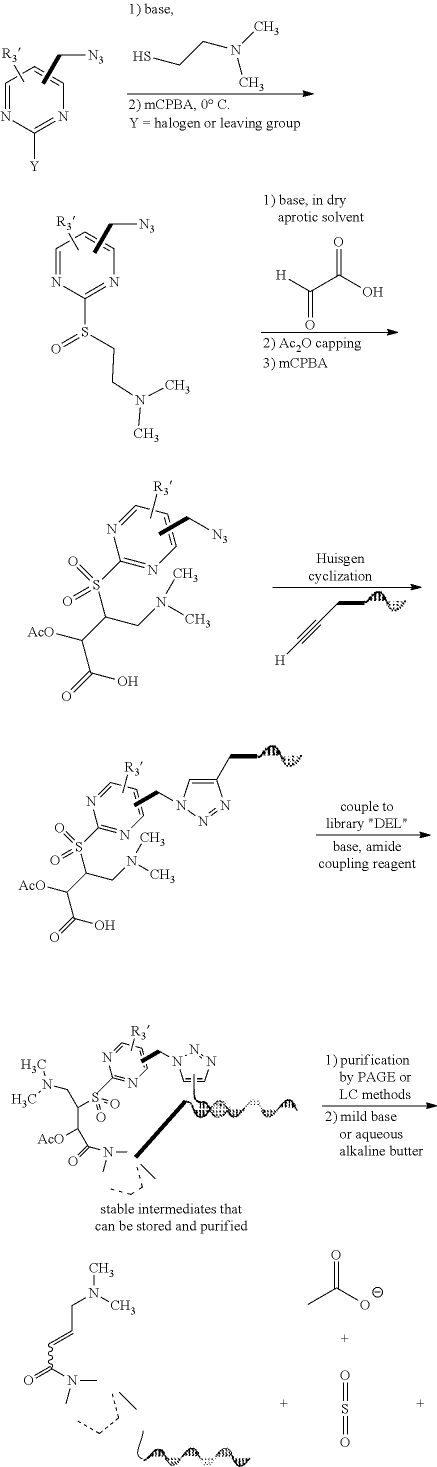

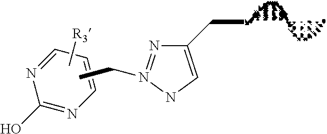

Pyrimidyl sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keni et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 18. Synthesis of 1-Alkyl-1H-Tetrazole-5-yl Sulfone DNA-Tagged Masked-Warhead Reagents

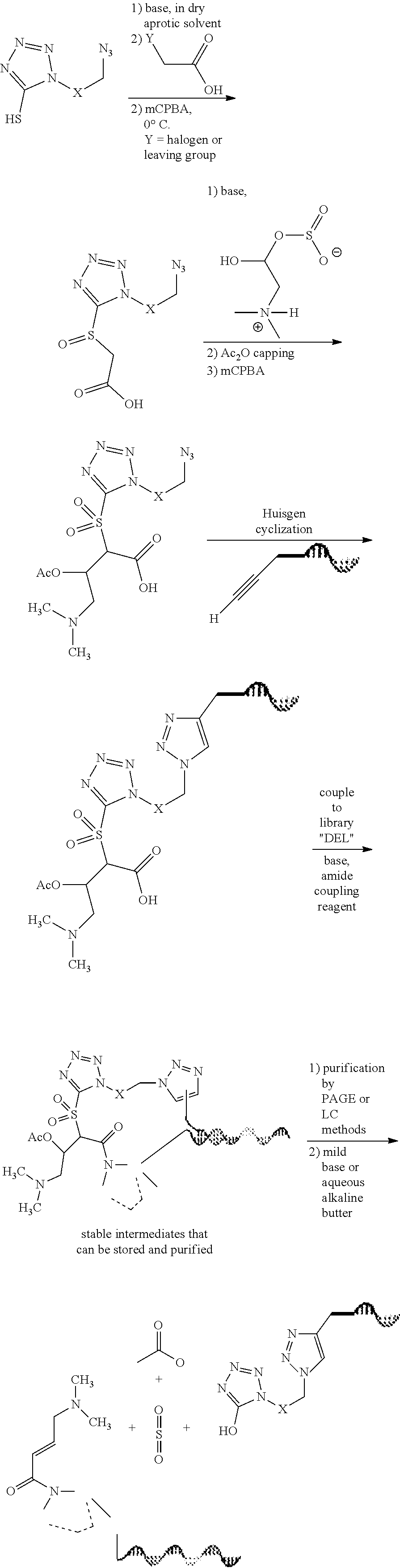

Alkytetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 19. Synthesis of 1-Phenyl-1H-Tetrazole-5-yl Sulfone DNA-Tagged Masked-Warhead Reagents

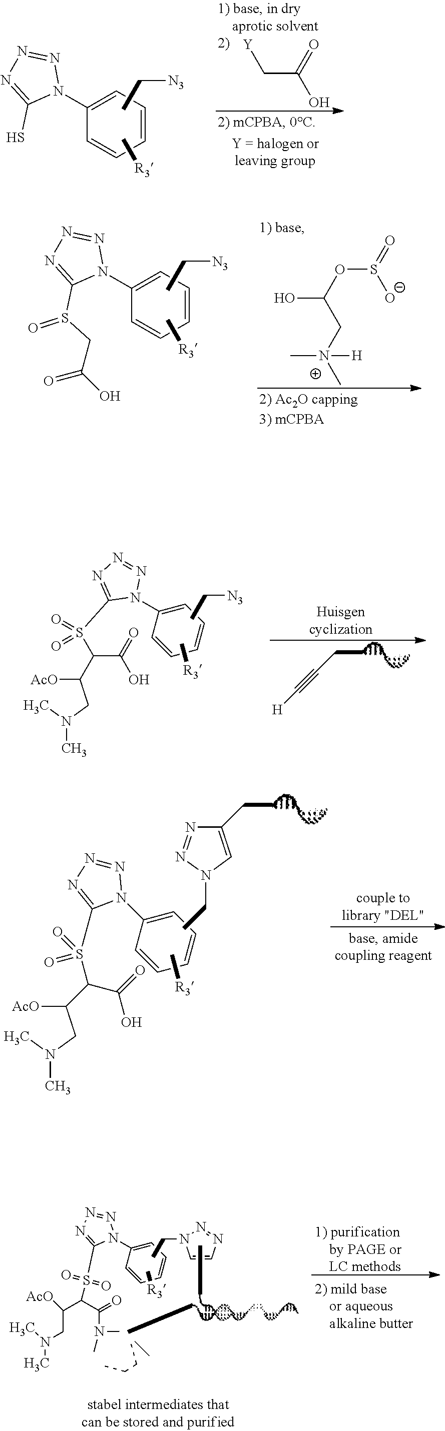

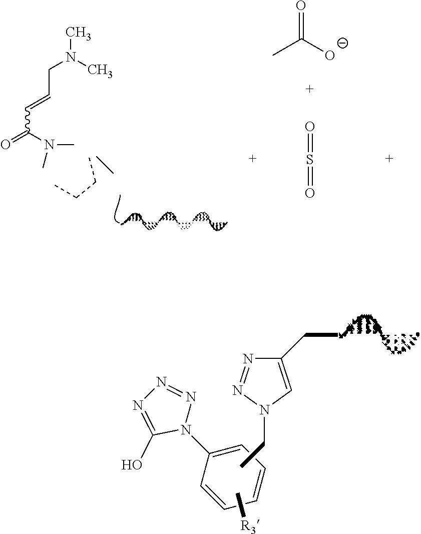

Phenyltetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 20. Synthesis of Pyrimidine-2-yl Sulfone DNA-Tagged Masked-Warhead Reagents

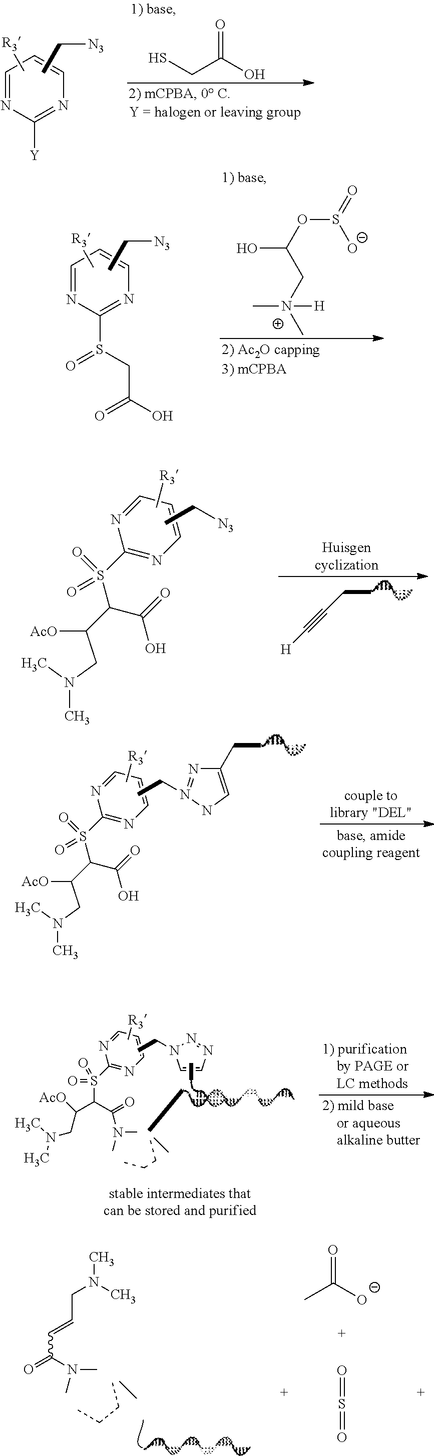

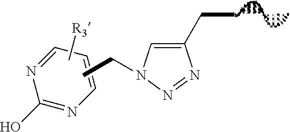

Pyrimidyl sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 21. Synthesis of Benzothiazole Sulfone DNA-Tagged Masked-Warhead Reagents

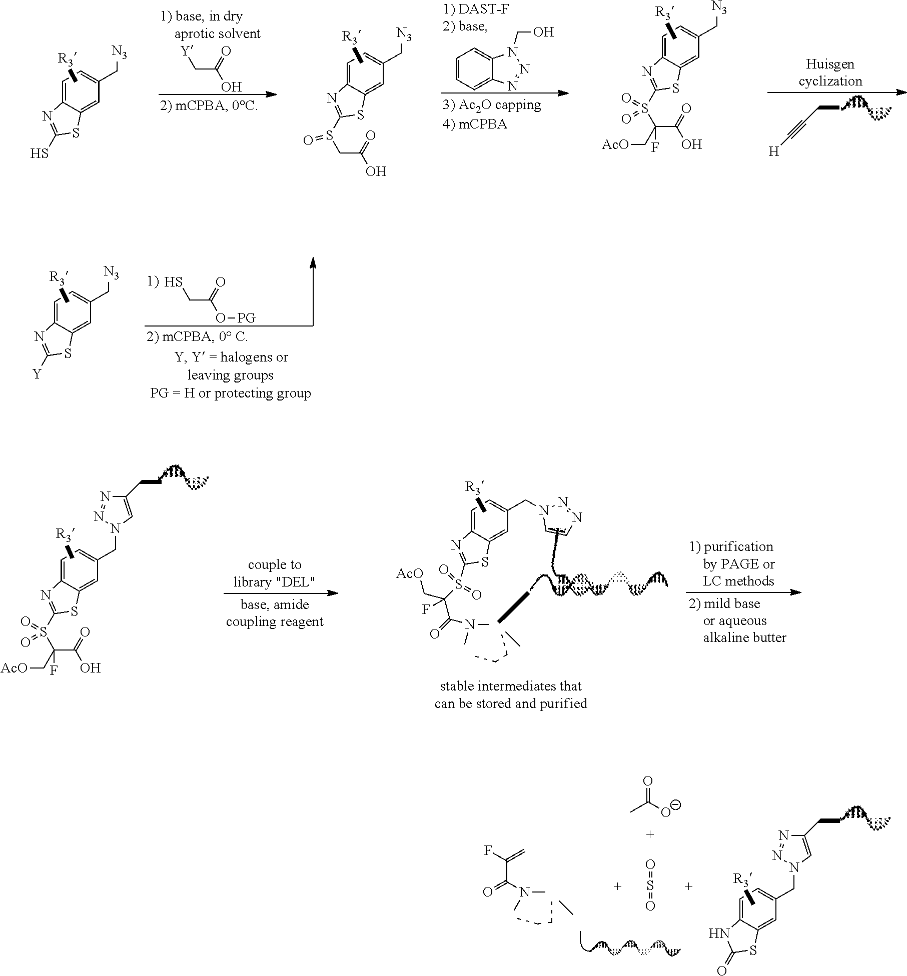

Benzothiazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 22. Synthesis of Benzothiazole Sulfone Biotin-Tagged Masked-Warhead Reagents

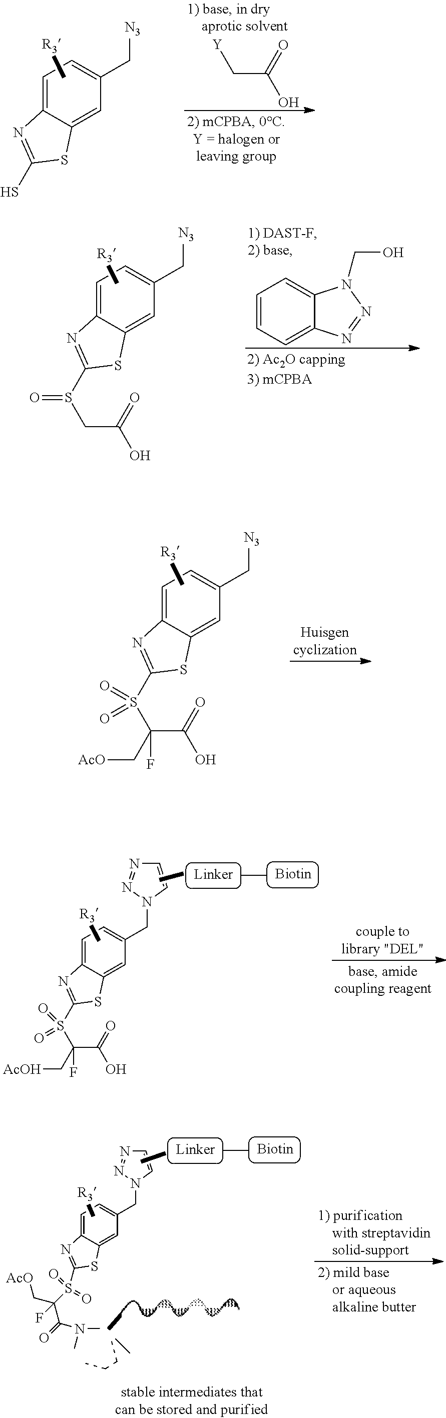

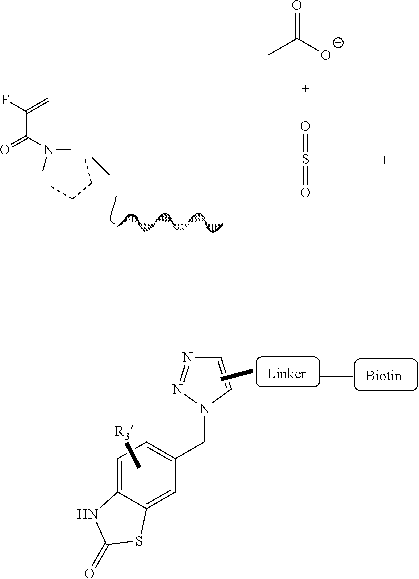

Benzothiazole sulfone biotin-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 23. Synthesis of 1-Alkyl-1H-Tetrazole-5-yl Sulfone DNA-Tagged Masked-Warhead Reagents

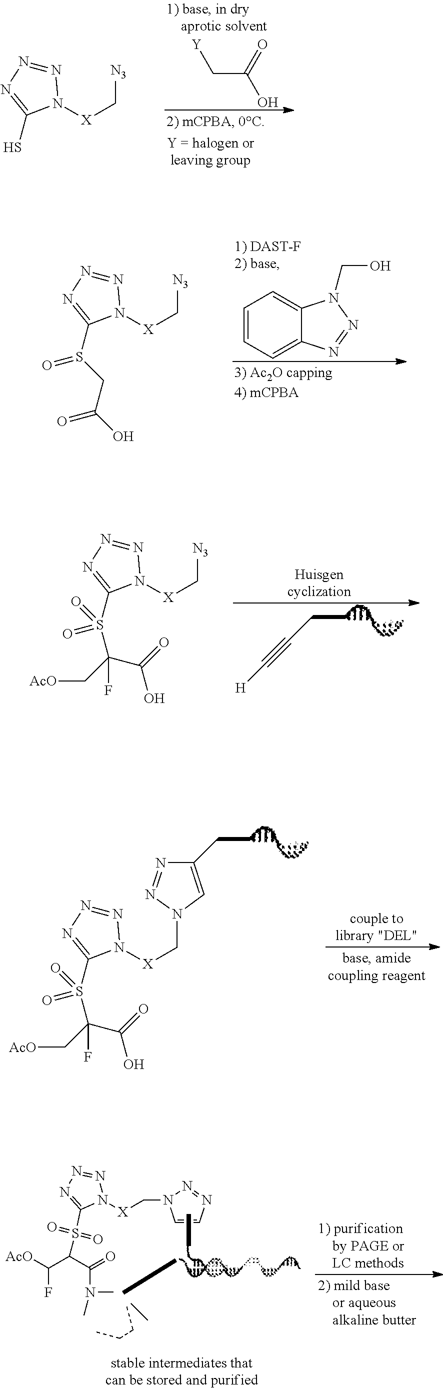

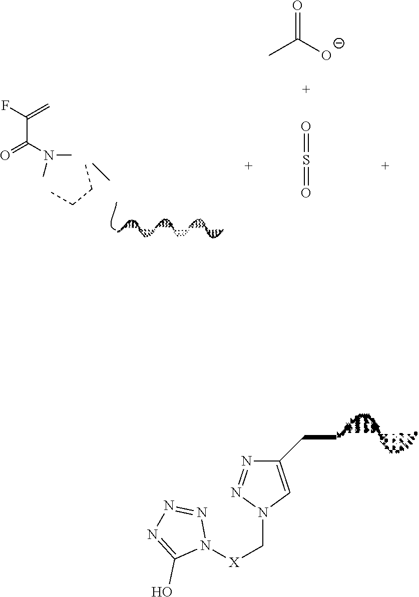

Alkytetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 24. Synthesis of 1-Phenyl-1H-Tetrazole-5-yl Sulfone DNA-Tagged Masked-Warhead Reagents

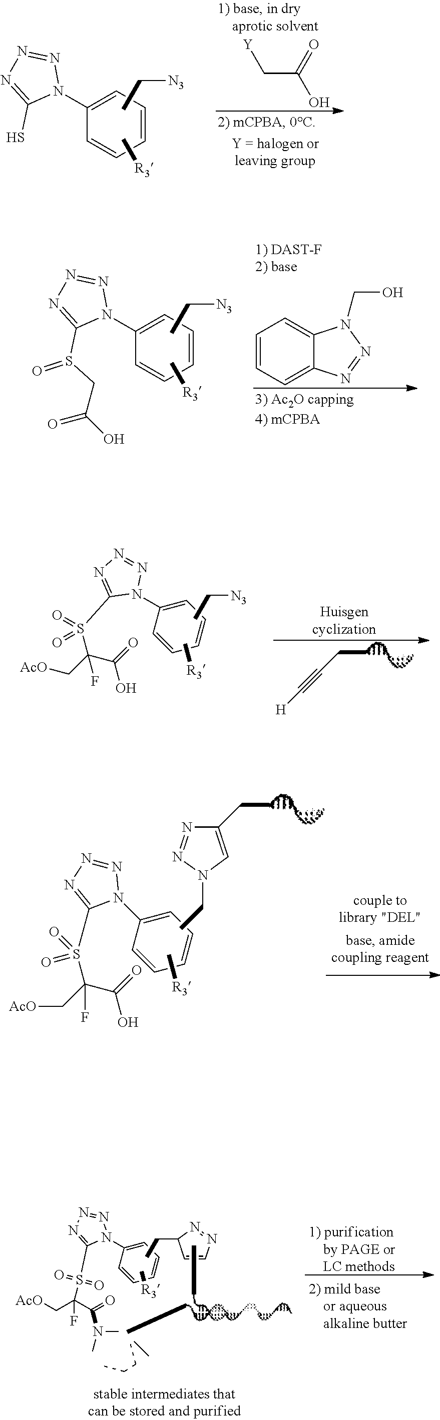

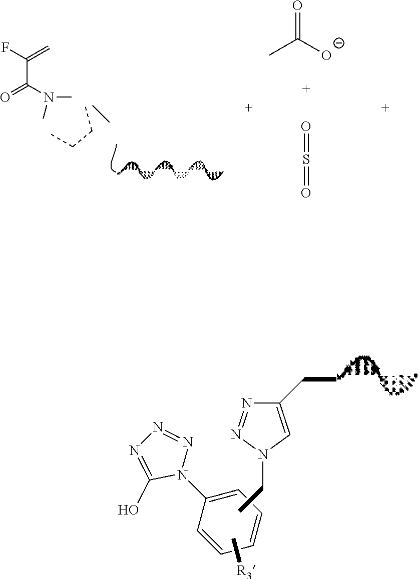

Phenyltetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 25. Synthesis of Pyrimidine-2-yl Sulfone DNA-Tagged Masked-Warhead Reagents

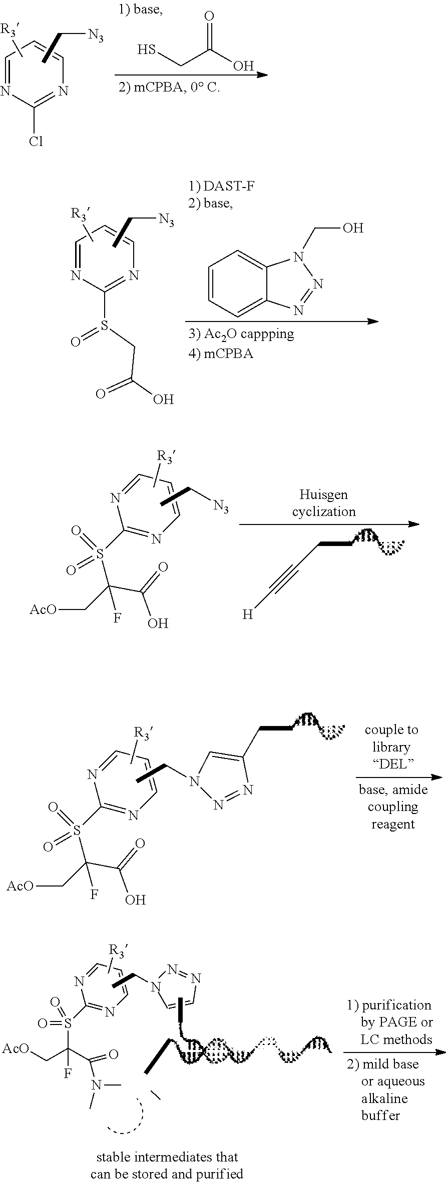

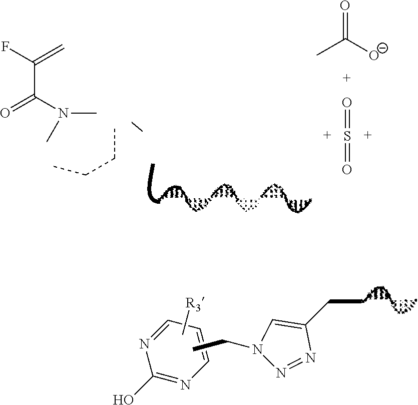

Pyrimidyl sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 26. Synthesis of Benzothiazole Sulfone DNA-Tagged Masked-Warhead Reagents

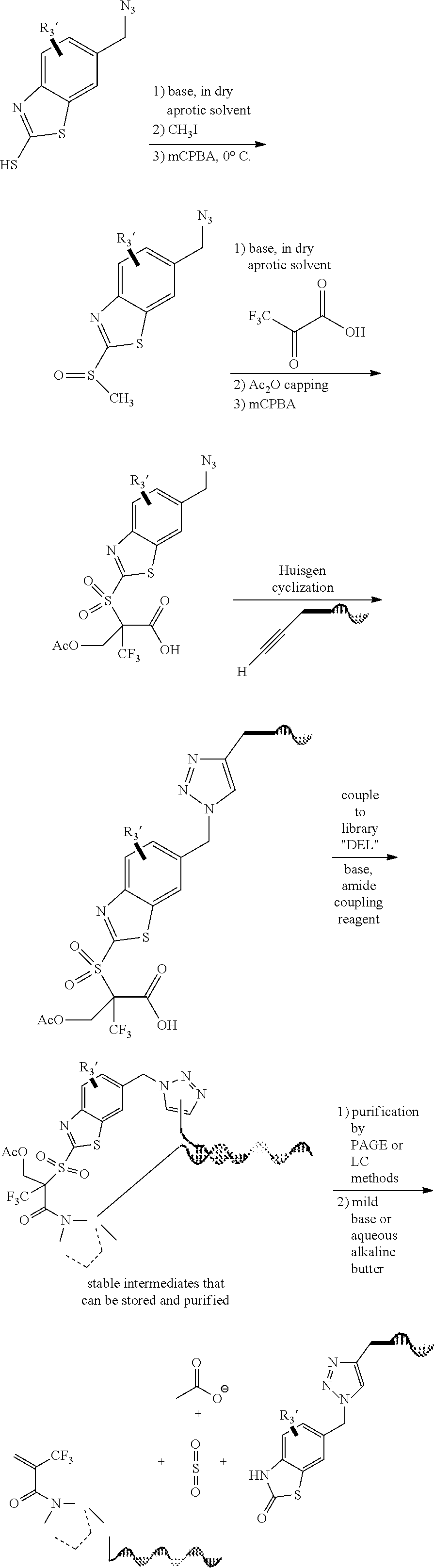

Benzothiazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 27. Synthesis of Benzothiazole Sulfone Biotin-Tagged Masked-Warhead Reagents

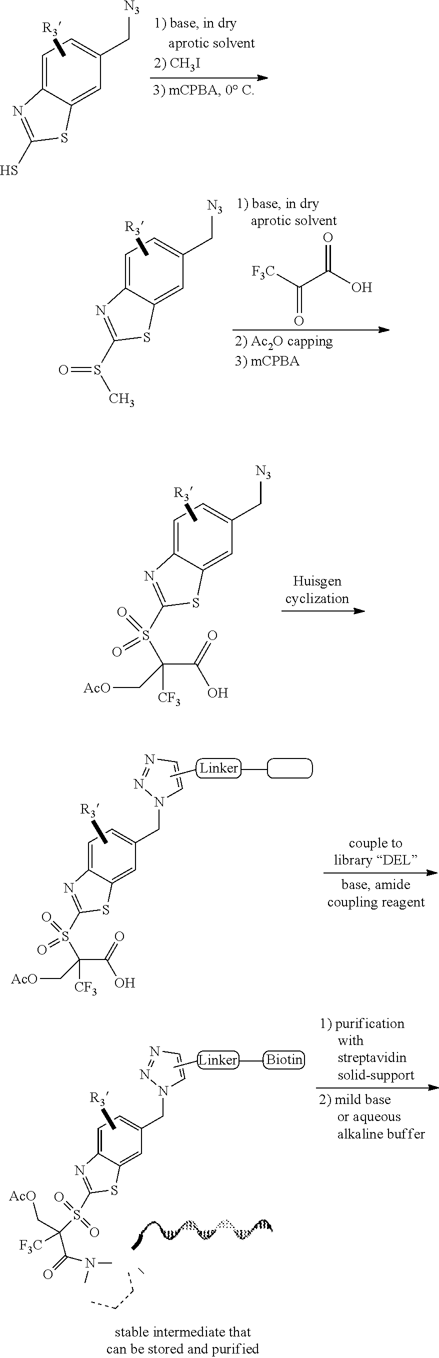

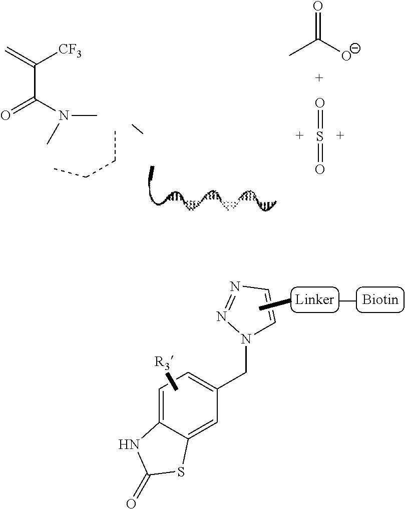

Benzothiazole sulfone biotin-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 28. Synthesis of 1-Alkyl-1H-Tetrazole-5-yl Sulfone DNA-Tagged Masked-Warhead Reagents

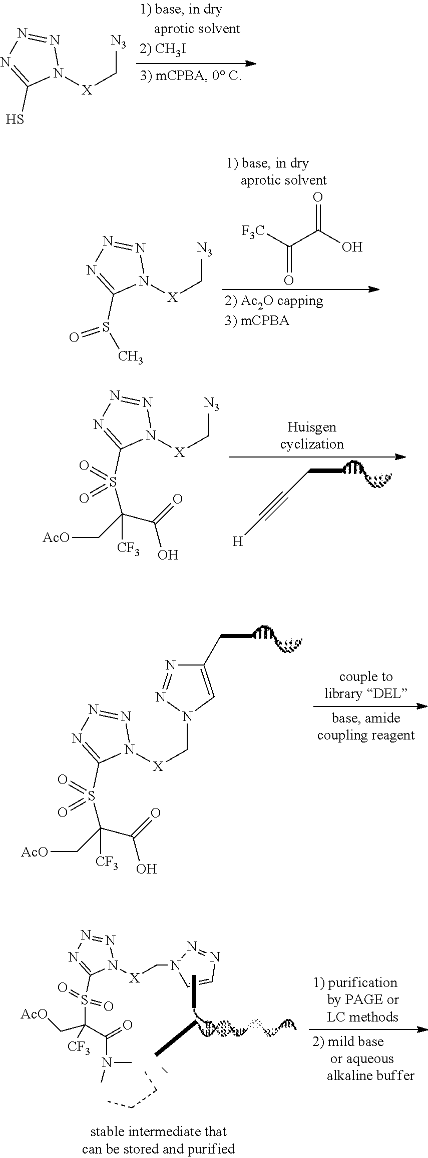

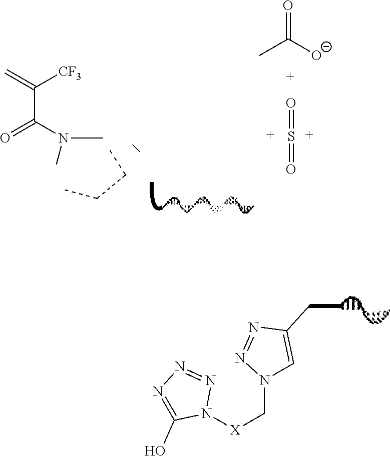

Alkytetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 29. Synthesis of 1-Phenyl-1H-Tetrazole-5-yl Sulfone DNA-Tagged Masked-Warhead Reagents

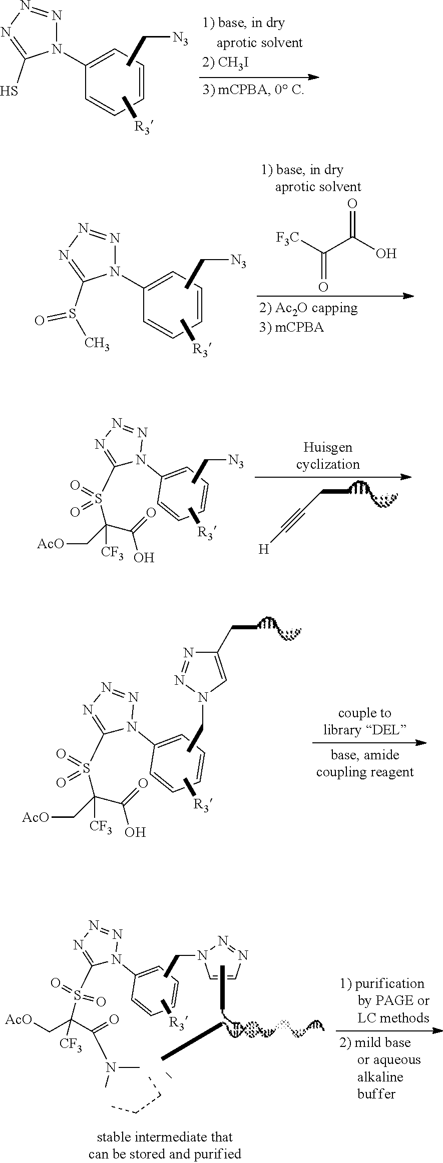

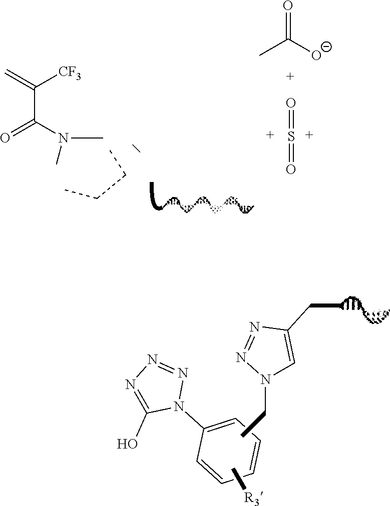

Phenyltetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 30. Synthesis of Pyrimidine-2-yl Sulfone DNA-Tagged Masked-Warhead Reagents

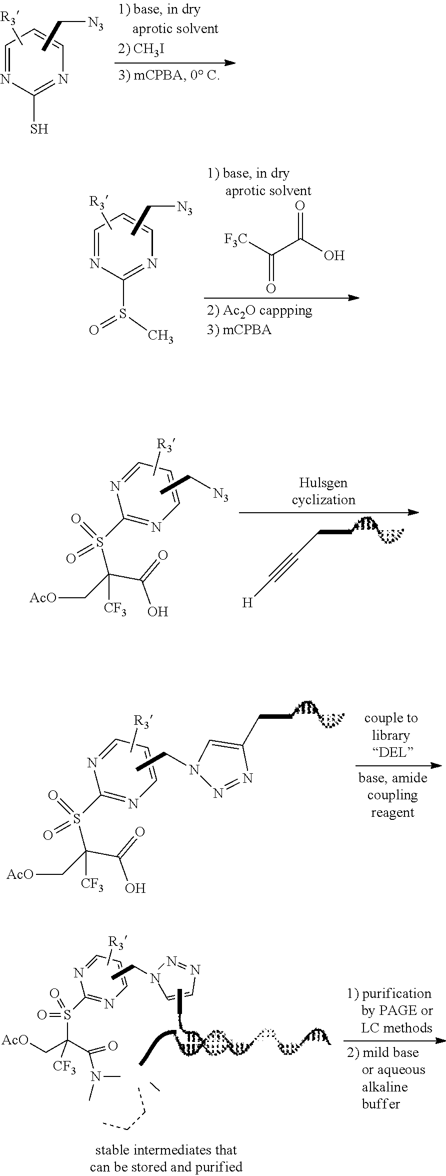

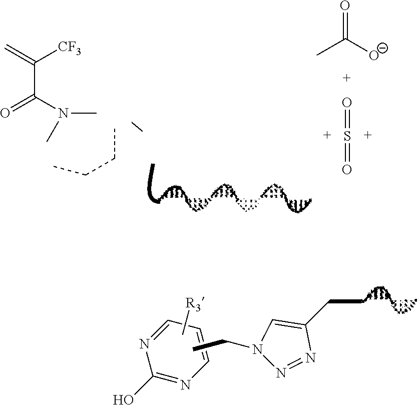

Pyrimidyl sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 31. Synthesis of Benzothiazole Sulfone DNA-Tagged Masked-Warhead Reagents

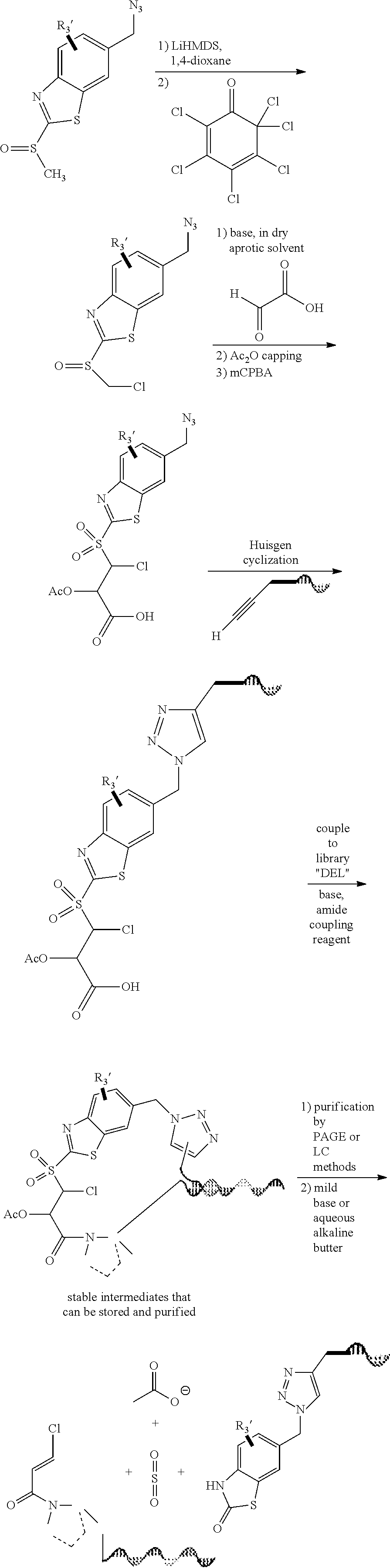

Benzothiazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 32. Synthesis of Benzothiazole Sulfone Biotin-Tagged Masked-Warhead Reagents

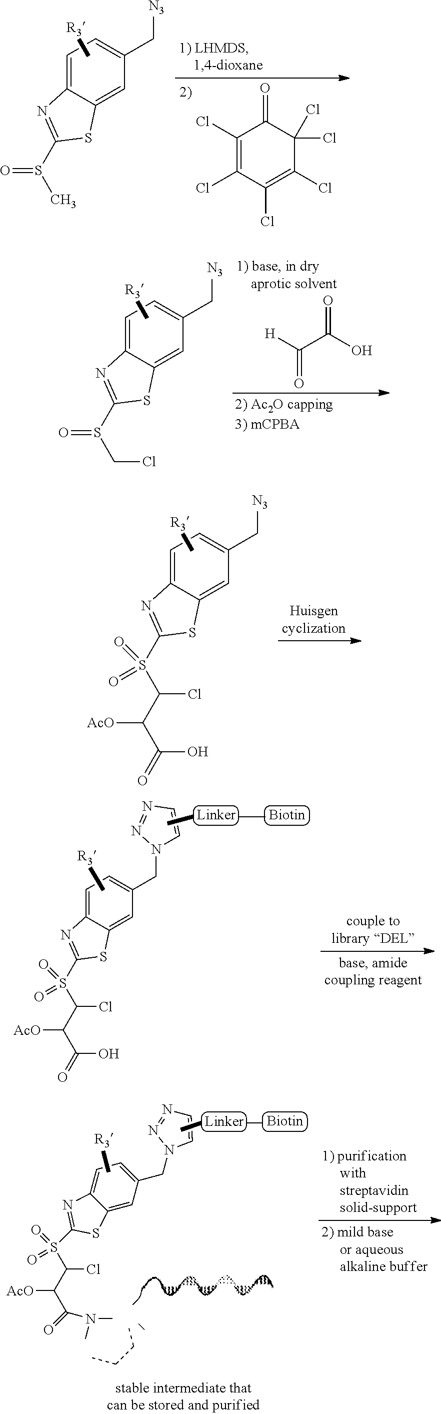

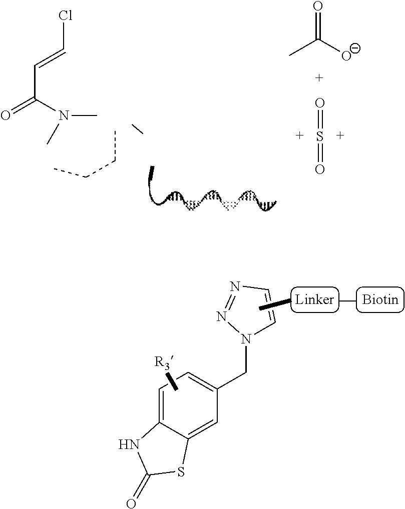

Benzothiazole sulfone biotin-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 33. Synthesis of 1-Alkyl-1H-Tetrazole-5-yl Sulfone DNA-Tagged Masked-Warhead Reagents

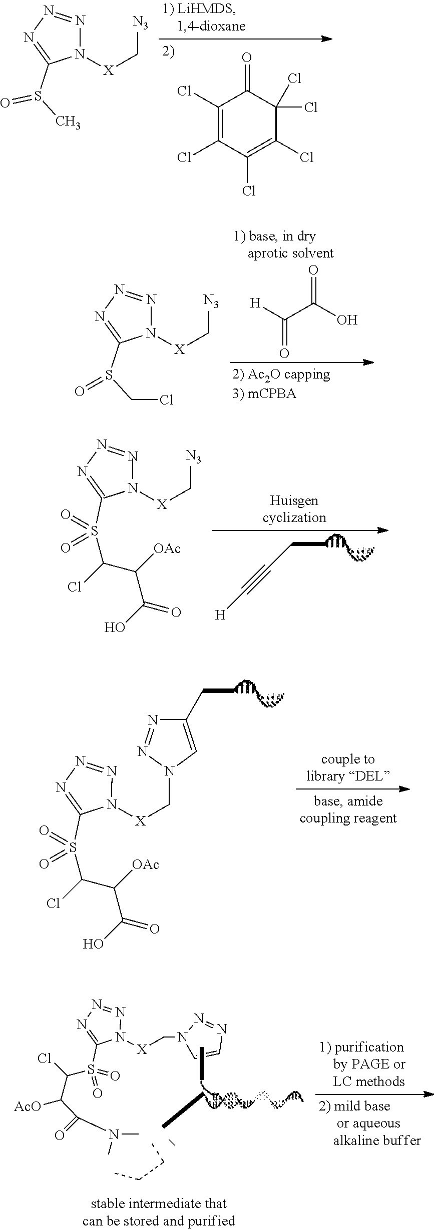

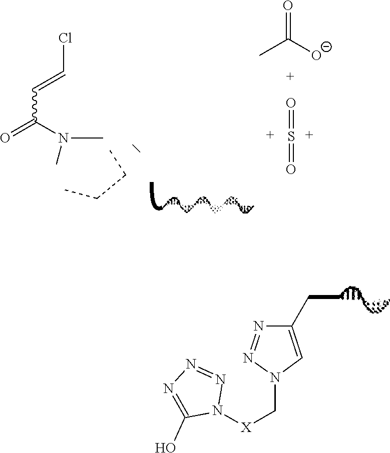

Alkytetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 34. Synthesis of 1-Phenyl-1H-Tetrazole-5-yl Sulfone DNA-Tagged Masked-Warhead Reagents

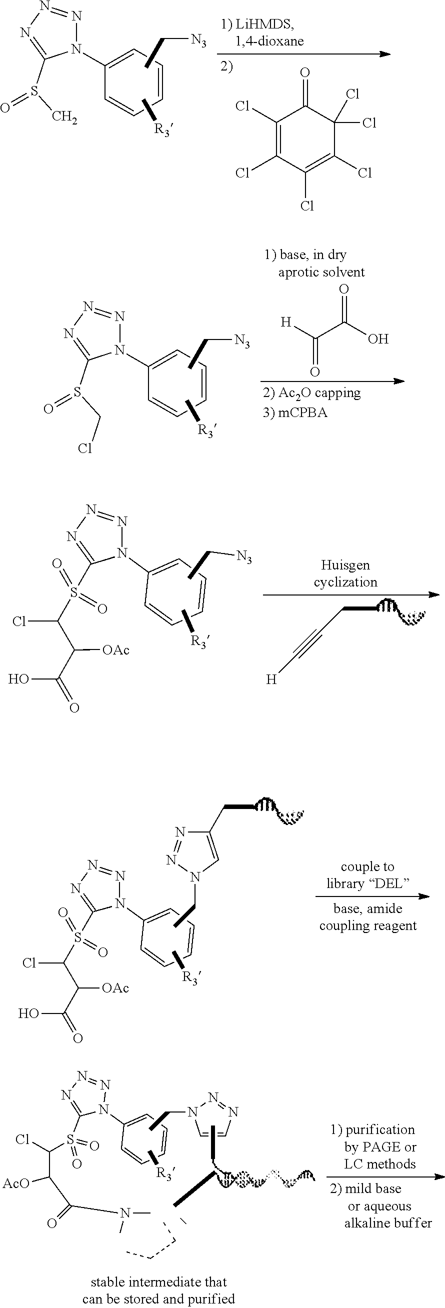

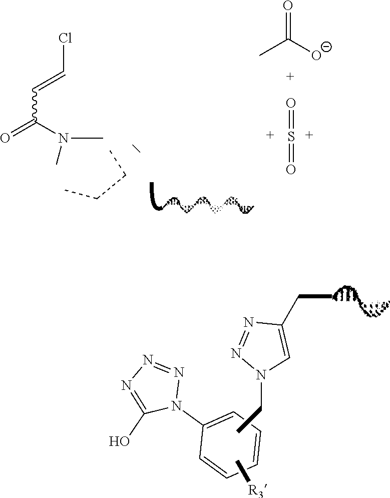

Phenyltetrazole sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 35. Synthesis of Pyrimidine-2-yl Sulfone DNA-Tagged Masked-Warhead Reagents

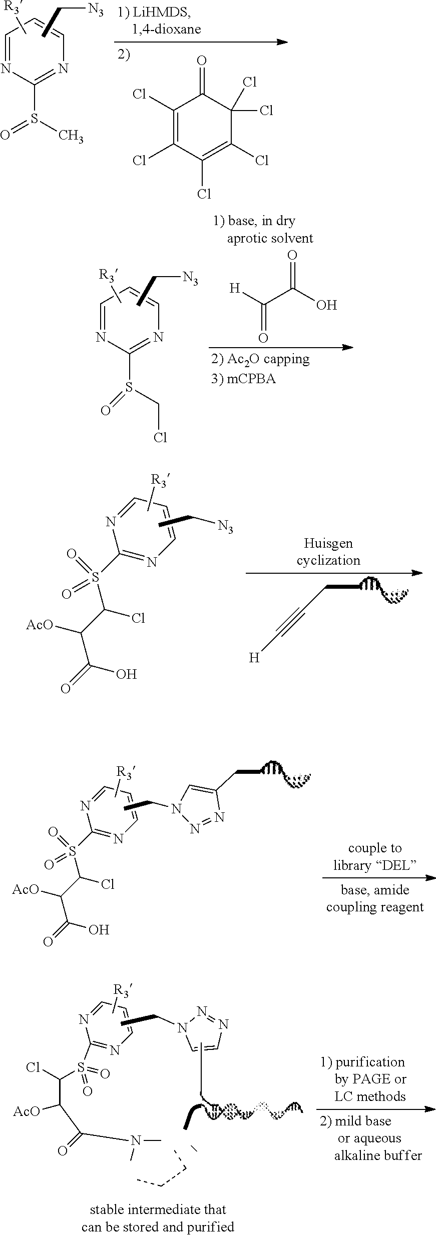

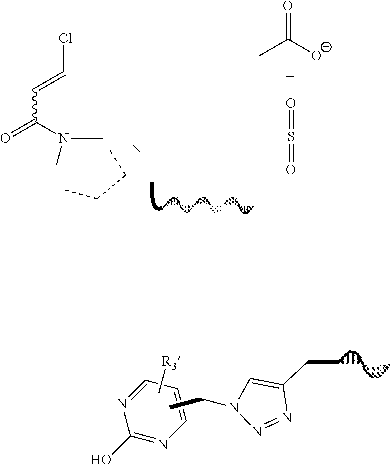

Pyrimidyl sulfone DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 36. Synthesis of Arylsilyl DNA-Tagged Masked-Warhead Reagents

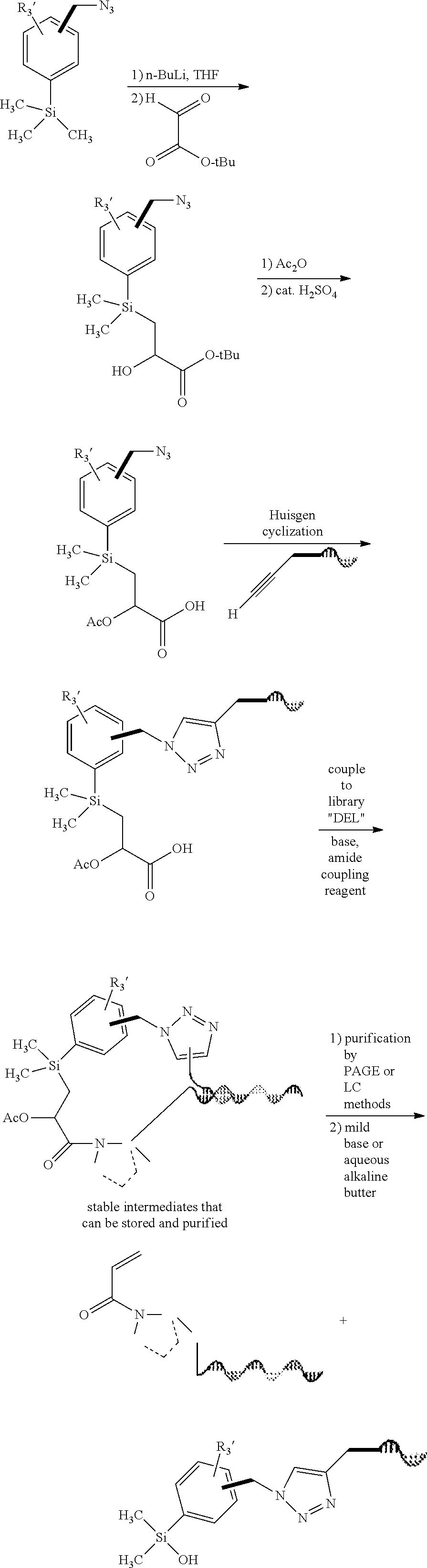

Arylsilyl DNA-tagged masked-warhead reagents are synthesized as illustrated in the above scheme. The reactions are performed as described in Bishop et al., J. Org. Chem. 56(17):5079-5091 (1991), Ager, D. J. Synthesis; 1984(5): 384-398 (1984), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 37. Synthesis of Arylsilyl Biotin-Tagged Masked-Warhead Reagents

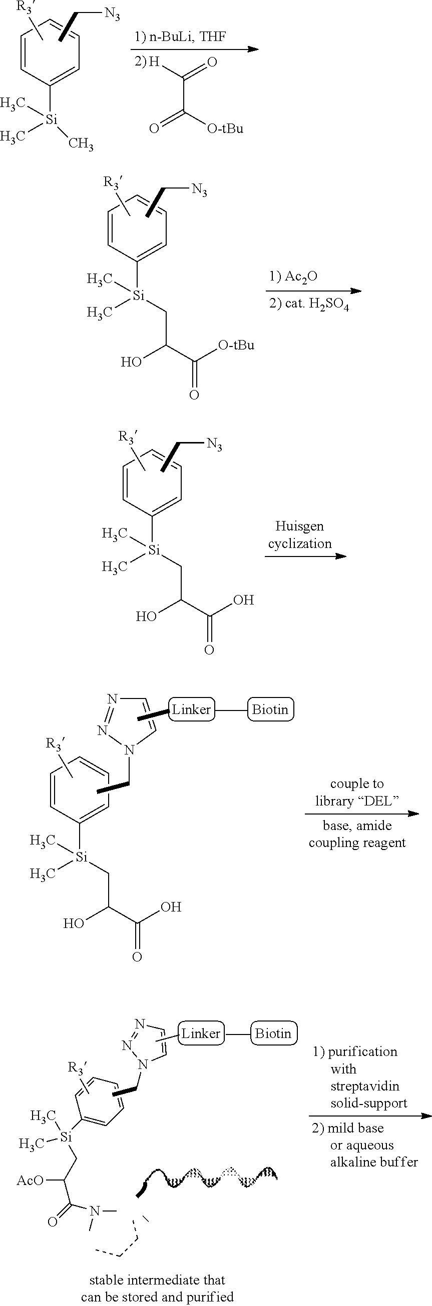

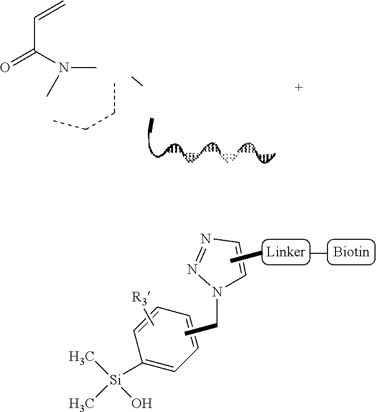

Arylsilyl DNA-tagged masked-warhead reagents are synthesized as illustrated in the above scheme. The reactions are performed as described in Bishop et al., J. Org. Chem. 56(17):5079-5091 (1991), Ager, D. J. Synthesis; 1984(5):384-398 (1984), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 38. Synthesis of Benzothiazole Sulfone DNA-Tagged Masked-Warhead Reagents Using a 3-Bromo-Pyruvate Ester and an Arylthiol Synthetic Intermediates

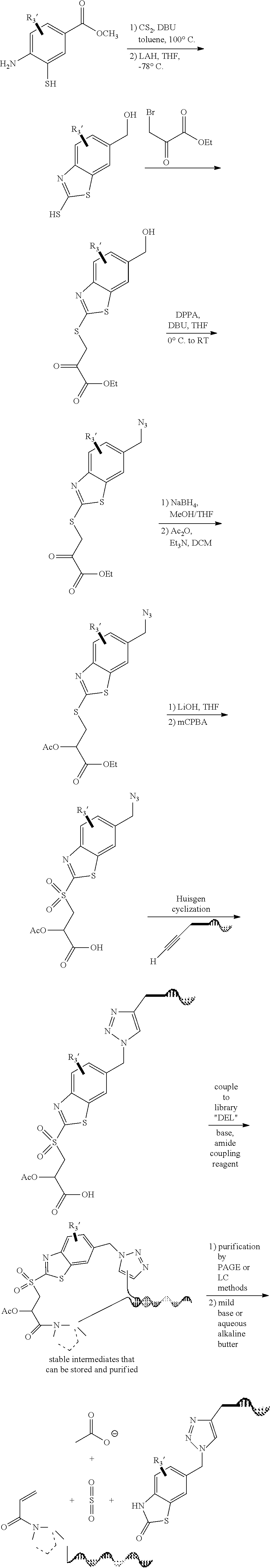

Arylsilyl DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 39. Synthesis of Benzothiazole Sulfone DNA-Tagged Masked-Warhead Reagents, Wherein a Ketone Reduction Step Mediates the Release of the Acrylamide eDEL

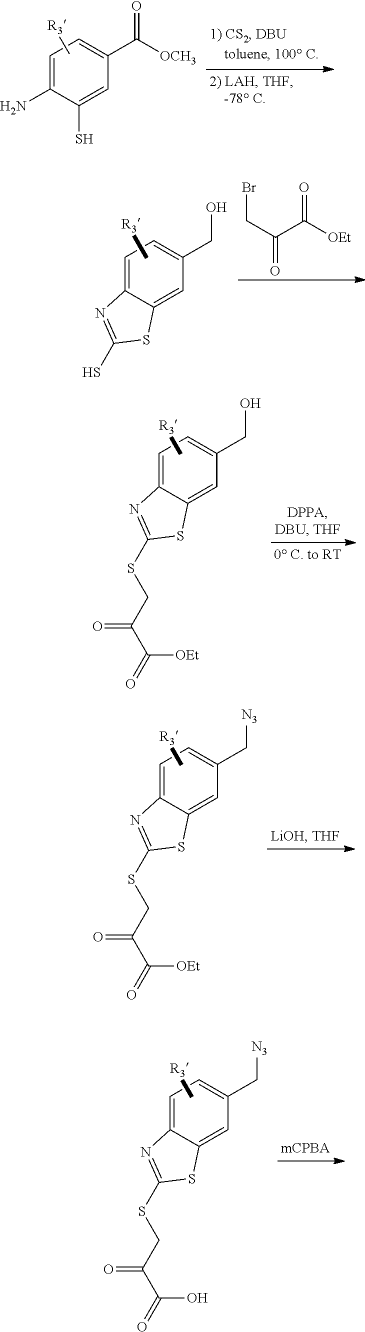

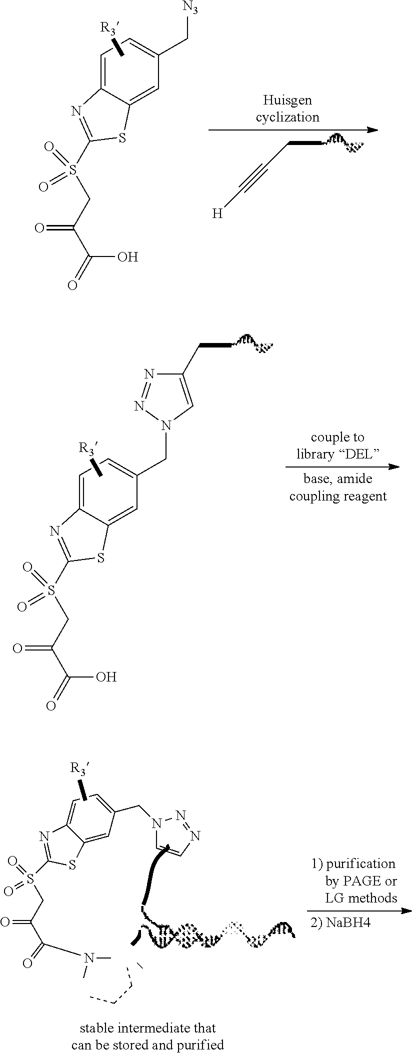

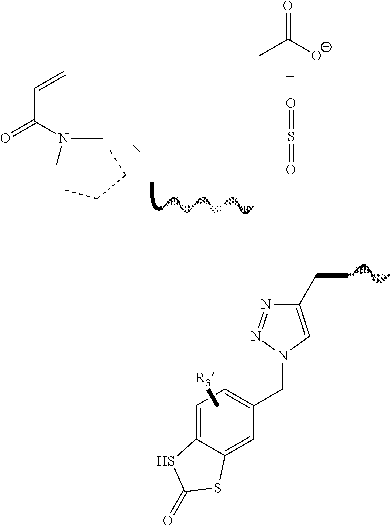

Arylsilyl DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. It. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 40. Synthesis of Benzothiazole Sulfone DNA-Tagged Masked-Warhead Reagents Using an Epoxide and Arylthiol Synthetic Intermediates

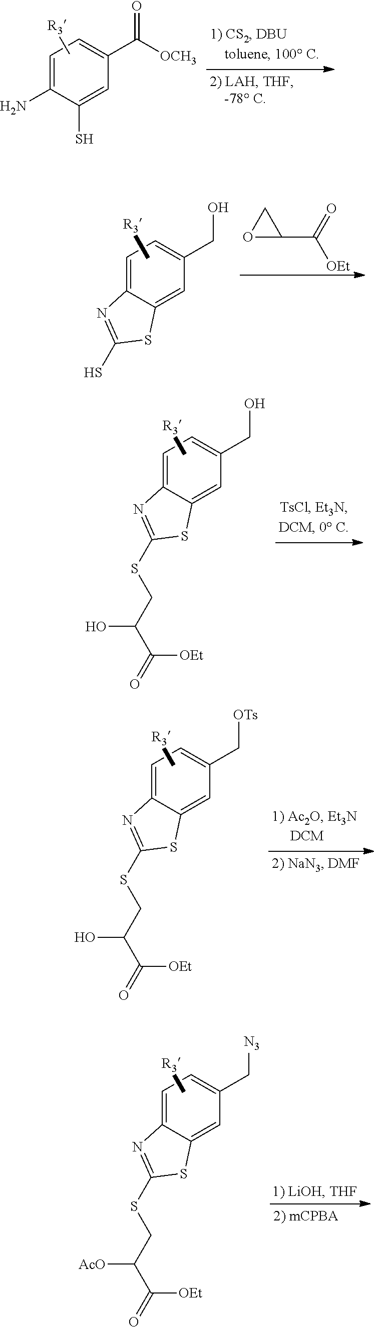

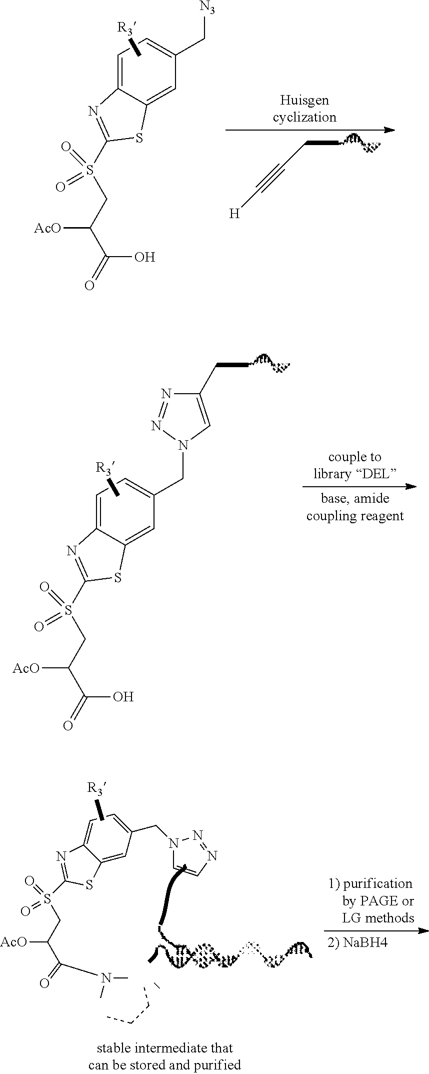

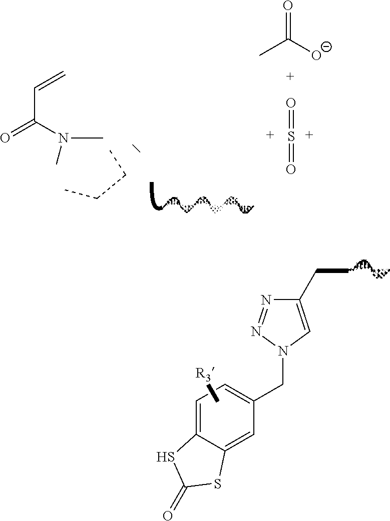

Arylsilyl DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Ken eta., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 41. Synthesis of Benzothiazole Sulfone DNA-Tagged Masked-Warhead Reagents Using a 2-Chlorobenzothiazole Intermediate and 2-Acetoxy-3-Mercaptopropanoic Acid

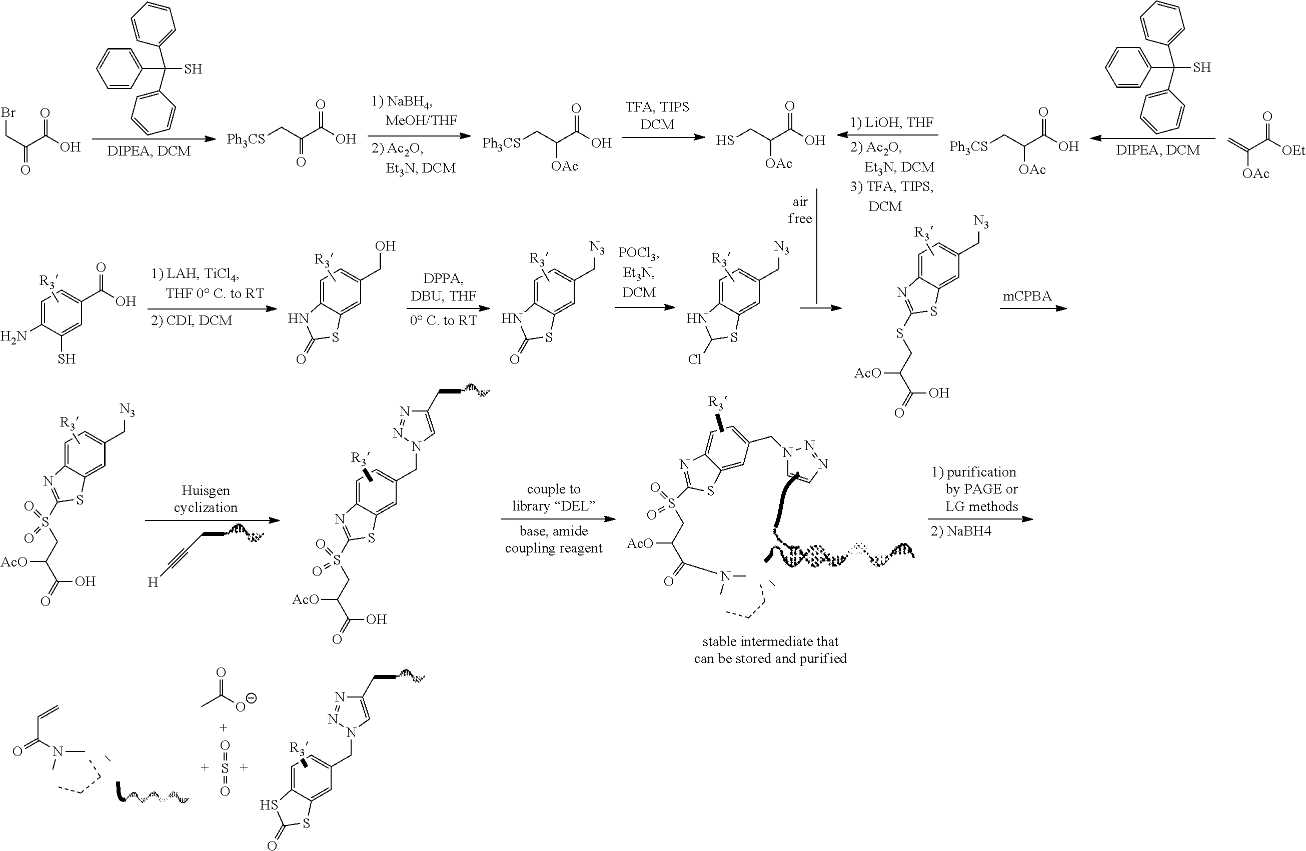

Arylsilyl DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Galardon et al., ChemBioChem, 19(16) 1702-1705 (2018), Reddy et al., Org. Lett. 2019, 21, 24, 9965-9969, Batt-Coutrot et al., Macromol. Chem. Phys., 206, 1709-1717 (2005), Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 42. Synthesis of Benzothiazole Sulfone DNA-Tagged Masked-Warhead Reagents by Acylation of a Click Chemistry Handle onto a Mercaptobenzothiazole Intermediate

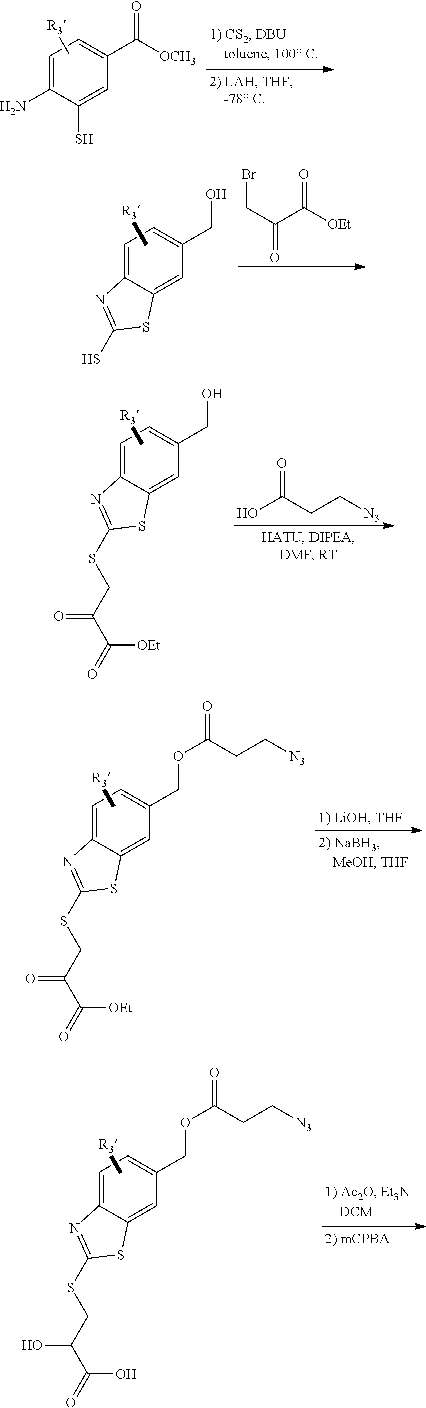

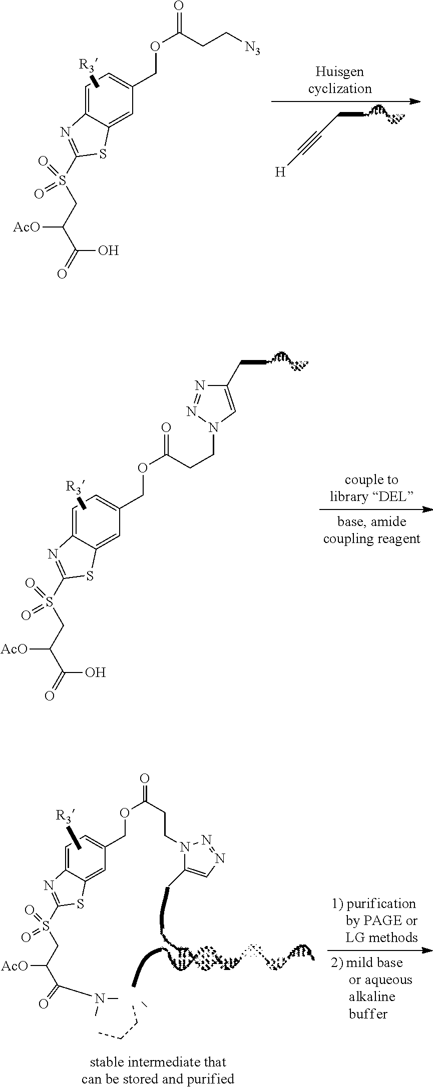

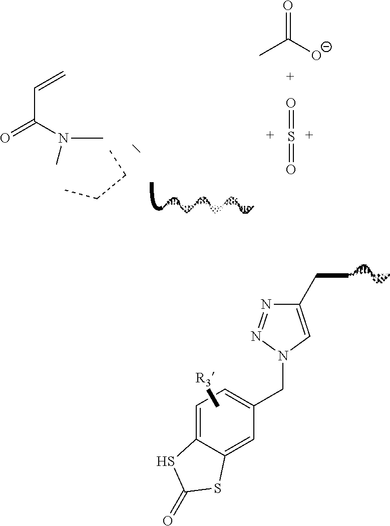

Arylsilyl DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 43. Synthesis of Benzothiazole Sulfone Biotin-Tagged Masked-Warhead Reagents by Acylation of a Biotinylated Linker onto a Mercaptobenzothiazole Intermediate

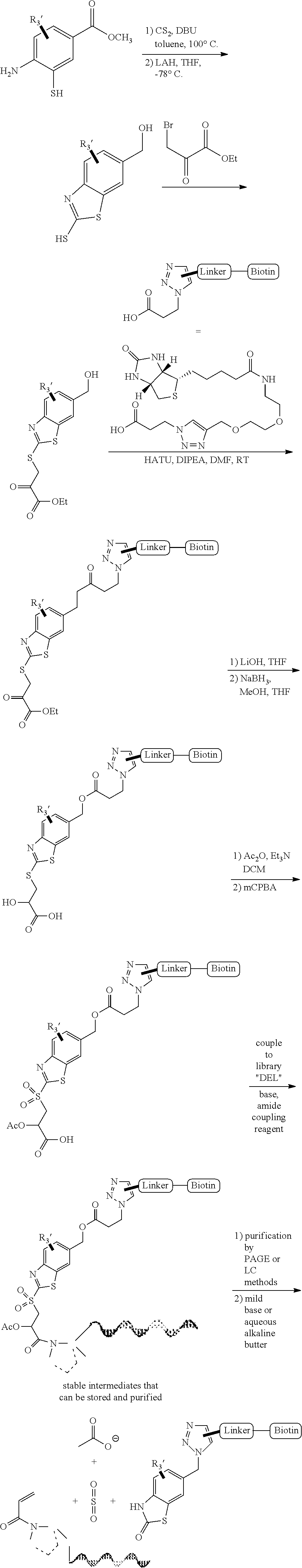

Arylsilyl DNA-tagged masked-warhead reagents are synthesized as illustrated in the above scheme. The reactions are performed as described in Blakemore et al., The Julia-Kocienski Olefination, Organic Reactions, John Wiley & Sons, Inc.: Hoboken, N.J., USA, 2018, pp 1-261, Keri et al., European Journal of Medicinal Chemistry, 89:207-251 (2015), Kolb et al., Angew. Chem. Int. Ed., 40:2004-2021 (2001), Fantoni et al., Chem. Rev. 121(12):7122-7154 (2021), Gartner et al., Science 305:1601-1605 (2004), Tse et al., J. Am. Chem. Soc. 130:15611-15626 (2008), Usanov et al., Nat. Chem. 10(7):704-714 (2018), and International Patent Publication No. WO 2019/168654.

### Example 44. Synthesis of an Analog of the Covalent EGFR Inhibitor “Afatinib” Using a Biotin-Tagged Masked Warhead Reagent to Install the Acrylamide Group

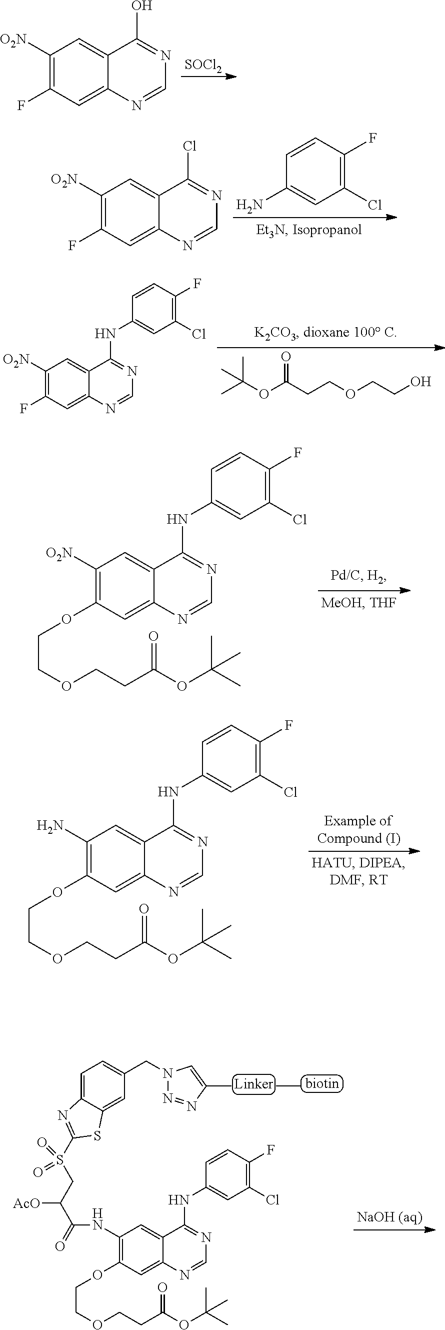

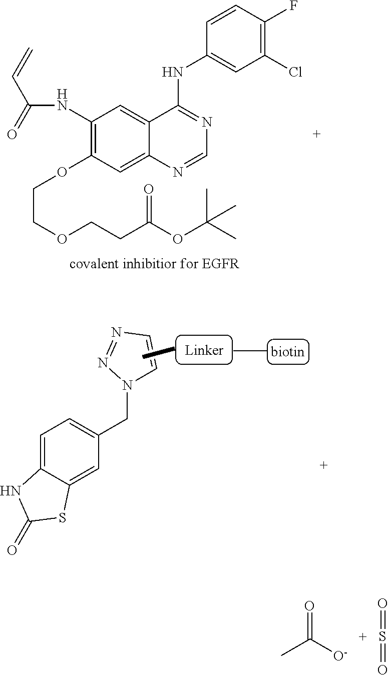

An advanced intermediate of “afatinib” is synthesized as illustrated in the above scheme. The reactions are performed as described in International Patent Publication No. WO 2013/131424.

### Example 43. Reduction to Practice of the Synthesis of a Benzothiazole Sulfone Desthiobiotin-Tagged Masked-Warhead Reagent

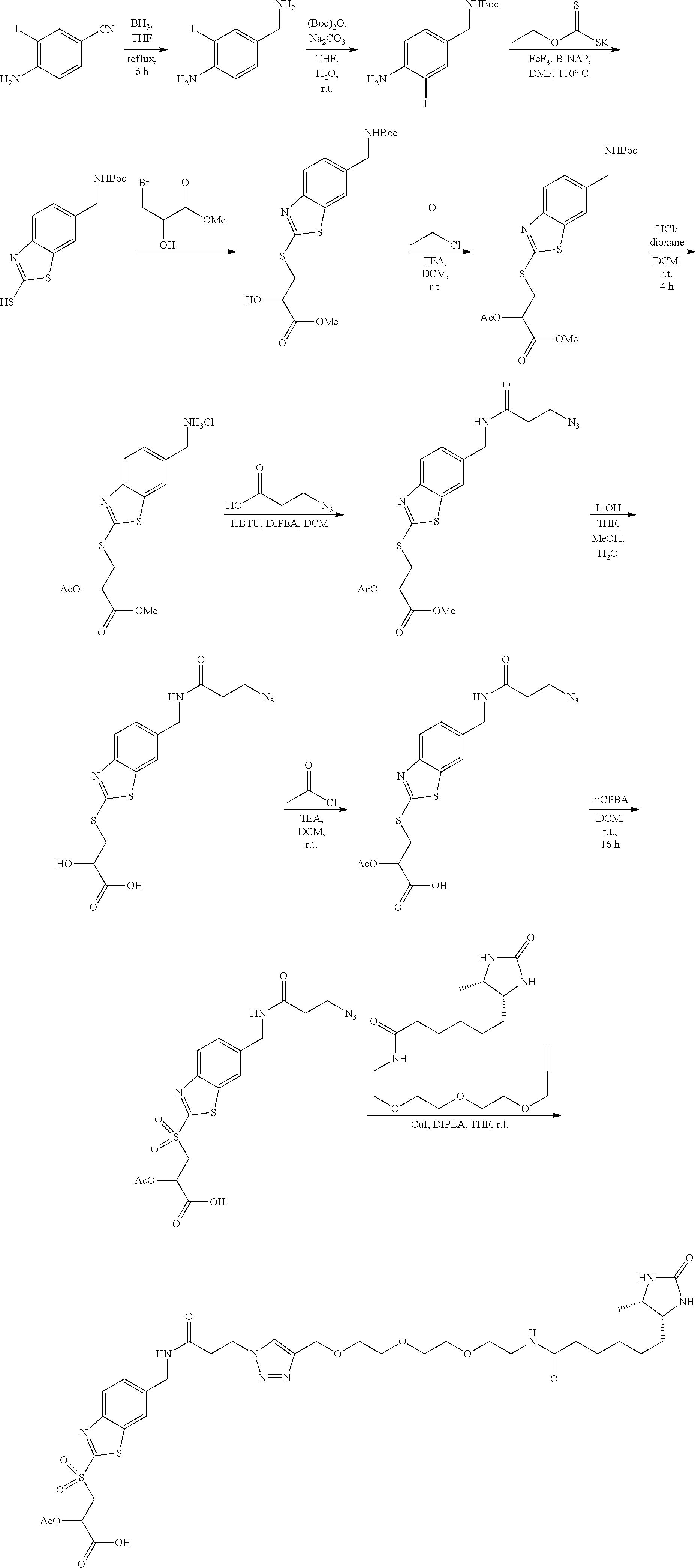

4-(Aminomethyl)-2-iodoaniline: A solution of 4-amino-3-iodobenzonitrile (50.00 g, 205 mmol) in anhydrous THF (200 mL) at room temperature was treated by slow addition of BH₃-THF complex (1M in THF, 410 mL, 410 mmol). The resulting mixture was heated to reflux for 6 hours, then 2 N HCl (200 mL) was added. The mixture was heated to reflux for an additional 1 hour, then cooled to room temperature and extracted with ethyl acetate (100 mL) twice. The combined organic layers were washed twice with brine (80 mL), dried over anhydrous sodium sulfate, filtered and concentrated under reduced pressure to give 4-(aminomethyl)-2-iodoaniline (51.0 g crude yield) as a brown oil, which was directly used for next step without further purification. LCMS analysis: Calculated Exact Mass=248.0. Found [M+H—NH₂]⁺ (ESI+)=232.0.

Tert-butyl (4-amino-3-iodobenzyl)carbamate: A solution of 4-(aminomethyl)-2-iodoaniline (crude, 51.0 g, 205.0 mmoL) and Na₂CO₃ (85.0 g, 615.0 mmol) in THF (300 mL) and H₂O (100 mL) was treated with di-tert-butyl dicarbonate (67.0 g, 307.5 mmol). The mixture was stirred at room temperature overnight. The solvent was then removed under reduced pressure. The mixture was diluted with water (150 mL) and extracted with ethyl acetate (200 mL) twice. The combined organic layers were washed with twice brine (150 mL), dried over anhydrous sodium sulfate, filtered and concentrated under reduced pressure. The residue was purified by silica chromatography (PE/EA=5:1) to give tert-butyl (4-amino-3-iodobenzyl)carbamate (52.0 g, 72.9% yield) as a yellow oil. LCMS analysis: Calculated Exact Mass=348.0. Found [M+H-56]⁺ (ESI+)=293.2.

Tert-butyl ((2-mercaptobenzo[d]thiazol-6-yl)methyl)carbamate: A mixture of tert-butyl (4-amino-3-iodobenzyl)carbamate (52.0 g, 149.35 mmol), potassium o-ethyldithiocarbonate (72.0 g, 448.05 mmol), FeF₃ (1.7 g, 15.00 mmol) and 2,2′-bis(diphenylphosphino)-1,1′-binaphthyl (9.3 g, 15 mmol) in DMF (300 mL) was stirred at 110° C. for 16 hours under a nitrogen atmosphere. After completion, the reaction mixture was adjusted to pH 6 by 1 N HCl, then extracted twice with ethyl acetate (100 mL). The combined organic layers were washed twice with brine (100 mL), dried over anhydrous sodium sulfate, filtered and concentrated under reduced pressure. The residue was purified by silica chromatography (PE/EA=1:1) to give tert-butyl ((2-mercaptobenzo[d]thiazol-6-yl) methyl)carbamate (31.0 g, 70% yield) as a gray solid. LCMS analysis: Calculated Exact Mass=296.1. Found [M+H]⁺ (ESI+)=297.2

Methyl 3-((6-(((tert-butoxycarbonyl)amino)methyl)benzo[d]thiazol-2-yl)thio)-2-hydroxypropanoate: A solution of tert-butyl ((2-mercaptobenzo[d]thiazol-6-yl)methyl) carbamate (13.2 g, 44.50 mmol), methyl 3-bromo-2-hydroxypropanoate (10.5 g, 57.38 mmol) and potassium carbonate (18.4 g, 133.50 mmol) in DMF (100 mL) was stirred at room temperature for 16 hours. The reaction mixture was diluted with water (200 mL) and extracted twice with ethyl acetate (200 mL). The combined organic layers were washed twice with water (200 mL) and twice with brine (200 mL), dried over anhydrous sodium sulfate, filtered and concentrated under reduced pressure. The residue was purified by flash column chromatography (PE/EA=2:1) to give methyl 3-((6-(((tert-butoxycarbonyl)amino)methyl) benzo[d]thiazol-2-yl)thio)-2-hydroxypropanoate (16.5 g, 93%, yield) as a yellow oil. LCMS analysis: Calculated Exact Mass=398.1. Found [M+H]⁺ (ESI+)=399.7.

Methyl 2-acetoxy-3-((6-(((tert-butoxycarbonyl)amino)methyl)benzo[d]thiazol-2-yl)thio)propanoate: A solution of methyl 3-((6-(((tert-butoxycarbonyl)amino)methyl) benzo[d]thiazol-2-yl)thio)-2-hydroxypropanoate (23.0 g, 57.72 mmol) and TEA (11.7 g, 115.44 mmol) in anhydrous DCM (100 mL) cooled at 0° C. was treated by dropwise addition of acetyl chloride (5.4 g, 69.26 mmol). The reaction mixture was stirred at room temperature for 3 hours. The mixture was treated by water (10 mL) and concentrated under reduced pressure. The residue was diluted with water (100 mL) and extracted twice with ethyl acetate (100 mL). The combined organic layers were washed twice with brine (100 mL), dried over anhydrous sodium sulfate, filtered and concentrated under reduced pressure. The residue was purified by flash column chromatography (PE/EA=3:1) to give methyl 2-acetoxy-3-((6-(((tert-butoxycarbonyl)amino)methyl)benzo[d]thiazol-2-yl)thio)propanoate (21.0 g, 82.6% yield) as a brown oil. LCMS analysis: Calculated Exact Mass=440.1. Found [M+H]⁺ (ESI+)=441.1.

Methyl 2-acetoxy-3-((6-(aminomethyl)benzo[d]thiazol-2-yl)thio)propanoate: A solution of methyl 2-acetoxy-3-((6-(((tert-butoxycarbonyl)amino)methyl)benzo[d]thiazol-2-yl)thio)propanoate (21.0 g, 47.67 mmol) in DCM (50 mL) was treated with HCl-dioxane (4 M in dioxane, 50 mL). The reaction mixture was stirred at room temperature for 4 hours. The reaction mixture was then concentrated to give methyl 2-acetoxy-3-((6-(aminomethyl)benzo[d]thiazol-2-yl)thio)propanoate as a white solid (19.8 g, crude, HCl salt), which was directly used for next step without further purification. LCMS analysis: Calculated Exact Mass=340.1. Found [M+H]⁺ (ESI+)=341.1.

Methyl 2-acetoxy-3-((6-((3-azidopropanamido)methyl)benzo[d]thiazol-2-yl)thio) propanoate: A solution of 3-azidopropanoic acid (6.0 g, 52.52 mmol) and O-benzotriazole-N,N,N′,N′-tetramethyl-uronium-hexafluorophosphate (29.9 g, 78.8 mmol) in DCM (100 mL) cooled at 0° C. was treated with N,N-diisopropylethylamine (27.1 g, 2100 mmol). After 30 minutes, the solution was treated with methyl 2-acetoxy-3-((6-(aminomethyl)benzo[d]thiazol-2-yl)thio)propanoate (19.8 g, 52.5 mmol). The reaction solution was allowed to warm to room temperature over 16 hours. The reaction mixture was then diluted with water (100 mL) and extracted twice with DCM (100 mL). The combined organic layers were washed twice with brine (150 mL), dried over anhydrous sodium sulfate, filtered and concentrated under reduced pressure. The residue was purified by flash column chromatography (PE/EA=1:2) to give methyl 2-acetoxy-3-((6-((3-azidopropanamido)methyl)benzo[d]thiazol-2-yl)thio) propanoate (13.0 g, 56.6% yield) as a brown oil. LCMS analysis: Calculated Exact Mass=437.1. Found [M+H]+(ESI+)=438.1.

3-((6-((3-Azidopropanamido)methyl)benzo[d]thiazol-2-yl)thio)-2-hydroxypropanoic acid: A solution of methyl 2-acetoxy-3-((6-((3-azidopropanamido) methyl)benzo[d]thiazol-2-yl)thio)propanoate (13.0 g, 29.74 mmol) in THE (30 mL), methanol (10 mL) and distilled water (10 mL) was treated with lithium hydroxide (2.1 g, 89.22 mmol). The reaction mixture was stirred at room temperature for 2.5 hours. Then the reaction mixture was diluted with ethyl acetate (50 mL), adjusted pH to 5 with 1N HCl and extracted twice with ethyl acetate (100 mL). The combined organic layers were washed with brine (100 mL), dried over anhydrous sodium sulfate, filtered and concentrated under reduced pressure to give 3-((6-((3-azidopropanamido)methyl)benzo[d]thiazol-2-yl)thio)-2-hydroxypropanoic acid (4.3 g, 37.9% yield) as a yellow solid. LCMS analysis: Calculated Exact Mass=381.1. Found [M+H]⁺ (ESI+)=382.0.

2-Acetoxy-3-((6-((3-azidopropanamido)methyl)benzo[d]thiazol-2-yl)thio)propanoic acid: A solution of 3-((6-((3-azidopropanamido)methyl)benzo[d]thiazol-2-yl)thio)-2-hydroxy propanoic acid (4.3 g, 11.27 mmol) and TEA (3.4 g, 33.81 mmol) in anhydrous DCM (30 mL) cooled at 0° C. was treated by dropwise addition of acetyl chloride (1.3 g, 16.91 mmol). The reaction mixture was stirred at room temperature for 3 hours. The mixture was concentrated under reduced pressure. The residue was purified by flash column chromatography (DCM/MeOH=20:1) to give 2-acetoxy-3-((6-((3-azidopropanamido)methyl)benzo[d]thiazol-2-yl)thio)propanoic acid (4.0 g, 83.9% yield) as a yellow oil. LCMS analysis: Calculated Exact Mass=423.1. Found [M+H]⁺ (ESI+)=424.1.

2-Acetoxy-3-((6-((3-azidopropanamido)methyl)benzo[d]thiazol-2-yl)sulfonyl) propanoic acid: A solution of 2-acetoxy-3-((6-((3-azidopropanamido)methyl)benzo[d]thiazol-2-yl)thio)propanoic acid (4.0 g, 9.43 mmol) and 3-chloroperoxybenzoic acid (mCPBA, 4.9 g, 28.29 mmol) in DCM (30 mL) was stirred at room temperature for 16 hours. The reaction was then quenched by saturated aqueous sodium bisulfite (6 mL) and extracted twice with ethyl acetate (50 mL). The combined organic layers were washed twice with brine (30 mL), dried over anhydrous sodium sulfate, filtered and concentrated under reduced pressure. The residue was purified by flash column chromatography (DCM/MeOH=20:1) to give 2-acetoxy-3-((6-((3-azidopropanamido)methyl)benzo[d]thiazol-2-yl)sulfonyl)propanoic acid (2.0 g, 46.5% yield) as a white solid. LCMS analysis: Calculated Exact Mass=455.06. Found [M+H]⁺ (ESI+)=455.95. ¹H NMR (400 MHz, DMSO-d₆) δ 8.68 (t, J=8.0 Hz, 1H), 8.26-8.20 (m, 2H), 7.65-7.62 (m, 1H), 5.44-5.41 (m, 1H), 4.50-4.49 (m, 2H), 4.42-4.35 (m, 1H), 4.29-4.24 (m, 1H), 3.58-3.55 (m, 2H), 2.51-2.48 (m, 2H), 1.64 (s, 3H).

2-Acetoxy-3-((6-((3-azidopropanamido)methyl)benzo[d]thiazol-2-yl)sulfonyl) propanoic acid: A solution of 2-acetoxy-3-((6-((3-azidopropanamido)methyl)benzo[d]thiazol-2-yl)thio)propanoic acid (4.0 g, 9.43 mmol) and 3-chloroperoxybenzoic acid (mCPBA, 4.9 g, 28.29 mmol) in DCM (30 mL) was stirred at room temperature for 16 hours. The reaction was then quenched by saturated aqueous sodium bisulfite (6 mL) and extracted twice with ethyl acetate (50 mL). The combined organic layers were washed twice with brine (30 mL), dried over anhydrous sodium sulfate, filtered and concentrated under reduced pressure. The residue was purified by flash column chromatography (DCM/MeOH=20:1) to give 2-acetoxy-3-((6-((3-azidopropanamido)methyl)benzo[d]thiazol-2-yl)sulfonyl)propanoic acid (2.0 g, 46.5% yield) as a white solid. LCMS analysis: Calculated Exact Mass=455.06. Found [M+H]⁺ (ESI+)=455.95. ¹H NMR (400 MHz, DMSO-d₆) δ 8.68 (t, J=8.0 Hz, 1H), 8.26-8.20 (m, 2H), 7.65-7.62 (m, 1H), 5.44-5.41 (m, 1H), 4.50-4.49 (m, 2H), 4.42-4.35 (m, 1H), 4.29-4.24 (m, 1H), 3.58-3.55 (m, 2H), 2.51-2.48 (m, 2H), 1.64 (s, 3H).

Benzothiazole sulfone desthiobiotin-tagged masked-warhead reagent N-(((1H-1,2,3-triazol-4-yl)methoxy)ethoxy)ethoxy)ethylamido-desthiobiotin)-propanamido)methyl) benzo[d]thiazol-2-yl)sulfonyl)propanoic acid: A solution of 2-acetoxy-3-((6-((3-azidopropanamido)methyl)benzo[d]thiazol-2-yl)sulfonyl)propanoic acid and N-(2-(2-(2-(prop-2-yn-1-yloxy)ethoxy)ethoxy)ethylamido-desthiobiotin (0.5 g, 1.1 mmol) and DIPEA (0.38 mL, 2.2 mmol) in DMF (10 mL) was treated with CuI (20 mg, 0.1 mmol) at room temperature for 16 h. The reaction was diluted with ethyl acetate (50 mL), washed twice with brine (30 mL), dried over anhydrous sodium sulfate, filtered and concentrated under reduced pressure. The residue was purified by preparative HPLC (30% ACN in water) to give N-(((1H-1,2,3-triazol-4-yl)methoxy)ethoxy)ethoxy)ethylamido-desthiobiotin)-propanamido)methyl) benzo[d]thiazol-2-yl)sulfonyl)propanoic acid (94 mg, 95% pure). LCMS analysis: Calculated Exact Mass=838.3. Found [M+H]⁺ (ESI+)=839.3. ¹H NMR (400 MHz, DMSO-d₆) δ 8.69 (t, J=6.4 Hz, 1H), 8.21 (d, J=8.4 Hz, 1H), 8.09 (s, 1H), 8.02 (s, 1H), 7.81 (t, J=6.4 Hz, 1H), 7.54 (dd, J=8.4, 1.6 Hz, 1H), 6.29 (s, 1H), 6.11 (s, 1H), 5.43 (dd, J=8.8, 2.4 Hz, 1H), 4.64-4.61 (m, 2H), 4.51-4.49 (m, 2H), 4.47-4.45 (m, 2H), 4.42-4.35 (m, 1H), 4.28-4.26 (m, 1H), 3.62-3.60 (m, 1H), 3.55-3.51 (m, 4H), 3.50-3.49 (m, 4H), 3.39-3.36 (m, 3H), 3.17 (q, J=6.0 Hz, 2H), 2.86 (t, J=6.8 Hz, 2H), 2.06-2.02 (m, 2H), 1.65 (s, 3H), 1.48-1.42 (m, 2H), 1.35-1.27 (m, 3H), 1.25-1.16 (m, 3H), 0.95 (d, J=6.4 Hz, 3H).

The Examples comprise illustrative single- and multi-step functional group transformations that are well known in the art, as described in, for example, “Comprehensive Organic Synthesis” (B. M. Trost & I. Fleming, eds., 1991-1992) and that may be replaced or superseded by other known transformations and functional groups that nonetheless serve the broader purpose of producing the claimed structures. The modular synthetic routes illustrated in Scheme 1 to Scheme 44 can also be readily modified by one of skill in the art to provide additional substituted masked warheads, acrylamides, and related compounds by conducting functional group transformations on the intermediates and final compounds.

Certain examples in Scheme 1 to Scheme 44 comprise a leaving group (LG) which is a covalently attached group that can be eliminated from the molecule during a chemical reaction (e.g., tosylate, mesylate, acetate, hydroxide, halogens, etc.) (B. M. Trost & I. Fleming, eds., 1991-1992).

The present invention is not limited to any one combinatorial chemistry route or library size chosen to generate DELs, including drug-like small molecule chemical space (Reymond et al, Acc. Chem. Res. 48(3):722-730 (2015)), with the exception that attachment sites should be present on the DEL “scaffolds” or “appendages” to enable acylation by the masked-warhead reagents. Previously, other chemists in the DEL field have also recognized the potential of DELs featuring warheads, and specifically their promise to discover new covalent ligands in unbiased screens for biomedical target proteins (Zimmermann et al., Chem.-Eur. J. 23(34): 8152-8155 (2017); Zambaldo et al., MedChemComm. 7(7):1340-1351 (2016); Kuai et al., SLAS Discov. Adv. Sci. Drug Discov. 23(5):405-416 (2018); Zhu et al., SLAS Discov. Adv. Sci. Drug Discov. 24(2):169-174 (2019); Guilinger et al., Bioorg. Med. Chem. 42:116223 (2021)). Over the past decade DELs have been impactful and useful for reversible ligand discovery (Goodnow et al., Nat. Rev. Drug Discov. 16(2):131-147 (2017)); however, DELs featuring covalent warheads have not been realized for discovery of human therapeutics to-date (Vita, E. D., Future Med. Chem. 13(2), 193-210 (2021); Sutanto et al, RSC Med. Chem. 11(8), 876-884 (2020); Gehringer et al, J. Med. Chem. 62(12):5673-5724 (2020)). Therefore, other chemists in the field have previously attempted brute-forcing warhead acylation reactions on DELs (Guilinger et al., Bioorg. Med. Chem. 42:116223 (2021); Zambaldo et al, MedChemComm. 7(7), 1340-1351 (2016)). Despite selecting DELs known to be particularly favorable this reaction, the outcomes of brute-forcing acylation approaches have been unpurifiable library mixtures, comprising the non-acylated library members as well as synthetic intermediates, capped, truncated, and other byproducts that are nonetheless attached to identical DNA barcodes as the desired warhead products (Guilinger et al., Bioorg. Med. Chem. 42:116223 (2021); Zambaldo et al, MedChemComm. 7(7), 1340-1351 (2016)).

The masked-warhead reagents described herein solve the challenges associated with library-format small-molecule DEL purification by connecting the temporary masking group to a DNA-tag and/or an affinity-tag that are useful to separate the desired warhead-connected eDEL away from non-acylated library members and byproducts, using either gel electrophoresis and/or affinity-based purification methods (Tse et al., J. Am. Chem. Soc. 130:15611-15626 (2008); Usanov et al., Nat. Chem. 10(7):704-714 (2018)). Additionally, the warhead-masking reagents are useful due to their connection to a DNA-tag that enables DNA base-pairing to enforce the acylation reaction under high effective molarity (Gartner et al., Angew. Chem Int. Ed. 42(12):1370-1375 (2003), Gartner et al., Science 305:1601-1605 (2004), Tse et al., J. Am. Chem. Soc. 130:15611-15626 (2008), and Usanov et al., Nat. Chem. 10(7):704-714 (2018)), which will promote the acylation of warheads on a broader scope of low reactivity attachment sites that are typically encountered on diverse small-molecule scaffolds and appendage structures (e.g., anilines, sterically hindered amines, hydroxyls, heterocyclic nitrogens, etc.) (Shi et al., RSC Adv. 11(4):2359-2376 (2021)). The reagents and methods described in the present disclosure will find broad utility in the synthesis of structurally diverse and highly pure DNA-encoded libraries of small molecules featuring electrophilic warheads for covalent ligand discovery.

All patent publications and non-patent publications are indicative of the level of skill of those skilled in the art to which this disclosure pertains. All these publications (including any specific portions thereof that are referenced) are herein incorporated by reference to the same extent as if each individual publication was specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the invention as defined by the appended claims.

What is claimed is:

1. A compound represented by formula (I) or a pharmaceutical salt or stereoisomer thereof: 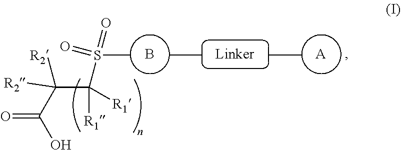
wherein [symbol]
is an oligonucleotide tag represented by [symbol] or an affinity tag; [symbol]
is a linker that covalently attaches [symbol] to [symbol] ; [symbol]
is 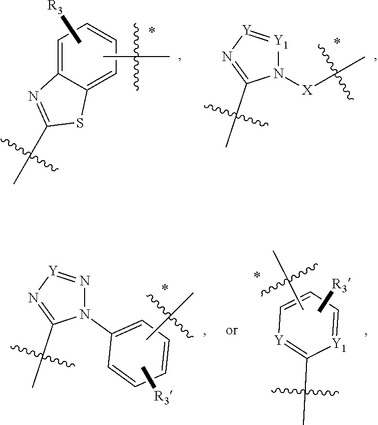
wherein [symbol]
is the connection to the sulfone group, [symbol]
is the connection to the linker,
R₃′ is H, halogen, amino, hydroxyl, (C₁-C₆) alkyl, (C₁-C₆) hydroxyalkyl, (C₁-C₆) aminoalkyl, (C₃-C₆) carbocyclyl, 4- to 6-membered heterocyclyl, (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different Ria groups, wherein each Ria is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl,
X is (C₁-C₆) alkyl, wherein said alkyl is further optionally substituted by one or more, identical or different Ria groups, wherein each Ria is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl, and
Y and Y₁ are each independently CH or N;
R₁′ and R₁″ are each independently H, CH₃, CF₃, halogen, CH₂NMe₂, 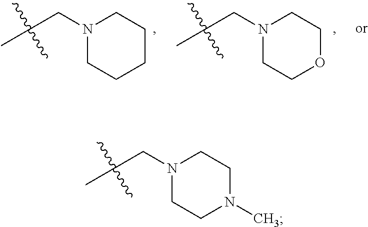 [SMILES: *CN1CCCCC1 ; *CN1CCOCC1 ; *CN1CCN(C)CC1]
R₂′ and R₂″ are each independently H, halogen, CF₃, OH, OAc, CH₂OH, CH(CH₃)OH, C(CH₃)₂OH, CH₂OAc, CH₂OPG, 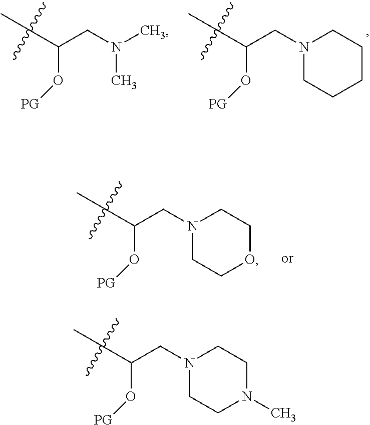
wherein PG is a Protecting Group, provided that both R₂′ and R₂″ are not H, or R₂′ and R₂″ can be joined to form ═O; and
n is 0 or 1.

2. A method of creating an electrophilic warhead-bearing DNA-Encoded Library (eDEL) comprising:
coupling the compound of claim 1 with a DNA-Encoded Library to generate a stable masked-warhead DEL (mwDEL) intermediate;
purifying the mwDEL intermediate;
unmasking the mwDEL intermediate to generate an activated eDEL; and
purifying the activated eDEL.

3. An eDEL, which is generated from the method of claim 2.

4. The eDEL of claim 3, which is used in an in vitro selection assay followed by DNA sequencing.

5. The eDEL of claim 3 or 4, wherein the in vitro selection assay comprises screening protein ligands.

6. The eDEL of claim 3 or 4, wherein the in vitro selection assay comprises screening ligands that covalently modify a residue of a protein.

7. The eDEL of claim 6, wherein the in vitro selection assay comprises screening ligands that covalently modify the thiol group of a Cysteine residue, the imidazole ring of a Histidine residue, the amino group of a Lysine residue, the hydroxyl group of a Serine residue, the hydroxyl group of a Threonine residue, the phenolic hydroxyl of a Tyrosine residue, a carboxylate group, or an amide group of the protein.

8. The eDEL of claim 6, wherein the in vitro selection assay comprises for screening ligands that covalently modify a residue in the active site of the protein.

9. The eDEL of claim 6, wherein the in vitro selection assay comprises screening ligands that covalently modify a residue in a non-orthosteric site of the protein.

10. The eDEL of claim 6, wherein the in vitro selection assay comprises screening ligands that covalently modify a residue in an allosteric site of the protein.

11. The eDEL of claim 6, wherein the in vitro selection assay comprises screening ligands that covalently modify a residue in a non-catalytic domain of a protein.

12. A compound represented by formula (II) or a pharmaceutical salt or stereoisomer thereof: 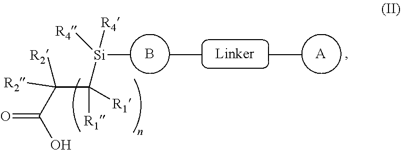
wherein [symbol]
is an oligonucleotide tag represented by [symbol] or an affinity tag; [symbol]
is a linker that covalently attaches [symbol] to [symbol] ; [symbol]
is 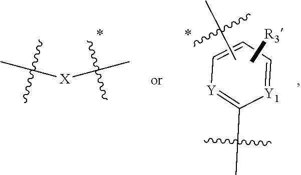
wherein [symbol]
is the connection to the silyl group, [symbol]
is the connection to the linker,
R₃′ is H, halogen, amino, hydroxyl, (C₁-C₆) alkyl, (C₁-C₆) hydroxyalkyl, (C₁-C₆) aminoalkyl, (C₃-C₆) carbocyclyl, 4- to 6-membered heterocyclyl, (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different Ria groups, wherein each Ria is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl,
X is (C₁-C₆) alkyl, wherein said alkyl is further optionally substituted by one or more, identical or different Ria groups, wherein each Ria is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl, and
Y and Y₁ are each independently CH or N;
R₁′ and R₁″ are each independently H, CH₃, CF₃, halogen, CH₂NMe₂, 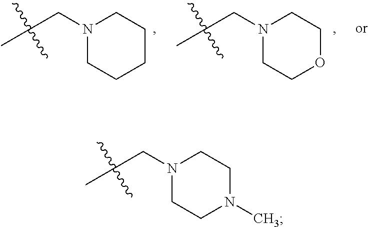 [SMILES: *CN1CCCCC1 ; *CN1CCOCC1 ; *CN1CCN(C)CC1]
R₂′ and R₂″ are each independently H, halogen, CF₃, OH, OAc, CH₂OH, CH(CH₃)OH, C(CH₃)₂OH, CH₂OAc, CH₂OPG, 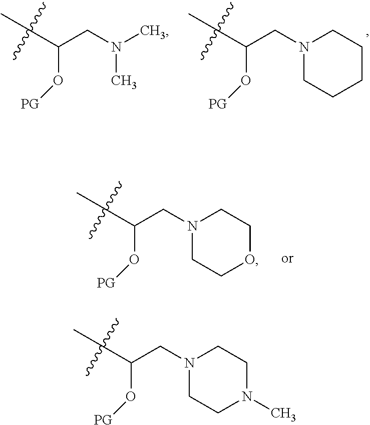
wherein PG is a Protecting Group, provided that both R₂′ and R₂″ are not H, or R₂′ and R₂″ can be joined to form ═O;
R₄′ and R₄″ are each independently alkyl or aryl; and
n is 0 or 1.

13. The compound of claim 12, wherein R₄′ and R₄″ are each independently CH₃, CH₂CH₃, CF₃, propyl, isopropyl, butyl, isobutyl, alkyl, or phenyl.

14. A method of creating an electrophilic warhead-bearing DNA-Encoded Library (eDEL) comprising:
coupling the compound of claim 12 with a DNA-Encoded Library to generate a stable masked-warhead DEL (mwDEL) intermediate;
purifying the mwDEL intermediate;
unmasking the mwDEL intermediate to generate an activated eDEL; and
purifying the activated eDEL.

15. An eDEL, which is generated from the method of claim 14.
